(19) United States
(12) Patent Application Publication
(10) Pub. No.: US 2013/0263294 A1
(43) Pub. Date: Oct. 3, 2013

(54) MARKERS FOR JOINT DISPLASIA, OSTEOARTHRITIS AND CONDITIONS SECONDARY THERETO

(71) Applicants: Antonio Martinez, Derio (ES); Laureano Simon, Derio (ES); Diego Tejedor, Derio (ES); Marta Artieda, Derio (ES); Nerea Bartolome, Derio (ES); Jose Escaich, Barcelona (ES); Alfonso Velasco, Barcelona (ES); Miriam Selles, Barcelona (ES); Carlos Chetrit, Barcelona (ES); Daniel Martinez, Barcelona (ES); Armand Sanchez, Bellaterra (ES); Olga Francino, Bellaterra (ES); Elisenda Sanchez, Bellaterra (ES)

(72) Inventors: Antonio Martinez, Derio (ES); Laureano Simon, Derio (ES); Diego Tejedor, Derio (ES); Marta Artieda, Derio (ES); Nerea Bartolome, Derio (ES); Jose Escaich, Barcelona (ES); Alfonso Velasco, Barcelona (ES); Miriam Selles, Barcelona (ES); Carlos Chetrit, Barcelona (ES); Daniel Martinez, Barcelona (ES); Armand Sanchez, Bellaterra (ES); Olga Francino, Bellaterra (ES); Elisenda Sanchez, Bellaterra (ES)

(73) Assignees: BIOIBERICA, S.A., Barcelona (ES); PROGENIKA BIOPHARMA, S.A., Derio (ES)

(21) Appl. No.: 13/824,842

(22) PCT Filed: Sep. 19, 2011
(86) PCT No.: PCT/EP11/66249

§ 371 Date: Jun. 21, 2013

Related U.S. Application Data
(60) Provisional application No. 61/384,625, filed on Sep. 20, 2010.
(60) Provisional application No. 61/413,239, filed on Nov. 12, 2010.
(60) Provisional application No. 61/497,399, filed on Jun. 15, 2011.

(51) Int. Cl. C12Q 1/68

(52) U.S. Cl. CPC C12Q 1/6881
USPC 800/8; 435/6.11

(57) ABSTRACT

A method for predicting risk of joint dysplasia, osteoarthritis and/or a condition that is secondary to joint dysplasia in a mammalian subject of the order Carnivora, the method comprising: (a) determining the genotype of said subject in respect of one or more genetic polymorphisms and/or alterations, such as polymorphisms in the CHST3 gene; and (b) providing a prediction of said risk based on said genotype. Products for use in such a method and related methods of determining propensity of a subject to respond to therapy and of selective breeding, are also disclosed.

### FIELD OF THE INVENTION

The present invention relates to methods and products, including kits, for determining susceptibility to and/or presence of joint dysplasia, osteoarthritis and/or a condition that is secondary to joint dysplasia. The methods and products of the invention find particular application in relation to mammalian subjects of the order Carnivora, including dogs, and are informative for inter alia personalized treatment, selective breeding and classification of subjects.

### BACKGROUND TO THE INVENTION

The most common form of joint dysplasia in an animal is canine hip dysplasia (CHD), which is a developmental orthopedic disease with an abnormal formation of the hip leads and characterized by varying degrees of hip joint laxity (looseness), subluxation (partial dislocation), and ultimately, severe arthritic change. Hip dysplasia is most common among larger breeds of dogs, especially Labrador Retriever, German Shepherd, Golden Retriever, Beagle, Boxer, Bulldogs, Schnauzers, Rottweiler, Pug, Cocker Spaniel, English Springer Spaniel, Dogues Bordeaux, Bullmastiff, Saint Bernard, Gordon Setter, Bernese mountain dog and American Staffordshire. Until very recently, cats were not thought to be affected by hip dysplasia, but new information and research has shown that this disease does indeed exist in the cat and that, as in dogs, is likely an inherited disorder. Another joint commonly affected by dysplasia, together with the hip, is the elbow. It has been described that there is a moderate and positive genetic correlation between hip and elbow dysplasia (Mäki et al. in J. Anim. Sci. 2000. 78:1141-1148 (2000)). Regardless of the specific joint, hip or elbow, joint dysplasia frequently leads to development of secondary diseases, such as synovitis, muscular atrophy, subcondral bone sclerosis, articular laxitude and osteoarthritis (OA), which causes stiffness, pain and swelling.

Canine hip dysplasia is a complex disease that involves genetic and environmental factors. The diagnosis of CHD is established through radiographic examination of the hip joint. The radiographic methods require a minimum age of the dog at the time of evaluation and detect dysplastic dogs but not dog carriers of the disease. This is why despite in the last decades a high number of dog selection programs based on radiographies have been developed to reduce CHD, there is still a chance of producing a dog with CHD even when their progenitors are free of the disease. A better diagnostic method, such as a genetic test able to detect a dog carrier of the disease is needed.

One of the indexes commonly used for scoring canine hip dysplasia in radiographies is the FCI scoring system which classifies dogs in 5 groups from A, reflecting a normal hip joint, to E, indicating severe hip dysplasia (A: normal hip joint; B: near normal hip joint; C: mild hip dysplasia; D: moderate hip dysplasia and osteoarthritis signs, E: severe hip dysplasia and osteoarthritis signs). The FCI scoring system considers both hip dysplasia and osteoarthritis, since there is a high correlation between severe moderate and severe grades of CHD and the development of osteoarthritis.

The mode of inheritance of canine hip and elbow dysplasia is thought to be polygenic. Mäki et al. in Heredity 92(5):402-8 (2004) described that the inheritance is quantitative, with a major gene affecting the trait jointly with numerous minor genes. Janutta et al. in Journal of Heredity 97(1):13-20 (2006) also found that a mixed model with a dominant major gene in addition to polygenic gene effects seemed to be the most probable for CHD segregation.

Several authors have described quantitative trait loci (QTLs) associated to CHD and/or OA in many chromosomes using microsatellites, single nucleotide polymorphisms (SNPs) or sequence repeat (SSR) as genetic markers (Chase et al. in Am J Med Genet A. 124A(3):239-47 (2004); Chase et al. in Am J Med Gen 135A:334-335 (2005); Mateescu et al. in AJVR, Vol 69: 1294-1300, (2008); Marschall et al. in Mamm Genome. 12:861-70 (2007); Zhu et al. in Anim Genet. 39(2):141-6 (2008)).

Despite the high number of QTLs described as linked to CHD and/or OA, there are few studies assessing the association of specific genes to these diseases. Lee et al. in J. Genet. 86(3):285-8 (2007) analyzed the association of the SLC26A2 gene with CHD and did not find an association. Clements et al. in J Hered. 101(1):54-60 (2010), using SNPs as genetic markers, analyzed the association of several candidate genes, previously described as associated to OA in humans, with canine joint diseases including hip dysplasia. They did not find any significant association for the genes evaluated.

Distl et al. presented in 2008 a patent application (EP2123775A1) related to a process for analysis of the genetic disposition in individuals of the genus Canidae, in relation for hip dysplasia. They describe a list of 17 SNP markers, 2 intergenic and 15 inside a specific gene, associated to CHD and a method for analyzing genetic disposition to CHD based on a sum generated by adding specific numerical values for the 17 markers.

EP2123777A1 relates to a process for analysis of the genetic disposition in individuals of the genus Canidae, in relation for hip dysplasia.

There remains a clear need for methods of predicting susceptibility to CHD and/or OA based on genetic markers. The present invention addresses this need among others.

### BRIEF SUMMARY OF THE INVENTION

The present inventors have now found a strong association between certain genetic polymorphisms and alterations in mammalian subjects of the order Carnivora and the development of joint dysplasia, osteoarthritis and conditions secondary to joint dysplasia. In particular, the risk markers include certain polymorphisms and/or alterations in the CHST3 gene, regulatory regions thereof and in other genes, as described in greater detail herein.

Accordingly, in a first aspect the present invention provides a method of predicting risk of joint dysplasia, osteoarthritis and/or a condition that is secondary to joint dysplasia in a mammalian subject of the order Carnivora, the method comprising:
(a) determining the genotype of said subject in respect of one or more genetic polymorphisms and/or alterations selected from the group consisting of:
   (i) one or more polymorphisms or alterations in the CHST3 gene or a regulatory region thereof;
   (ii) one or more SNPs selected from the SNPs set forth in Tables 2A-C and 12A-D; and
   (iii) one or more polymorphisms or alterations in linkage disequilibrium with (i) or (ii); and
(b) providing a prediction of said risk based on said genotype.

In a second aspect the present invention provides a method of classifying a mammalian subject of the order Carnivora as predisposed or not predisposed to joint dysplasia, osteoarthritis and/or a condition that is secondary to joint dysplasia, the method comprising:
(a) determining the genotype of said subject in respect of one or more genetic polymorphisms and/or alterations selected from the group consisting of:
   (i) one or more (e.g. 2, 3, 4, 5 or more) polymorphisms or alterations in the CHST3 gene or a regulatory region thereof;
   (ii) one or more (e.g. 2, 3, 4, 5, 10, 20 or more) SNPs selected from the SNPs set forth in Tables 2A-C and 12A-D; and
   (iii) one or more polymorphisms or alterations in linkage disequilibrium with (i) or (ii); and
(b) providing a classification of said subject based on said genotype.

By utilising, in particular, specific alterations or risk alleles present in genomic DNA of a subject, the method according to any aspect of the present invention advantageously allows for the identification of, e.g., pre-symptomatic carrier subjects that are predisposed to development of joint dysplasia, OA and/or a condition that is secondary to joint dysplasia. This would not generally be possible with methods that rely on radiographic examination of the hip joint.

The method in accordance with any aspect of the present invention may be carried out in vitro or in vivo. In some cases in accordance with the method of any aspect of the present invention, determining the genotype of said subject comprises assaying a sample that has previously been obtained from said subject. The sample may in general be any suitable biological sample from which the genotype may be determined directly (e.g. by assaying a nucleic acid contained by the sample) or indirectly (e.g. by assaying a protein contained by the sample and from which the genotype of the subject may be inferred). In some cases, the sample is selected from the group consisting of: DNA, urine, saliva, blood, serum, faeces, other biological fluids, hair, cells and tissues.

The genetic variants/variations, alterations or polymorphisms include, but are not limited to, insertion, deletion, repetition and substitution of one or more nucleotides or groups of nucleotides, mutations, including rare mutations (allele frequency <1%) and rearrangements.

In some cases in accordance with the method of any aspect of the present invention, the method comprises determining whether said individual is homozygous or heterozygous for one or more of the risk alleles set forth in Tables 9, 2A-C and 12A-D, or an SNP in linkage disequilibrium with one of said risk alleles.

In some cases in accordance with the method of any aspect of the present invention, the method comprises determining the genotype of said subject in respect of one or more SNPs in the CHST3 gene or a regulatory region thereof, wherein said SNPs are selected from the group consisting of: C38, C18, C34, C32, C36, C17, C15, C6 and C23, as set forth in Table 7, or an SNP in linkage disequilibrium with one of said SNPs.

In some cases in accordance with the method of any aspect of the present invention, the method comprises determining that the subject carries at least one copy of at least one risk allele selected from the group consisting of: G at SNP C38, C at SNP C18 (i.e. presence of G at BICF2P772455 in the TOP strand using Illumina TOP-BOT nomenclature), C at SNP C34, G at SNP C32, G at SNP C36, T at SNP C17, T at SNP C15, T at SNP C6 and T at SNP C23, as set forth in Table 7, or an SNP in linkage disequilibrium with one of said SNP risk alleles.

In some cases in accordance with the method of any aspect of the present invention, determining the genotype of said subject comprises extracting and/or amplifying nucleic acid from a nucleic acid-containing sample that has been obtained from the subject. Generally, but not exclusively, the method may involve extracting and/or amplifying DNA (e.g. genomic DNA or cDNA derived from mRNA).

In some cases in accordance with the method of any aspect of the present invention, determining the genotype of said subject comprises amplifying DNA that has been obtained from the subject by performing PCR using one or more oligonucleotide primers listed in Tables 5 (SEQ ID NOs: 12-23), 6 (SEQ ID NOs: 24-57) and 18 (SEQ ID NOs: 183-199).

In some cases in accordance with the method of any aspect of the present invention, determining the genotype of said subject comprises use of one or more probes as set forth in Table 16 (SEQ ID NOs: 97-182) or Table 17 (SEQ ID NOs: 101, 102, 107, 108, 125, 126, 139, 140, 153, 154, 165, 166, 181, 182). In particular a nucleic acid obtained from the subject or an amplicon derived from a nucleic acid obtained from the subject may be hybridized to one or more of the probes as set forth in Table 16 (SEQ ID NOs: 97-182) or Table 17 (SEQ ID NOs: 101, 102, 107, 108, 125, 126, 139, 140, 153, 154, 165, 166, 181, 182).

In some cases in accordance with the method of any aspect of the present invention, determining the genotype of said subject comprises hybridization, array analysis, bead analysis, primer extension, restriction analysis and/or sequencing.

In some cases in accordance with the method of any aspect of the present invention, determining the genotype of said subject comprises detecting, in a sample that has been obtained from said subject, the presence of a variant polypeptide encoded by a polynucleotide comprising a genetic polymorphism and/or alteration as set forth in Table 14A. The genetic polymorphisms and/or alterations set forth in Table 14A are non-synonymous exonic SNPs which result in at least one amino acid change in the polypeptide product of the respective gene (as set forth in Table 14A). The presence of an amino acid change that corresponds to the respective non-synonymous exonic SNP allows the genotype of the subject to be inferred. In some cases, the presence of the variant polypeptide (e.g. CHST3 polypeptide comprising Arg118Gly) indicates that the subject carries at least one copy of the risk allele G at SNP C32 in the CHST3 gene. Therefore, the presence of said variant polypeptide provides a corresponding indication of risk of or susceptibility to joint dysplasia, OA and/or a condition secondary to joint dysplasia. In some cases, the presence of the variant polypeptide (e.g. CHST3 polypeptide comprising Arg118Gly) indicates that the subject carries at least one copy of mutation, alteration or polymorphism that is different from the risk alleles described herein by virtue of the degeneracy of the genetic code. However, such a mutation, alteration or polymorphism can be expected to also behave as a risk allele for joint dysplasia, OA and/or a condition secondary to joint dysplasia.

In some cases in accordance with the method of any aspect of the present invention, determining the genotype of said subject comprises detecting, in a sample that has been obtained from said subject, the presence of a variant CHST3 polypeptide comprising the amino acid substitution Arg118Gly. In certain cases, presence of the variant CHST3 polypeptide comprising the amino acid substitution Arg118Gly thereby indicates that the genotype of the subject includes the presence of at least one copy of the risk allele G at SNP C32 in the CHST3 gene. In certain cases presence of the variant CHST3 polypeptide comprising the amino acid substitution Arg118Gly thereby indicates that the genotype of the subject includes the presence of at least one copy of a risk allele that is, by virtue of the degeneracy of the genetic code, equivalent to the risk allele G at SNP C32 in the CHST3 gene.

Detecting the presence of the variant polypeptide in accordance with any aspect of the method of the present invention may comprise contacting said sample with an antibody that selectively binds the variant polypeptide.

In some cases in accordance with the method of any aspect of the present invention, determining the genotype of the subject comprises use of a probability function. The use of a probability function may, for example, include a computational method carried out on a combination of outcomes of one or more genetic polymorphisms and/or alterations as defined herein, optionally with one or more clinical outcomes. The computational method may comprise computing and/or applying coefficients or weightings to a combination of said outcomes thereby to provide a probability value or risk indicator. Advantageously, coefficients or weightings for combining the outcomes, e.g. into a predicitive model, may be derived using a “training set” that comprises subjects of known joint status for joint dyplasia, osteoarthritis and/or a condition secondary to joint dysplasia, which once derived may than be applied to a “sample set” that comprises subjects other than the subjects of said training set.

In some cases, the method in accordance with any aspect of the present invention may comprise determining the genotype of said subject in respect of two, three, four, five, six, seven, eight, nine or ten or more genetic polymorphisms and/or alterations as defined herein.

In some cases, the method in accordance with any any aspect of the present invention further comprises obtaining or determining one or more clinical variables that are associated with presence of, or susceptibility to, joint dysplasia, osteoarthritis and/or a condition that is secondary to joint dysplasia. In certain cases, the one or more clinical variables may be selected from the group consisting of: coat colour, adult weight, birth weight, gender, age, exercise habits, diet habits, usual type of floor, early spay, mortality before weaning and litter size.

In certain embodiments, the method in accordance with any aspect of the present invention may comprise determining for said subject the outcome of each of the variables set forth in FIG. 8A, 8B, 8C, 8D, 8E, 8F and/or 8G. The combination of outcomes form predictive models as described further herein. Optionally, the predictive models may themselves be combined.

In a third aspect, the present invention provides a method for determining the propensity of a subject of the order Carnivora to respond effectively to treatment with glycosaminoglycans therapy, the method comprising: determining whether the subject carries at least one copy of at least one risk allele selected from the group consisting of: G at SNP C38, C at SNP C18 (i.e. presence of G at BICF2P772455 in the TOP strand using Illumina TOP-BOT nomenclature), C at SNP C34, G at SNP C32, G at SNP C36, T at SNP C17, T at SNP C15, T at SNP C6 and T at SNP C23, as set forth in Table 7, or an SNP in linkage disequilibrium with one of said SNP risk alleles, wherein the presence of at least one copy of at least one of said risk alleles indicates that said subject has the propensity to respond effectively to said treatment. In accordance with the method of the third aspect of the present invention, the subject may be a subject that has been diagnosed with joint dysplasia (including elbow or hip dysplasia), osteoarthritis and/or a condition secondary to joint dyplasia. However, in certain cases in accordance with the method of the third aspect of the present invention, the subject may not yet have developed or been diagnosed with joint dysplasia (including elbow or hip dysplasia), osteoarthritis and/or a condition secondary to joint dyplasia. In particular, the method of the third aspect of the present invention may be used to identify those subjects that may be suitable for prophylactic treatment with glycosaminoglycans therapy. Such subjects may have been identified as susceptible to with joint dysplasia (including elbow or hip dysplasia), osteoarthritis and/or a condition secondary to joint dyplasia, e.g. using a method in accordance with the first aspect of the invention.

In a fourth aspect, the present invention provides a method of selective breeding comprising:
carrying out the method in accordance with the first or second aspect of the invention on each of a plurality of mammalian subjects of the order Carnivora (e.g. 2, 3, 4, 5, 10, 20, 50, 100 or more mammalian subjects of the order Carnivora), thereby identifying those subjects having increased risk of having or developing joint dysplasia, osteoarthritis and/or a condition that is secondary to joint dysplasia, and those subjects not having said increased risk; and
selectively breeding from those subjects not having said increased risk.

In some cases in accordance with the method of any aspect of the present invention, the subject is Canidae, optionally a dog (Canis familiaris). In certain cases, the subject is a domestic or companion animal such as a dog or cat. The subject may be a pedigree “pure” breed or a mongrel of mixed breed. In certain cases in accordance with the method of any aspect of the present invention, the subject may be greater than 2 kg, greater than 5 kg or greater than 10 kg in weight, or would be expected to be of said weight when fully mature. For example, the subject may be a dog of one or more of the larger breeds. In some cases in accordance with the method of any aspect of the present invention, the subject is a breed of dog selected from the group consisting of: Labrador Retriever, German Shepherd, Golden Retriever, Beagle, Boxer, Bulldogs, Schnauzers, Rottweiler, Pug, Cocker Spaniel, English Springer Spaniel, Dogues Bordeaux, Bullmastiff, Saint Bernard, Gordon Setter, Bernese mountain dog, Saint Bernard and American Staffordshire, or a mongrel breed of dog including one or more of said breeds in its immediate or second or third degree ancestry.

In some cases in accordance with the method of any aspect of the present invention, the subject may have a first or second degree relative (e.g. parent, littermate or offspring) that has joint dysplasia (including elbow or hip dysplasia), osteoarthritis and/or a condition secondary to joint dyplasia.

In some cases in accordance with the method of any aspect of the present invention joint dysplasia is hip and/or elbow dysplasia.

In some cases in accordance with the method of any aspect of the present invention osteoarthritis is primary osteoarthritis, including primary osteoarthritis of the hip and/or elbow.

In some cases in accordance with the method of any aspect of the present invention the condition that is secondary to joint dysplasia is selected from the group consisting of: secondary osteoarthritis, synovitis, muscular atrophy, subcondral bone sclerosis and articular laxitude.

In a fifth aspect the present invention provides an isolated nucleic acid molecule having a polynucleotide sequence that comprises a variant CHST3 gene sequence that has at least 70%, at least 80%, at least 90%, at least 95% or at least 99% sequence identity to the polynucleotide sequence set forth in FIG. 5 (SEQ ID NO: 3), calculated over the full-length of the sequence set forth in FIG. 5 (SEQ ID NO: 3), wherein said variant CHST3 gene sequence comprises at least one substitution corresponding to a substitution selected from the group consisting of: C to T in the SNP C6; G to C in the SNP C34; C to G in the SNP C32; A to G in the SNP C36; and C to T in the SNP C23, wherein said SNPs are as set forth in Table 7. The CHST3 gene may be a canine CHST3 gene, such as a dog CHST3 gene (Canis familiaris).

In a sixth aspect the present invention provides an isolated nucleic acid molecule that is a fragment of the nucleic acid molecule of the fifth aspect, which fragment comprises at least 10, at least 15, at least 20, at least 25, at least 30, at least 35, at least 50, at least 100 or at least 200 contiguous nucleotides of said variant CHST3 gene sequence, wherein said fragment comprises at least one substitution corresponding to a substitution selected from the group consisting of: C to Tin the SNP C6; G to C in the SNP C34; C to G in the SNP C32; A to G in the SNP C36; and C to T in the SNP C23, wherein said SNPs are as set forth in Table 7.

In a seventh aspect the present invention provides a recombinant vector comprising an isolated nucleic acid of the fifth aspect of the invention or an isolated nucleic acid molecule of the sixth aspect of the invention. The vector may comprise said variant CHST3 gene sequence or said fragment thereof, operably linked to a regulatory sequence, e.g. a promoter.

In an eighth aspect the present invention provides a host cell comprising a recombinant vector of the seventh aspect of the invention. In some cases, the host cell may be a mammalian cell. The vector may comprise a nucleic acid sequence that is heterologous to the host cell and/or the vector may be present in a copy number that is altered (e.g. increased or decreased) as compared to the native host cell.

In a ninth aspect, the present invention provides an isolated variant CHST3 polypeptide having at least 70%, at least 80%, at least 90%, at least 95% or at least 99% amino acid sequence identity to the canine CHST3 polypeptide encoded by the CHST3 gene having the polynucleotide sequence set forth in FIG. 5, calculated over the full-length of said canine CHST3 polypeptide, wherein the variant CHST3 polypeptide comprises the amino acid substitution Arg118Gly. The isolated variant CHST3 polypeptide may be a canine polypeptide.

In a tenth aspect, the present invention provides an antibody which selectively binds a variant CHST3 polypeptide of the ninth aspect of the invention. Optionally, the antibody of the tenth aspect displays at least 10-fold binding selectivity (affinity and/or avidity) towards the variant CHST3 polypeptide that comprises the substitution Arg118Gly as compared with the wild-type CHST3 polypeptide encoded by the polynucleotide sequence set forth in FIG. 5. The antibody of the tenth aspect may be a full antibody or a fragment thereof that maintains selective binding to said variant CHST3 polypeptide (e.g. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site; (viii) bispecific single chain Fv dimers (WO 93/11161) and (ix) “diabodies”, multivalent or multispecific fragments constructed by gene fusion (WO94/13804; 58)).

In an eleventh aspect the present invention provides a probe set, comprising a plurality of oligonucleotide probes that interrogate SNPs selected from those set forth in Tables 9, 2A-C and 12A-D, or interrogate an SNP in linkage disequilibrium with one of said SNPs, wherein said oligonucleotide probes make up at least 50% of the oligonucleotide probes in the probe set. In some cases the oligonucleotide probes may be of between 10 and 30 nucleotides in length (e.g. between 15-25 bp). In some cases the probes may span or overlap the polymorphic site or sites. However, it is contemplated herein that the probes may, for example, be directed to or complementary to a contiguous sequence on one side or the other of the polymorphic site. The probe set may comprise pairs of probes wherein one probe of the pair is directed to (e.g. is fully complementary to a first allele of the genetic polymorphism or alteration) a first allele of the genetic polymorphism or alteration while the other probe of the pair is directed to (e.g. is fully complementary to a second allele of the genetic polymorphism or alteration) a second allele of the genetic polymorphism or alteration, i.e. the probes may be “allele-specific” probes.

In certain cases in accordance with this and other aspects of the present invention, the oligonucleotide probes of the probe set may be selected from the probes set forth in Table 16 (SEQ ID NOs: 97-182) or Table 17 (SEQ ID NOs: 101, 102, 107, 108, 125, 126, 139, 140, 153, 154, 165, 166, 181, 182). Advantageously, the probe set comprises one or more probe pairs as set forth in Table 16 (SEQ ID NOs: 97-182) or Table 17 (SEQ ID NOs: 101, 102, 107, 108, 125, 126, 139, 140, 153, 154, 165, 166, 181, 182). The probe pairs set forth in Table 17 (SEQ ID NOs: 101, 102, 107, 108, 125, 126, 139, 140, 153, 154, 165, 166, 181, 182) have been found to exhibit high performance for genotyping their respective SNPs.

In some cases in accordance with the eleventh aspect of the invention the oligonucleotide probes interrogate SNPs selected from the group consisting of: C38, C18, C34, C32, C36, C17, C15, C6 and C23, as set forth in Table 7, or an SNP in linkage disequilibrium with one of said SNPs.

In some cases in accordance with the eleventh aspect of the invention the oligonucleotide probes are provided in the form of an array or are conjugated to a plurality of particles. For example, the probe set may be in the form of a microarray, wherein the probes are deposited on a solid support in an ordered or predetermined pattern. In some cases the probes may be conjugated to beads, such as labelled beads that facilitate detection (e.g. fluorescently labelled beads that are detectable using fluorescence detection).

In some cases in accordance with the eleventh aspect of the invention the probe set is for use in a method according any method of the invention.

In a twelfth aspect, the present invention provides a kit for use in a method of the invention, the kit comprising a plurality of primers selected from those listed in Tables 5, 6 and 18, wherein said primers make up at least 50% of the primers in the kit.

In a thirteenth aspect the present invention provides a genotyping method comprising determining the genotype of one, two, three, four, five or more polymorphisms and/or alterations in the CHST3 gene in a Canidae subject, e.g. a canine subject.

In some cases in accordance with the thirteenth aspect of the invention the one, two, three, four, five or more polymorphisms are SNPs selected from the group consisting of: C38, C18, C34, C32, C36, C17, C15, C6 and C23, as set forth in Table 7, or an SNP in linkage disequilibrium with one of said SNPs.

In some cases in accordance with the thirteenth aspect of the invention the polymorphisms are SNPs selected from the group consisting of: C34, C32, C36, C6 and C23, as set forth in Table 7, or an SNP in linkage disequilibrium with one of said SNPs.

In some cases in accordance with the thirteenth aspect of the invention determining the genotype of said subject comprises extracting and/or amplifying nucleic acid from a nucleic acid-containing sample that has been obtained from the subject.

In some cases in accordance with the thirteenth aspect of the invention determining the genotype of said subject comprises amplifying DNA that has been obtained from the subject by performing PCR using one or more oligonucleotide primers listed in Tables 5 (SEQ ID NOs: 12-23), 6 (SEQ ID NOs: 24-57) and 18 (SEQ ID NOs: 183-199).

In some cases in accordance with the thirteenth aspect of the invention determining the genotype of said subject comprises hybridization, array analysis, bead analysis, primer extension, restriction analysis and/or sequencing.

In some cases in accordance with the thirteenth aspect of the invention the subject is a dog, optionally a dog breed selected from the group consisting of: Labrador Retriever, German Shepherd, Golden Retriever, Beagle, Boxer, Bulldogs, Schnauzers, Rottweiler, Pug, Cocker Spaniel, English Springer Spaniel, Dogues Bordeaux, Bullmastiff, Saint Bernard, Gordon Setter, Bernese mountain dog, Saint Bernard and American Staffordshire, or a mongrel breed of dog including one or more of said breeds in its immediate or second or third degree ancestry.

In yet a further aspect, the present invention provides a probe comprising or consisting of an oligonucleotide sequence set forth in Table 16 (SEQ ID NOs: 97-182) or Table 17 (SEQ ID NOs: 101, 102, 107, 108, 125, 126, 139, 140, 153, 154, 165, 166, 181, 182), or variant thereof. Said variant may comprise or consist of an oligonucleotide sequence that differs from a sequence set forth in Table 16 (SEQ ID NOs: 97-182) or Table 17 (SEQ ID NOs: 101, 102, 107, 108, 125, 126, 139, 140, 153, 154, 165, 166, 181, 182) by 1, 2, 3, 4 or 5 nucleotides by deletion, substitution or insertion.

In yet a further aspect, the present invention provides a primer comprising or consisting of an oligonucleotide sequence set forth in Table 18 (SEQ ID NOs: 183-199), with or without the tag sequence, or variant thereof. Said variant may comprise or consist of an oligonucleotide sequence that differs from a sequence set forth in Table 18 (SEQ ID NOs: 183-199) by 1, 2, 3, 4 or 5 nucleotides by deletion, substitution or insertion.

The present invention includes the combination of the aspects and preferred features described except where such a combination is clearly impermissible or is stated to be expressly avoided.

Section headings are used herein are for convenience only and are not to be construed as limiting in any way.

These and further aspects and embodiments of the invention are described in further detail below and with reference to the accompanying examples and figures.

### DESCRIPTION OF THE FIGURES

FIG. 1 shows the structure of the human (A) and canine (B) CHST3 genes. The position of the SNPs 20 and 21 in the dog genome (B) and in the human genome (A) (position obtained by BLAST alignment tool);

FIG. 2 shows A. Result of the alignment (BLAST) between the human (subject) (SEQ ID NO: 6) and the dog (query) (SEQ ID NO: 7) DNA sequences for the CHST3 gene. The region including the exon 2 of the canine and human CHST3 genes is shown. B. Result of the alignment (BLAST) between the human (subject) (SEQ ID NO: 8) and the dog (query) (SEQ ID NO: 9) DNA sequences for the CHST3 gene. A region including part of the 5′UTR of the human CHST3 gene is shown. The position of the SNP 20 of the dog CHST3 gene (BICF2P772455) is marked by an arrow. C. Result of the alignment (BLAST) between the human (subject) (SEQ ID NO: 10) and the dog (query) (SEQ ID NO: 11) DNA sequences for the CHST3 gene. A region including part of the 3′UTR of the human CHST3 gene is shown. The position of the SNP 21 (BICF2P419109) of the dog CHST3 gene is marked by an arrow;

FIGS. 3A-B shows the location of the primers (described in Table 9) used for the CHST3 gene amplification and sequencing (NCBI: NC_{—}006586.2; Position: 25900637). Exon1 and exon2 are shown by bold letters. Forward primers are highlighted and reverse primers underlined. SEQ ID NOs: 1 & 4;

FIG. 4 shows the sequence of the upstream region and exon1 of the CHST3 gene. A: sequence showing the 640 bp gap of the Boxer Reference sequence (NCBI: NC_{—}006586.2; Position: 25900817) (SEQ ID NO: 5). B: sequence found in the gap in Labrador retrievers. The sequence of the gap is underlined and the exon1 of CHST3 is shown by bold letters. SEQ ID NO 2;

FIGS. 5A-B shows genetic variants found in the CHST3 gene by sequencing of 39 dogs. Genetic variants are highlighted in grey, the sequence corresponding to the gap is underlined and the two exons of the CHST3 gene are shown by bold letters. The variants are numbered and displayed in Table 7 in order of appearance in the sequence. SEQ ID NO 3;

FIG. 6 shows electrophoresis gels showing the PCR band and the RFLP banding pattern for the SNP C32 in individuals with the genotypes CC, CG and GG;

FIG. 7 shows electrophoresis gels showing the PCR band and the RFLP banding pattern for the SNP C38 (BICF2P419109) in individuals with the genotypes CC, CG and GG; and

FIG. 8 shows A. Predictive model (1) for CHD and osteoarthritis. The clinical and genetic variables which remain in the model with the OR (95% IC), the AUC of the ROC, and the sensitivity, specificity, accuracy, and positive and negative predictive values are shown. B. Predictive model (2) for CHD and osteoarthritis. The clinical and genetic variables which remain in the model with the OR (95% IC), the AUC of the ROC, and the sensitivity, specificity, accuracy, and positive and negative predictive values are shown. C. Predictive model (3) for CHD and osteoarthritis. The clinical and genetic variables which remain in the model with the OR (95% IC), the AUC of the ROC, and the sensitivity, specificity, accuracy, and positive and negative predictive values are shown. D. Predictive model (4) for CHD and osteoarthritis. The clinical and genetic variables which remain in the model with the OR (95% IC), the AUC of the ROC, and the sensitivity, specificity, accuracy, and positive and negative predictive values are shown. E. Predictive model (5) for CHD and osteoarthritis. The clinical and genetic variables which remain in the model with the OR (95% IC), the AUC of the ROC, and the sensitivity, specificity, accuracy, and positive and negative predictive values are shown. F. Predictive model (6) for CHD and osteoarthritis. The clinical and genetic variables which remain in the model with the OR (95% IC), the AUC of the ROC, and the sensitivity, specificity, accuracy, and positive and negative predictive values are shown. G. Predictive model (7) for CHD and osteoarthritis. The clinical and genetic variables which remain in the model with the OR (95% IC), the AUC of the ROC, and the sensitivity, specificity, accuracy, and positive and negative predictive values are shown.

### DETAILED DESCRIPTION OF THE INVENTION

In this study, in Labrador retrievers, we have found a strong association of two SNPs near to the 5′ (BICF2P772455) and 3′ (BICF2P419109) ends of the CHST3 gene with CHD and OA. The two SNPs had been previously described as polymorphic in Boxer and Standard poodle, but not in Labrador retrievers (CanFam 2.0 database). BICF2P772455 is located 99 bp upstream of the initial ATG and the SNP096 is located 1051 bp downstream the gene. We have sequenced the dog CHST3 gene and its upstream and downstream regions and found 31 polymorphic SNPs located both in the regulatory regions and inside the gene. Twenty five of the 31 SNPs are believed to be novel SNPs firstly identified in this study and not previously described in the dog genome databases. Together with BICF2P772455 and BICF2P419109, we found that BICF2P772452, BICF2P772454 and 5 of the novel SNPs in the CHST3 gene confer susceptibility to CHD and OA. These SNPs in the CHST3 gene, alone or combined with SNPs in other regions of the genome, allow for determining the risk of a non-human animal, particularly a mammal of the order Carnivora for developing joint dysplasia (such as hip or elbow dysplasia), OA and/or a condition that is secondary to joint dysplasia.

The CHST3 gene, which we found associated to canine HD and OA, has not to our knowledge been previously described as associated with canine hip dysplasia or OA and it is not included inside any of the QTLs found by other authors to be linked to canine HD or OA.

The CHST3 gene encodes a protein involved in chondroitin sulfate (CS) biosynthesis. Chondroitin sulfate is a glycosaminoglycan with a linear polymer structure that possesses repetitive, sulfated disaccharide units containing glucuronic acid (GlcA) and N-acetylgalactosamine (GalNAc). Chondroitin sulfate proteoglycans, such as aggrecan, consist of a core protein with at least 1 covalently attached glycosaminoglycan (GAG) chain and are distributed on the surfaces of most cells and the extracellular matrix in virtually every tissue. The major chondroitin sulfate found in mammalian cartilage has sulfate groups at position C-4 (Chondroitin sulfate A) or C-6 (Chondroitin sulfte C) of the GalNAc residues. CS plays an important role in cartilage function, providing this tissue with resistance and elasticity. Many of their functions are associated with the sulfation profiles of glycosaminoglycans (GAGs). The transfer of sulfate from PAPS (3-prime-phosphoadenosine 5-prime-phosphosulfate) to position 6 of the GalNAc residues rendering Chondroitin sulfate C can be catalyzed by chondroitin 6-sulfotransferase (CHST3 or C6ST) or by chondroitin 6-sulfotransferase 2 (CHST7 or C6ST2), whereas the transfer to position 4 to form chondroitin sulfate A can be mediated by chondroitin 4-sulfotransferase 1 (CHST11 or C4ST1), chondroitin 4-sulfotransferase 2 (CHST12 or C4ST2) or by chondroitin 4-sulfotransferase 3 (CHST13 or C4ST3). It has been shown that during development and ageing and in joint disease occur changes in the structure of CS, affecting the composition of 4- and 6-sulfated disaccharides, (Caterson et al. in J Cell Sci; 97:411-417; 1990). Chondroitin 6-sulfate is related to the integrity of the articular surfaces, whereas chondroitin 4-sulfate is an important factor for calcification process.

Habuchi et al. (EP0745668A2/US5827713) relates to a DNA coding for CHST3/C6ST described as a sulfotransferasa which transfers sulfate groups from a sulfate donor to the hydroxyl group at C-6 position of GalNAc residue or galactose residue of a glycosaminoglycan, preferentially chondroitin. They purified CHST3 from a culture supernatant of chick chondrocytes.

Williams et al. (U.S. Pat. No. 6,399,358B1) describes the DNA encoding human C6ST.

Mutations in the CHST3 gene have been associated in humans with several diseases related to skeletal development, such as spondyloepiphyseal dysplasia (SED Omani type; MIM 608637), recessive Larsen syndrome (MIM 150205) and humerospinal dysostosis (MIM 143095) (Thiele et al. in Proc. Nat. Acad. Sci. vol. 101, 10155-10160, (2004); Hermanns et al. in Am. J. Hum. Genet. vol. 82, 1368-1374, (2008). The mutations described in humans in the CHST3 gene to cause skeletal disorders are not located in the same position as the SNPs in CHST3 gene which we found to be associated to canine hip dysplasia and OA.

In dogs there are no SNPs (CanFam 2.0 and dbSNP-NCBI databases) or genetic variants described inside the CHST3 gene.

The present invention relates to polymorphisms or genetic alterations in the CHST3 and other genes associated to hip and/or joint (hip/joint) dysplasia and osteoarthritis and to a method for determining the risk of an animal for developing hip/joint dysplasia, osteoarthritis and/or a condition that is secondary to joint dysplasia analyzing the genotype of CHST3 and/or other genes alone or in combination with other genetic or clinical variables. The method can be used for predict predisposition or susceptibility to hip/joint dysplasia, osteoarthritis and/or a condition that is secondary to joint dysplasia. The invention provides a method for hip/joint dysplasia and osteoarthritis therapy comprising diagnosing predisposition or susceptibility to hip/joint dysplasia and osteoarthritis, thus allowing differential treatment management for a given individual to prevent or lessen hip/joint dysplasia, osteoarthritis and/or a condition that is secondary to joint dysplasia. The invention can be used to select individuals without or with low predisposition or susceptibility to hip/joint dysplasia, which allows for selecting those individuals for breeding.

In a one aspect the present invention provides a method of diagnosing a disease associated to genetic polymorphisms or variants in the CHST3 (Carbohydrate sulfotransferasa 3) gene in an non-human animal predisposed or susceptible to the disease. Non-limiting examples of a non-human animal are the following ones: dogs, cats, rodents and primates. Preferably, the non-human animal is a mammal of the order Carnivora. An animal predisposed or susceptible to the disease can be an animal which has already developed the disease or a healthy animal which will develop the disease during its life period.

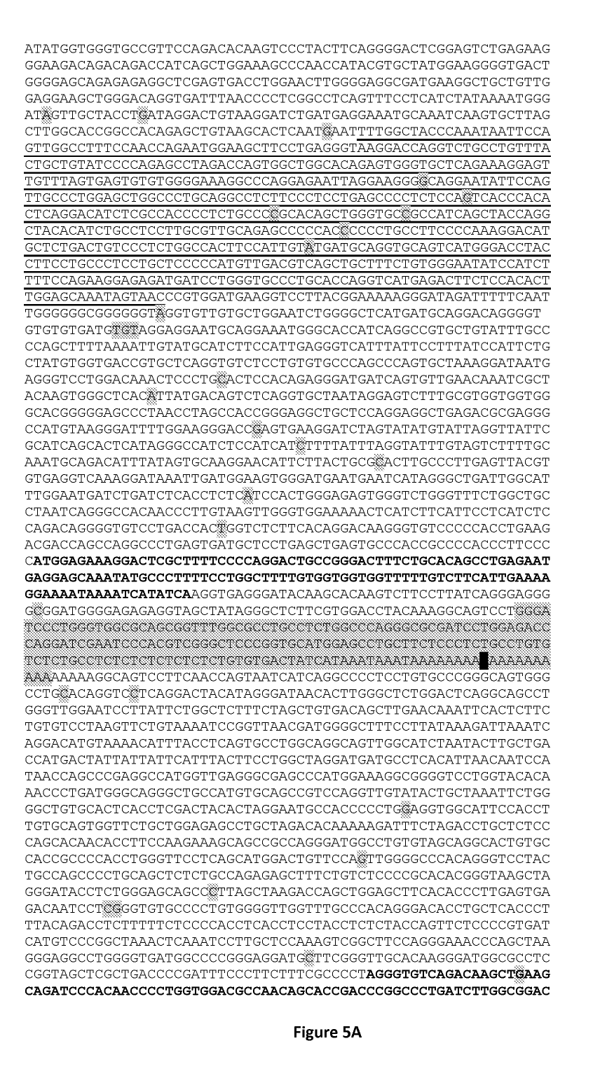

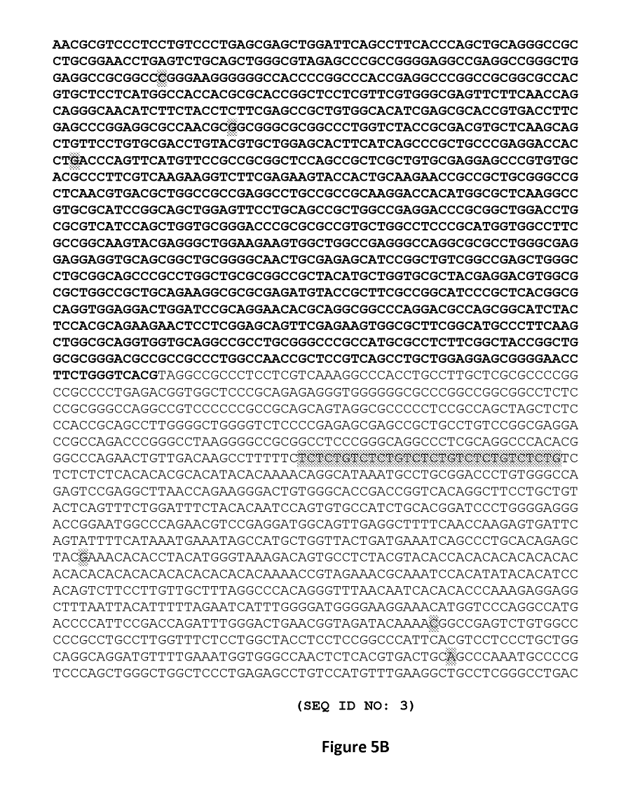

In particular the invention is based upon the observation that one or more single nucleotide polymorphisms (SNPs) within the nucleotide sequence encoding the CHST3 gene, specifically in intron 1, exon 2 and regulatory regions, are correlated to hip dysplasia and osteoarthritis predisposition or susceptibility in individuals of the family Canidae, especially in the genus Canis, i.e. dogs. (see Table 2A, Table 4, Table 7, Table 9 and FIG. 5 (SEQ ID NO: 3)).

The order Carnivora includes placental mammals such as dogs, cats and bears. The family Canidae includes the genus Canis and, in particular, the species Canis familiaris i.e. dogs, such as Labrador retrievers, Golden retrievers, German Sheperd dogs, Beagle, Boxer, Bulldogs, Schnauzers, Rottweiler, Pug, Cocker Spaniel, English Springer Spaniel, Dogues Bordeaux, Bullmastiff, Saint Bernard, Gordon Setter, Bernese mountain dog, Saint Bernard and American Staffordshire, and Canis lupus, i.e. wolfs. Some of the associated SNPs are believed to be new genetic variants described for the first time.

The present invention further provides a method of identifying an animal predisposed or susceptible to hip/joint dysplasia, osteoarthritis and/or a condition that is secondary to joint dysplasia, such as secondary osteoarthritis, said method comprising determining the genotype of the CHST3 gene in said animal.

The term “joint” as used herein refers to a point of articulation between two or more bones, especially such a connection that allows motion, including but not limited to hip, elbow, knee or shoulder.

As used herein a genetic “alteration” may be a variant or polymorphism as described herein.

In some cases the method comprises determining whether an individual is homozygous or heterozygous for SNPs or genetic variants of the CHST3 gene. In an embodiment of the invention, the method is a method of diagnosis for an individual at risk of a condition or disease of hip/joint dysplasia or OA correlated with CHST3 gene polymorphisms or variants. An advantage of this invention is that by screening for the presence of polymorphism is possible to identify at an early stage individuals at risk of developing hip/joint dysplasia, primary osteoarthritis and/or other diseases secondary to hip/joint dysplasia, such as secondary osteoarthritis. The method of invention alone or in combination with others assays, such as radiographic examination, allows for the diagnosis of hip/joint dysplasia and other diseases secondary to hip/joint dysplasia, such as OA at or before disease onset, thus allowing differential treatment management for a given individual to prevent or lessen hip/joint dysplasia and osteoarthritis. The method also provides for prognostic or predictive assays for determining whether an individual is susceptible to develop different grades of hip dysplasia.

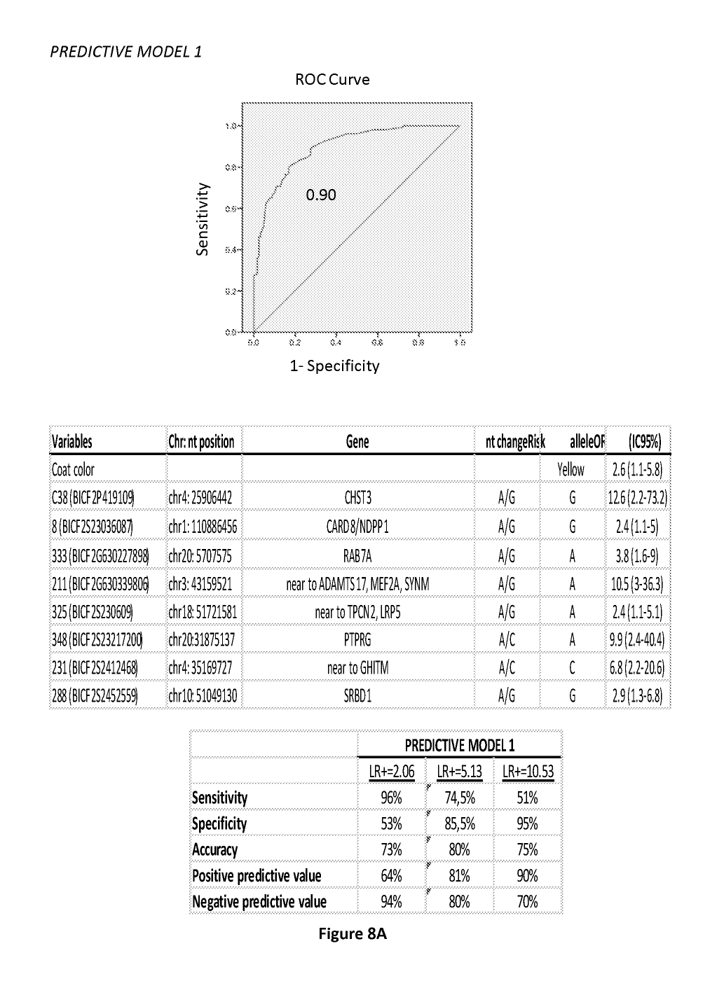

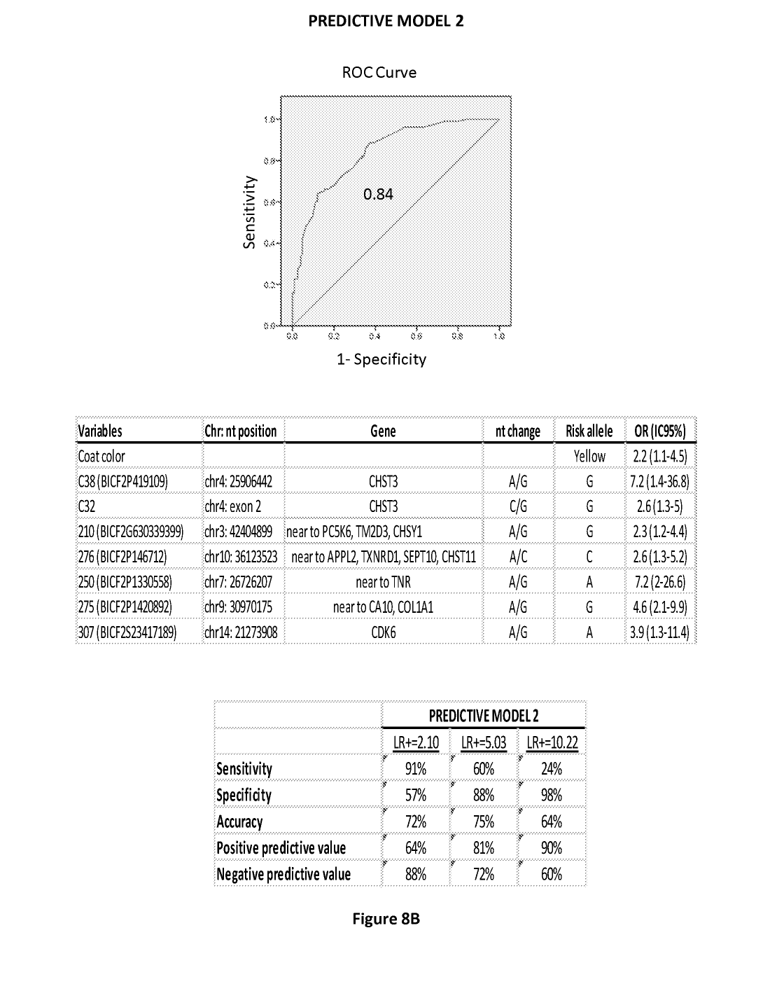

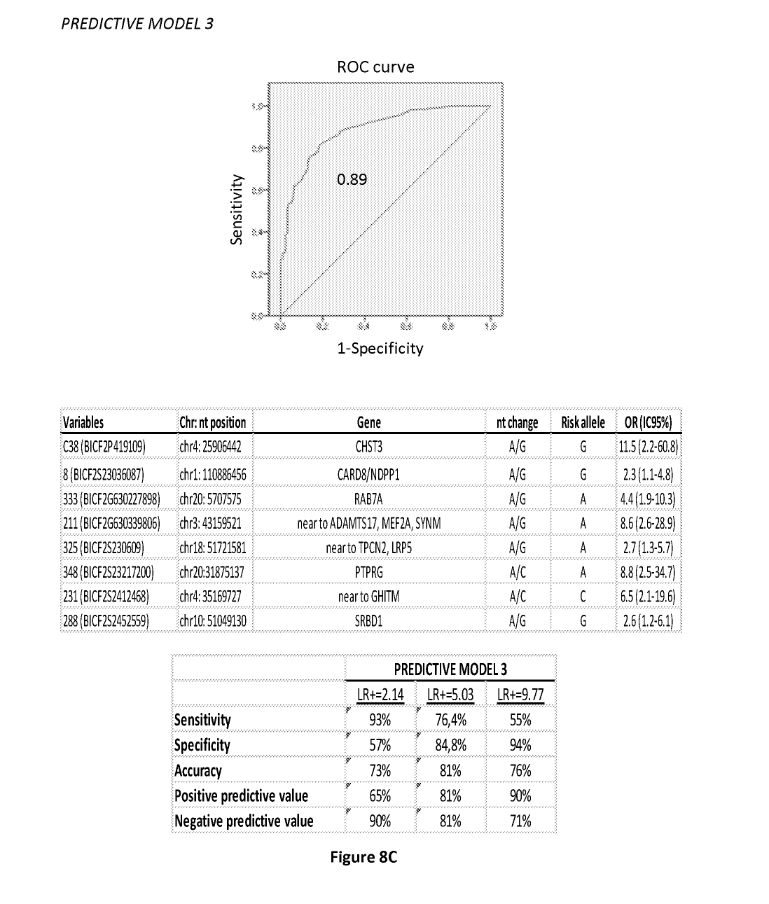

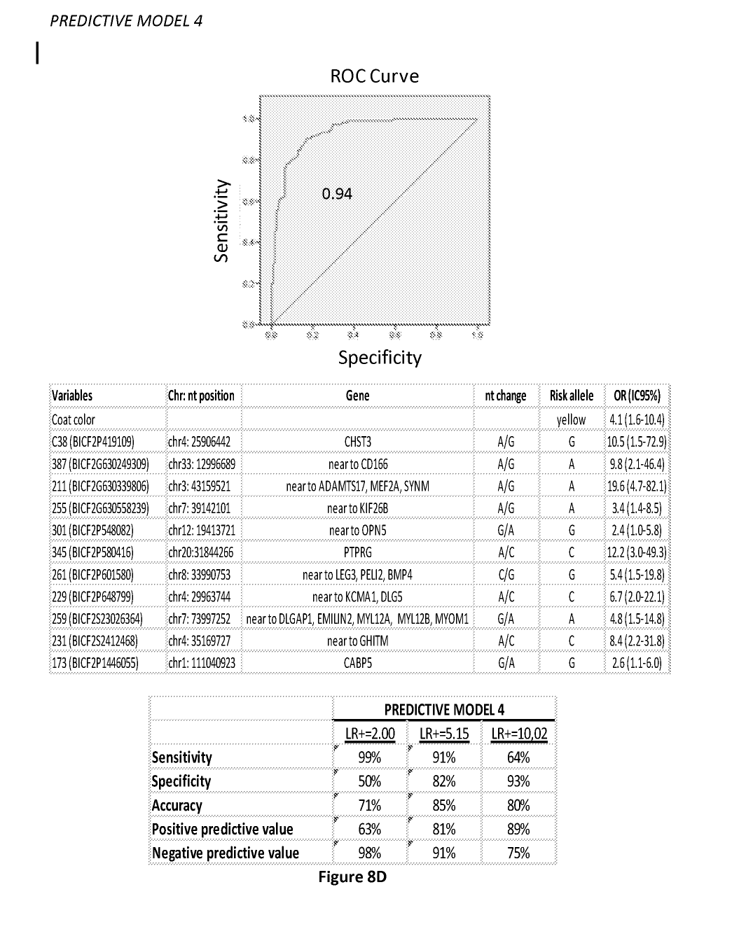

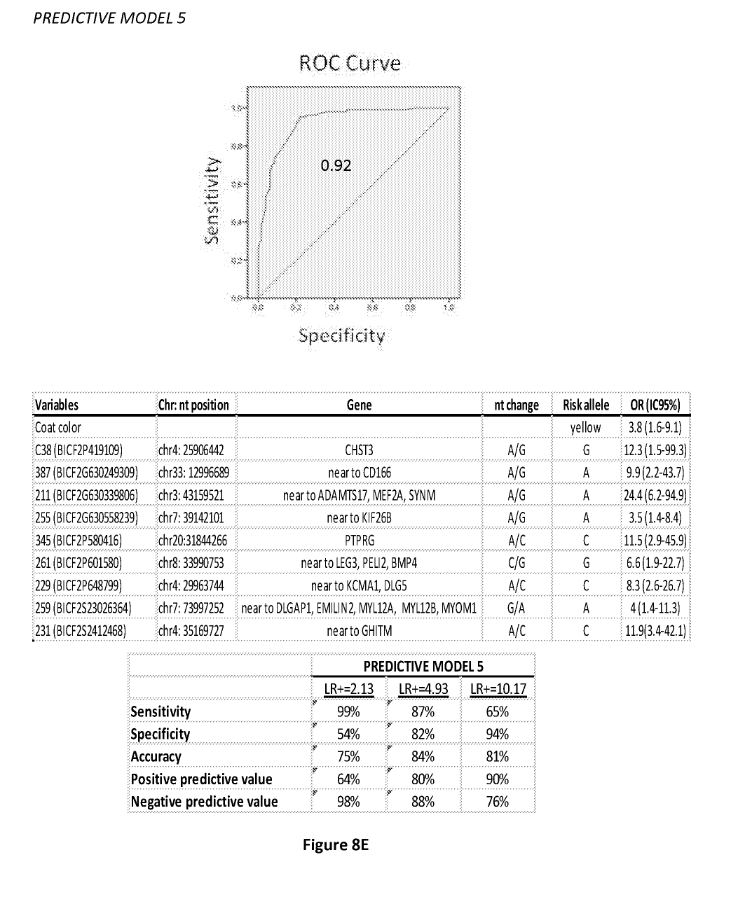

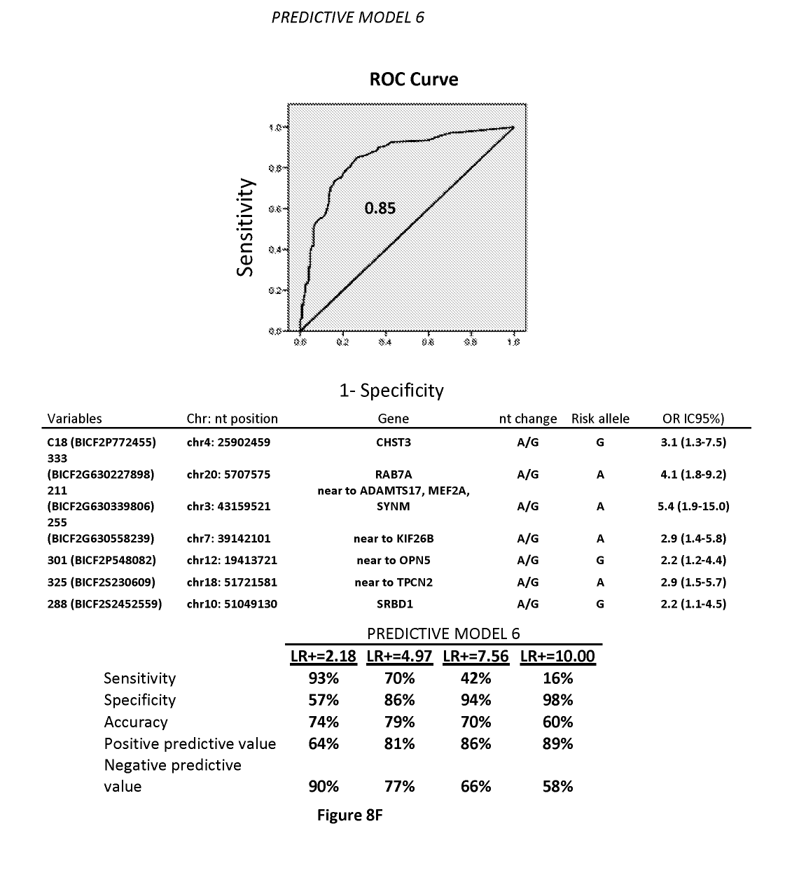

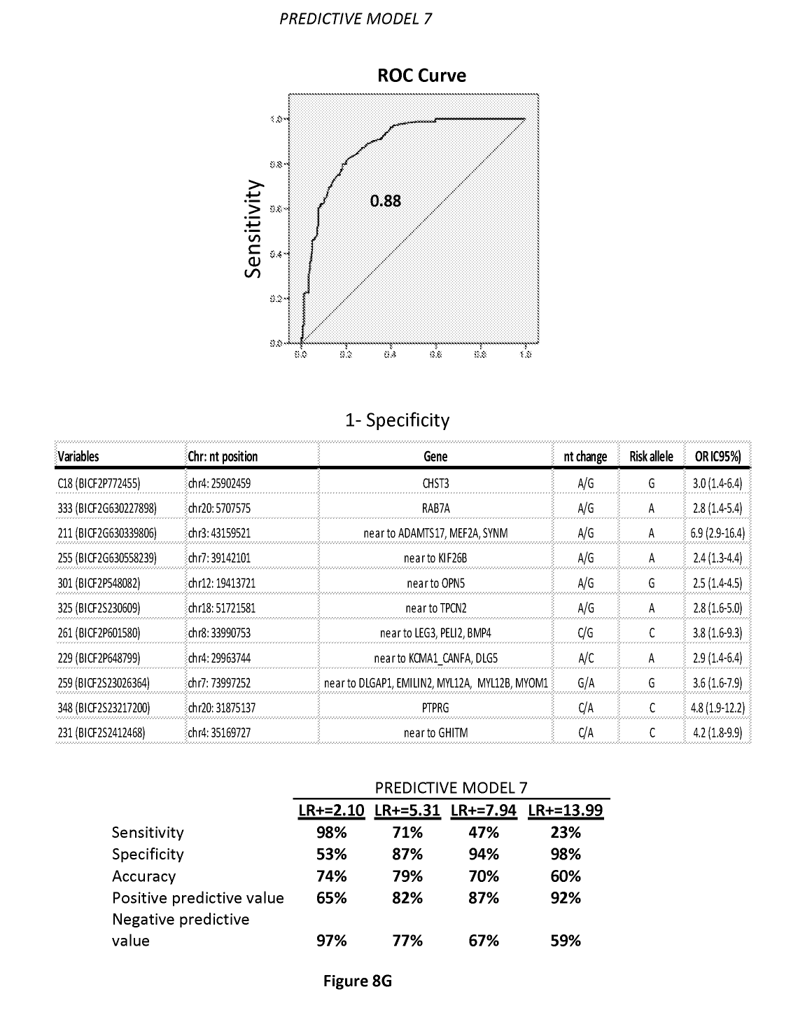

The assessment of an individual's risk factor according to any aspect of the invention can be calculated by determining only the genotype of one or more CHST3 gene polymorphisms or variants and also combining the CHST3 genotype data with analysis of other clinical (e.g. coat colour, adult weight, birth weight, gender, age, exercise habits, diet habits, usual type of floor, early spay, mortality before weaning and litter size) or genetic factors, such as those included in Table 2 A, B, C and D and Table 12 A, B and C. Non-limiting examples of the use of CHST3 genotype alone (Table 2A, Table 4 and Table 9) or in combination with other clinical and genetic factors (FIGS. 8 A, B, C, D and E) are provided.

In an embodiment the invention provides a method that can be used to identify individuals without or with low predisposition or susceptibility to hip/joint dysplasia, which allows for selecting those individuals for breeding.

In another embodiment the invention provides a method for calculating the breeding value, the sum of gene effects of a breeding animal as measured by the performance of its progeny, for a particular individual, based on the genotypes of the invention, to estimate a ranking of the animals as part of a breeding and herd management program.

Accordingly, in an embodiment of the invention the method comprises an isolated nucleic acid molecule containing the total or partial CHST3 nucleic acid sequence (FIG. 5, SEQ ID NO: 3): having one polymorphism as shown in FIG. 5 (SEQ ID NO: 3) and Tables 4, 7 and 9, and SNPs in linkage disequilibrium with them, and its use for hip/joint dysplasia diagnosis or prognosis and other diseases secondary to hip/joint dysplasia, such as osteoarthritis. Thus, the isolated nucleic acid molecule of the invention can have one or a combination of these nucleotide polymorphisms. These nucleotide polymorphisms can also be a part of other polymorphisms in the CHST3 gene that contributes to the presence, absence or severity of hip/joint dysplasia. The isolated nucleic acid molecule of the invention may have a polynucleotide sequence that comprises a variant CHST3 gene sequence that has at least 70%, at least 80%, at least 90%, at least 95% or at least 99% sequence identity to the polynucleotide sequence set forth in FIG. 5 (SEQ ID NO: 3), calculated over the full-length of the sequence set forth in FIG. 5 (SEQ ID NO: 3), wherein said variant CHST3 gene sequence comprises at least one substitution corresponding to a substitution selected from the group consisting of: C to T in the SNP C6; G to C in the SNP C34; C to G in the SNP C32; A to G in the SNP C36; and C to T in the SNP C23, wherein said SNPs are as set forth in Table 7. The CHST3 gene may be a canine CHST3 gene, such as a dog CHST3 gene (Canis familiaris).

The genetic variants/variations, alterations or polymorphisms include, but are not limited to, insertion, deletion, repetition and substitution of one or more nucleotides or groups of nucleotides, mutations, including rare mutations (allele frequency <1%) and rearrangements. If the polymorphism or alteration is in a coding region, it can result in conservative or non-conservative amino acid changes, while if it is in a non-coding region, such as in an intron or in the 3′ and 5′ unstranslated regions can, for example, alter splicing sites, affect mRNA expression or mRNA stability. If the polymorphism in CHST3 results in an amino acid change, the variant polypeptide can be fully functional or can lack total or partial function. The isolated nucleic acid molecules of this invention can be DNA, such as genomic DNA, cDNA, recombinant DNA contained in a vector, or RNA, such as mRNA. The nucleic acid molecule can include all or a portion of the coding sequence of the gene and can further comprise non-coding sequences such as introns and non-conding 3′ and 5′ sequences (including 3′ and 5′ unstranslated regions, regulatory elements and other flanking sequences). The present invention also relates to isolated CHST3 polypeptides, such as proteins, and variants thereof, including polypeptides encoded by nucleotide sequences with the genetic variants described herein (FIG. 5, SEQ ID NO: 3).

As will be appreciated by the reader, in some cases one or more polymorphisms or alterations in linkage disequilibrium with a polymorphism or alteration disclosed herein may find use the methods of the present invention. Linkage disequilibrium (LD) is a phenomenon in genetics whereby two or more mutations or polymorphisms are in such close genetic proximity that they are co-inherited. This means that in genotyping, detection of one polymorphism as present infers the presence of the other. Thus, a polymorphism or alteration in such linkage disequilibrium acts as a surrogate marker for a polymorphism or alteration as disclosed herein. Preferably, reference herein to a polymorphism or alteration in linkage disequilibrium with another means that R²>0.8. Certain preferred LD blocks are set forth in Tables 3, 8, 10 and 13. Therefore, a polymorphism or alteration found within an LD block set forth in Table 3, 8, 10 or 13 will find use the methods of the present invention.

In an embodiment of the invention the method comprises determining whether the CHST3 gene contains the allele G of the polymorphism BICF2P772455 (SNP 20 in Table 2A and SNP C18 in Table 7). An individual is then classified as having an increased risk of predisposition or susceptibility to hip/joint dysplasia and other diseases secondary to hip/joint dysplasia, such as osteoarthritis. Thus, if an individual contains the allele A of the polymorphism BICF2P772455 (SNP 20 in Table 2A and SNP C18 in Table 7) is classified as having decreased risk for hip dysplasia predisposition or susceptibility. Since an individual contains two alleles for the gene CHST3, an individual can be heterozygous or homozygous for the risk allele G.

In another embodiment the invention includes analyzing whether an individual carries in the gene CHST3 the allele G of the polymorphism BICF2P419109 (SNP 21 in Table 2A and SNP C38 in Table 7), wherein being carrier of the allele G correlates with an increased risk of susceptibility or predisposition to hip/joint dysplasia and other diseases secondary to hip/joint dysplasia, such as osteoarthritis, and carrying the allele A with decreased risk. Accordingly, this embodiment includes analyzing whether the CHST3 gene contains a cohesive cleavage site for restriction enzyme PstI (CTGCA/G). A CHST3 gene with a cleavage site for Pstl at that specific position correlates with decreased risk of predisposition or susceptibility to hip/joint dysplasia and other diseases secondary to hip/joint dysplasia, such as osteoarthritis. The lack of this specific cohesive cleavage site (CTGCG/G) correlates with increased risk of susceptibility or predisposition to hip/joint dysplasia and other diseases secondary to hip/joint dysplasia, such as osteoarthritis.

In a further embodiment the method comprises determining whether the CHST3 gene contains the allele C of the polymorphism C34, Leu214Leu, (Table 7 and 9), wherein being carrier of the allele C correlates with an increased risk of susceptibility or predisposition to hip/joint dysplasia and other diseases secondary to hip/joint dysplasia, such as osteoarthritis, and carrying the allele G with decreased risk.

In a further embodiment the method comprises determining whether the CHST3 gene contains the allele G of the polymorphism C32, Arg118Gly, (Table 7 and 9), wherein being carrier of the allele G correlates with an increased risk of susceptibility or predisposition to hip/joint dysplasia and other diseases secondary to hip/joint dysplasia, such as osteoarthritis, and carrying the allele C with decreased risk. Accordingly, this embodiment includes analyzing whether the CHST3 gene contains a blunt cleavage site for restriction enzyme SmaI (CCC/GGG). A CHST3 gene with a cleavage site for SmaI at that specific position correlates with decreased risk of predisposition or susceptibility to hip/joint dysplasia and other diseases secondary to hip/joint dysplasia, such as osteoarthritis. The lack of this specific blunt cleavage site (CCG/GGG) correlates with increased risk of susceptibility or predisposition to hip/joint dysplasia and other diseases secondary to hip/joint dysplasia, such as osteoarthritis.

In a further embodiment the method comprises determining whether the CHST3 gene contains the allele G of the polymorphism C36 (Table 7 and 9), wherein being carrier of the allele G correlates with an increased risk of susceptibility or predisposition to hip/joint dysplasia and other diseases secondary to hip/joint dysplasia, such as osteoarthritis, and carrying the allele A with decreased risk.

In a further embodiment the method comprises determining whether the CHST3 gene contains the allele T of the polymorphism C15 (Table 7 and 9), wherein being carrier of the allele T correlates with an increased risk of susceptibility or predisposition to hip dysplasia, and carrying the allele C with decreased risk.

In a further embodiment the method comprises determining whether the CHST3 gene contains the allele T of the polymorphism C17 (Table 7 and 9), wherein being carrier of the allele T correlates with an increased risk of susceptibility or predisposition to hip/joint dysplasia and other diseases secondary to hip/joint dysplasia, such as osteoarthritis, and carrying the allele A with decreased risk.

In a further embodiment the method comprises determining whether the CHST3 gene contains the allele T of the polymorphism C23 (Table 7 and 9), wherein being carrier of the allele T correlates with an increased risk of susceptibility or predisposition to hip/joint dysplasia and other diseases secondary to hip/joint dysplasia, such as osteoarthritis, and carrying the allele C with decreased risk.

In a further embodiment the method comprises determining whether the CHST3 gene contains the allele T of the polymorphism C6 (Table 7 and 9), wherein being carrier of the allele T correlates with an increased risk of susceptibility or predisposition to hip/joint dysplasia and other diseases secondary to hip/joint dysplasia, such as osteoarthritis, and carrying the allele C with decreased risk.

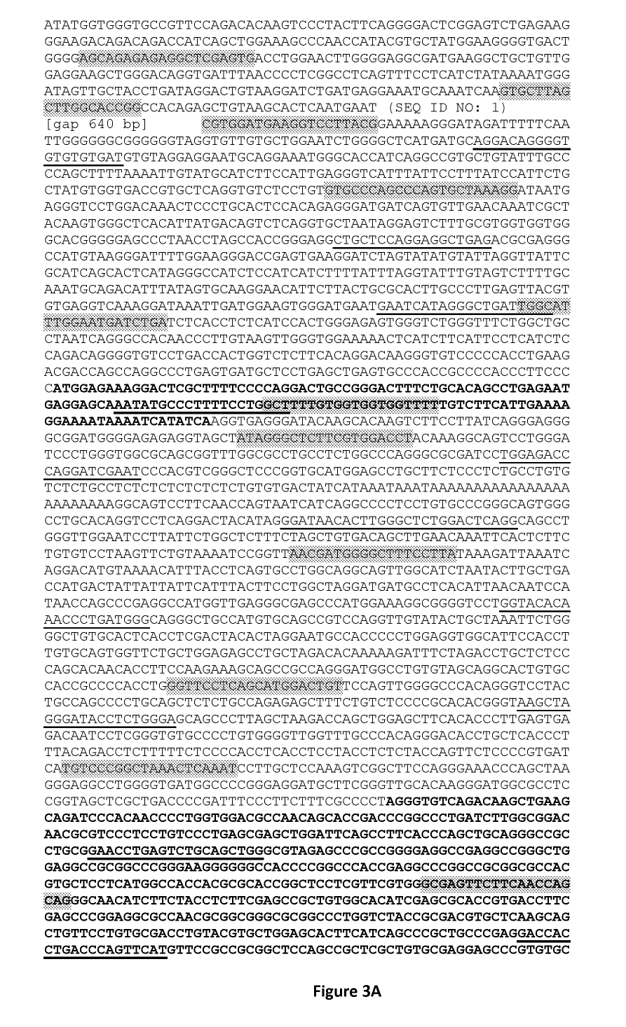

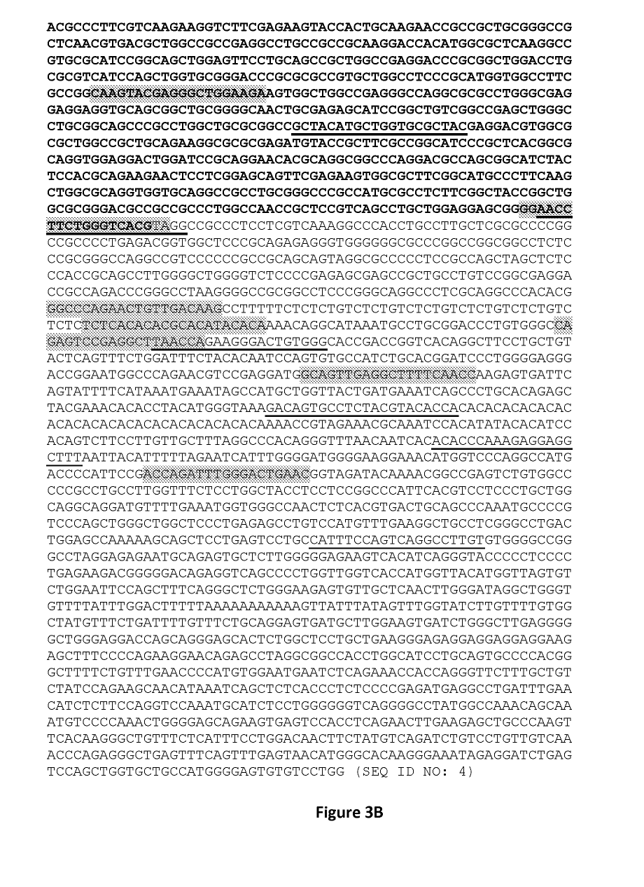

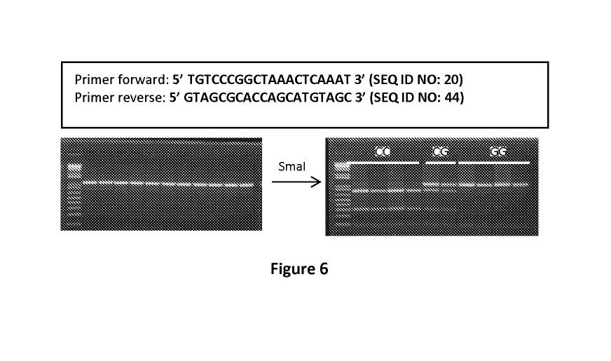

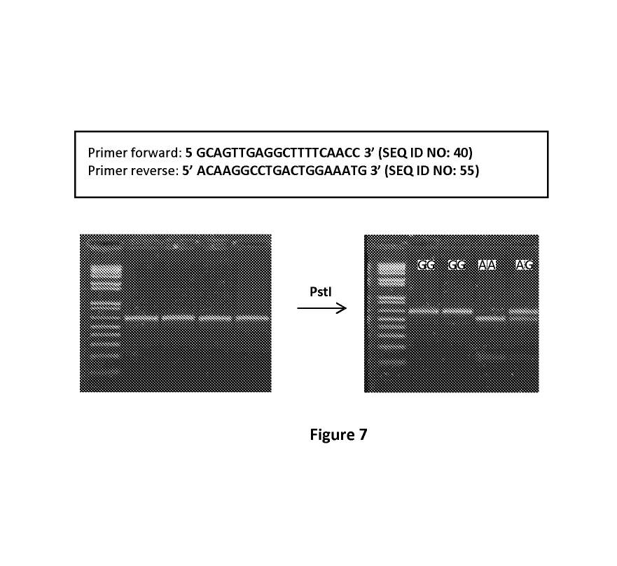

A suitable technique to detect polymorphisms, genetic alterations or variants in the CHST3 gene is analysis by restriction digestion after a PCR reaction for amplifying the region of interest, if the genetic variant or polymorphism results in the creation or elimination of a restriction site (FIGS. 6 and 7). Sequence analysis, such as, direct manual or fluorescent automated sequencing, directly or after selection of the region of interest by PCR, can also be used to detect specific polymorphisms or variants in the CHST3 gene (FIG. 3 (SEQ ID NOs: 1 & 4), 4 (SEQ ID NOs: 2 & 5) and 5 (SEQ ID NO: 3); Tables 6 (SEQ ID NOs: 24-57) and 7). Allele-specific oligonucleotides, for example, used in a competitive PCR, can also be used to detect genetic polymorphisms or variants in CHST3 (Table 9). Another proper technique to detect specific polymorphisms or variants in CHST3 in a sample is testing that sample for the presence of a nucleic acid molecule comprising all or a portion of CHST3 gene, consisting in contacting said sample with a second nucleic acid molecule or probe comprising a nucleotide sequence encoding a CHST3 polypeptide (e.g., FIG. 5 (SEQ ID NO: 3)), a nucleotide sequence encoding a CHST3 polypeptide with comprises at least one polymorphism or genetic variant as shown in FIG. 5 (SEQ ID NO: 3) and Table 7 or genetic polymorphisms and variants in linkage disequilibrium with them, or a fragment, under conditions for selective hybridization. In any of these embodiments, all or a part of the CHST3 gene can be amplified, for example, by PCR, prior to performing the specific technique used for detection of the genetic polymorphisms or variants.

In an embodiment of the invention relates to nucleic acid constructs containing a nucleic acid molecule selected from the SEQ ID NO:1-5 (FIGS. 3-5) and comprising at least one polymorphism as shown in Tables 4 and 7 and FIG. 5 (SEQ ID NO: 3) or polymorphisms in linkage disequilibrium with them, and the complement or a portion thereof. The construct may comprise a vector into which a sequence of the invention has been inserted in sense or antisense orientation.

In an embodiment of the method of the invention includes detecting polymorphisms or variants in the CHST3 gene in a sample from a source selected from the group consisting of: saliva, blood, serum, urine, feces, hair, cells, tissue and other biological fluids or samples.

As indicated above, in some cases in accordance with the method of the invention, the method comprises identifying an animal predisposed or susceptible to hip/joint dysplasia or OA, said method comprising determining the genotype of the CHST3 gene in said animal, and this screening can be performed by a variety of suitable techniques well-known in the art, for example, PCR, sequencing, primer extension, PCR-RFLP, specific hybridization, single strand conformational polymorphism mapping of regions within the gene and PCR using allele-specific nucleotides, among others. In one embodiment oligonucleotide solid-phase based microarray and bead array systems which include probes that are complementary to target nucleic acid sequence can be used to identify polymorphisms or variants in the CHST3 gene. If the polymorphism in CHST3 affects mRNA expression, diagnosis of hip/joint dysplasia and other diseases secondary to hip/joint dysplasia, such as osteoarthritis, can be made by expression analysis using quantitative PCR and Northern blot, among others. If the polymorphism in CHST3 results in an amino acid change, the variant polypeptide can be fully functional or can lack total or partial function. The diagnosis of hip/joint dysplasia and other diseases secondary to hip/joint dysplasia, such as osteoarthritis, can be made by detecting the amino acids essentials for function by methods known in the art, for example, by site-directed mutagenesis or structural analysis, such as nuclear magnetic resonance or antibody-based detection techniques.

A further embodiment of the invention comprises a nucleic acid molecule capable of identifying a polymorphism in said CHST3 gene, said polymorphism being indicative of a risk genotype in said animal. The nucleic acids of the invention are used as probes or primers in assays such as those described herein. Proper primers are, for example, those included in Tables 5 (SEQ ID NOs: 12-23), 6 (SEQ ID NOs: 24-57) and 18 (SEQ ID NOs: 183-199) and FIGS. 3 (SEQ ID NOs: 1 & 4), 6 (SEQ ID NOs: 20 & 44) and 7 (SEQ ID NOs: 40 & 55).

In a still further embodiment, the invention is directed to a diagnostic or prognostic kit for indicating how possessing a polymorphism in CHST3 gene correlates with higher or lower predisposition or susceptibility to hip/joint dysplasia or secondary diseases as osteoarthritis. Kits useful in the methods of diagnosis comprise components useful in any of the methods described herein, such as hybridization probes, restriction enzymes, allele-specific oligonucleotides, antibodies which bind to altered or non-altered CHST3 protein, primers for amplification of nucleic acids, and DNA or RNA polymerase enzymes. Diagnostic assays included herein can be used alone or in combination with other assays, for example, radiographic assays.

In another aspect, the invention provides a method for hip/joint dysplasia and osteoarthritis therapy comprising diagnosing predisposition or susceptibility to hip/joint dysplasia, according to the first aspect of the invention, that is, making an early diagnosis of hip/joint dysplasia at or before disease onset, thus allowing differential treatment management for a given individual to prevent or lessen hip dysplasia and other diseases secondary to hip/joint dysplasia, as osteoarthritis. Nowadays there are several preventive treatment options to prevent or lessen hip/joint dysplasia progression and the appearance of osteoarthritis secondary to hip/joint dysplasia. The preventive therapy options include, among others, weight management by a controlled diet, controlled exercise, massage and physical therapy, anti-inflammatory drugs and chondroprotective drugs, such as glucosamine, hyaluronic acid and glycosaminoglycans, including chondroitinsulfate.

Another aspect of this invention provides a convenient screening system based on CHST3 genetic variants containing the polymorphic site or sites to obtain a substance useful as an agent for treating hip/joint dysplasia or secondary diseases, such as osteoarthritis, and to provide an agent for treating hip/joint dysplasia or secondary diseases containing a substance obtained by the screening system. A non-limiting example is contacting a cultured cell line comprising an allelic variant of the CHST3 gene with an agent capable of treating joint dysplasia and monitoring the expression or processing proteins encoded by the allelic variant of the CHST3 gene.

This invention further relates to therapeutic agents, identified by the above-described screening assays. For example, an agent identified as described herein can be used in an animal model to assess the efficacy, toxicity, mechanisms of action or side effects of treatment with this agent and for treatment of hip/joint dysplasia or secondary diseases, such as osteoarthritis. In one embodiment, an agent useful in a method of the invention can be a polynucleotide. Generally, but not necessarily, the polynucleotide is introduced into the cell, where it effects its function either directly, or following transcription or translation or both. For example, the polynucleotide agent can encode a peptide, which is expressed in the cell and modulates CHST3 activity. A polynucleotide agent useful in a method of the invention also can be, or can encode, an antisense molecule, which can ultimately lead to an increased or decreased expression or activity of CHST3 in a cell, depending on the particular antisense nucleotide sequence. An agent useful for modulating CHST3 expression or activity in a cell can also be a peptide, a peptidomimetic, a small organic molecule, or any other agent.

In another aspect the present invention provides a polynucleotide comprising the reference or variant CHST3 gene sequence, a protein variants encoded by a variant CHST3 polynucleotide, or an antibody against either the reference or variant gene product that contains the polymorphic site or sites, any one or more of which may be incorporated into pharmaceutical composition comprising at least one pharmaceutically acceptable excipient or diluent. The pharmaceutical composition may be suitable for administration in the treatment of hip/joint dysplasia and secondary diseases, such as secondary osteoarthritis. Such compositions can comprise polynucleotides, polypeptides or other therapeutic agents.

In a further aspect, the invention provides a method for determining the propensity of a non-human mammalian subject, optionally of the order Carnivora, to respond effectively to treatment for CHD, primary OA, and/or a disease that is secondary to CHD, such as secondary OA, synovitis, muscular atrophy, subcondral bone sclerosis and articular laxitude, which treatment comprises glycosaminoglycans theraphy, the method comprising determining wether the subject carries at least one risk allele of the SNPs identified in CHST3 as associated to CHD and OA (Tables 4 and 9), wherein the presence of the risk allele indicate a higher propensity to respond effectively to said treatment.

It is possible that the herein presented CHST3 polymorphisms are not the disease causing genetic variants but are instead in linkage disequilibrium with other susceptibility polymorphisms in the CHST3 gene or with a nearby novel disease susceptibility gene on the same chromosome. Nonetheless, the observed association is of use in diagnosis risk of predisposition or susceptibility to hip/joint dysplasia and secondary diseases, such as osteoarthritis.

It is to be understood that the present invention it is not to be limited to the specific forms herein described. It will be apparent to those skilled in the art that various changes may be made without departing from the scope or embodiments of the invention and that the invention is not to be considered limited to what is shown in the drawings and described in the specification.

The invention will be further described by the following non-limiting examples.

### EXAMPLES

### Animals and Phenotype Assessment

The study population consisted of 457 Labrador retrievers, 53 Golden Retrievers and 42 German sheperd dogs. Coat colour, adult weight, birth weight, gender, age, exercise habits, diet habits, usual type of floor, early spay, mortality before weaning and litter size of each dog were registered. Standard ventro-dorsal hip extended radiographies of all dogs were evaluated for CHD and OA by a unique veterinary expert group from the official Spanish Small Animal Veterinary Association (AVEPA) using the FCI scoring system. According to the FCI scoring system, dogs are classified in 5 groups from A, reflecting a normal hip joint, to E, indicating severe hip dysplasia (A: normal hip joint; B: near normal hip joint; C: mild hip dysplasia; D: moderate hip dysplasia and osteoarthritis signs, E: severe hip dysplasia and osteoarthritis signs). Dogs graded as C are mild dysplastic and are the most controversial group, since some experts consider that for association studies they should be classified together with A and B dogs, which are considered non-dysplastic dogs, while others think that they should be included in the dysplastic dogs group, which includes D and E dogs.

### SNP Selection, DNA Isolation and SNP Genotyping

We followed two different strategies to identify the genetic variants associated to CHD: a candidate gene strategy and a genome wide association analysis study (GWAS). To establish the list of candidate genes, we selected genes implicated in the molecular processes involved in CHD (cartilage degradation, inflammation, extracellular matrix metabolism and bone remodeling), in genes known to be associated with osteoarthritis in humans, in genes involved in cartilage and bone diseases in humans and in genes located in quantitative trait loci (QTL) associated with CHD. We selected 2 or 3 SNPs per gene and if there was no SNP described inside the gene we selected SNPs in the flanking regions. We used dbSNP (http://www.ncbi.nlm.nih.gov/projects/SNP) and CanFam2.0 (http://www.broadinstitute.org/science/projects/mammals-models/dog/dog-snps-canfam-20) databases for SNPs selection.

DNA was extracted from blood using the QIAamp DNA Blood Mini Kit from (Qiagen, Hilden, Del.) and quantified with a NanoDrop ND-1000 spectrophotometer (NanoDrop Technologies, Wilmington, Del.). In the candidate gene strategy, 768 SNPs were genotyped using a Illumina Golden Gate Assay (Illumina Inc., San Diego, Calif.) (Fan et al. in Cold Spring Harb Symp Quant Biol. 68:69-78 (2003)).

The genome wide analysis study (GWAS) was performed using the Illumina's Canine HD BeadChip (Illumina Inc., San Diego, Calif.) which includes more than 170,000 SNPs.

### Statistical Analysis

Statistical analyses were performed by using the SPSS v15.0 (SPSS, Chicago, Ill., USA), the PLINK v1.07 (http://pngu.mgh.harvard.edu/purcell/plink/) and the HelixTree (Golden Helix, Bozeman, Mont., USA) softwares. Test for deviation from Hardy-Weinberg equilibrium (HWE) was done for each SNP in the control group of dogs. The chi-squared (χ2) test was used for measuring of pairwise linkage disequilibrium (LD), for performing the association tests between CHD and OA, and allele and genotype frequencies of each SNP and between CHD and OA, and the clinical variables (coat colour, adult weight, birth weight, gender, age, exercise habits, diet habits, usual type of floor, early spay, mortality before weaning and litter size). Odds ratios (OR) were calculated with 95% confidence intervals (CI).

Predictive models were developed by means of forward multivariate logistic regression. CHD and OA grade, as defined by the FCI scoring system, was included as the dependent variable and the most significant baseline clinical and genetic variables were included as independent variables. The goodness-of-fit of the models was evaluated using Hosmer_Lemeshow statistics and their accuracy was assessed by calculating the area under the curve (AUC) of the receiver operating characteristic (ROC) curve. To measure the impact of the SNPs and variables included in the models of the analyzed phenotypes, the sensitivity (S), specificity (Sp) and positive likelihood ratio [LR+=sensitivity/(1_specificity)] were computed by means of the ROC curves.

### Results

In the candidate gene strategy, SNPs with poor genotype cloud clustering or <90% and those which were not in Hardy-Weinberg equilibrium in the population of dogs classified as A (p<0.0001) were excluded. We also excluded samples with an individual genotyping call-rate <90%.

The Labrador retrievers graded as A (n=98) and B (n=134) were over 12 months old at x-ray examination, with a mean age of 34.1 (12-39.9) and 41.5 (23-91.2), for A and B respectively. We did not establish an age limit as inclusion criteria for C, D and E dogs. The mean age of dogs scored as C (n=109), D (n=61) and E (n=47) was 31.5 (6-43.8), 37.2 (6.5-92.2) and 44.9 (6.3-138) months, respectively. Golden retrievers were distributed as follows: A (n=10), B (n=30), C (n=5), D (n=6), E (n=1). German shepherd dogs were distributed as follows: A (n=8), B (n=14), C (n=4), D (n=7), E (n=9).

We performed two distinct allele and genotype association tests in which dogs were classified in two different ways according to their phenotype. First, we carried out the association analysis considering only extreme phenotype dogs (A vs DE), and then we included also the dogs graded as B (AB vs DE).

We found a total of 151 SNPs significantly associated to CHD at the allelic or genotypic level (p<0.05) in at least one of the comparisons (A vs DE or AB vs DE). Specifically, 122 SNPs were associated to CHD when we compared extreme phenotypes, A vs DE, and 114 SNPs when the comparison was made between the AB and the DE Labrador retriever dogs. Most of the SNPs were significantly associated to CHD in both comparisons. The SNPs associated to CHD, their SNP code according to CanFam 2.0 database, the nucleotide change and the chromosomal and gene location are displayed in the Tables 1A, B and C.

We found that some of the SNPs which conferred susceptibility to CHD were in strong linkage disequilibrium (R²>0.8). The SNPs within a same LD block are shown in Table 3. In the Table 1 we have also included those SNPs which, were found to be in LD with the SNPs associated to CHD, rendering a total of 165 SNPs.

Statistical results, p value for χ2 test and OR, of allele and genotype comparisons of the 165 SNPs are given in Tables 2 A, B and C. The risk allele shown in Table 2 corresponds to the TOP strand of the DNA following Ilumina's nomenclature for DNA strand identification. The simplest case of determining strand designations occurs when one of the possible variations of the SNP is an adenine (A), and the remaining variation is either a cytosine (C) or guanine (G). In this instance, the sequence for this SNP is designated TOP. Similar to the rules of reverse complementarity, when one of the possible variations of the SNP is a thymine (T), and the remaining variation is either a C or a G, the sequence for this SNP is designated BOT. If the SNP is an [A/T] or a [C/G], then the above rules do not apply.

Illumina employs a ‘sequence walking’ technique to designate Strand for [A/T] and [C/G] SNPs. For this sequence walking method, the actual SNP is considered to be position ‘n’. The sequences immediately before and after the SNP are ‘n−1’ and ‘n+1’, respectively. Similarly, two base pairs before the SNP is ‘n−2’ and two base pairs after the SNP ‘n+2’, etc. Using this method, sequence walking continues until an unambiguous pairing (A/G, A/C, TIC, or T/G.) is present. To designate strand, when the A or T in the first unambiguous pair is on the 5′ side of the SNP, then the sequence is designated TOP. When the A or T in the first unambiguous pair is on the 3′ side of the SNP, then the sequence is designated BOT.

TABLE 1
SNPs associated to canine hip dysplasia and osteoarthritis. Chromosome position, nucleotide change
and SNP code according to CanFam 2.0 database are shown. The SNP 126 is not included in
CanFam 2.0 database, was selected from dbSNP database.

| SNP number | SNP code (CanFam2.0) | CFA | Gene | Gene region | CFA position (bp) | nt change |
|---|---|---|---|---|---|---|
| 1 | BICF2S23737927 | 1 | ESR1 | Intron | 45367641 | [A/C] |
| 2 | BICF2P930244 | 1 | HAS1 | Intron | 108256911 | [A/G] |
| 3 | BICF2P6947 | 1 | near to SIGLEC12 | 3′ near gene | 108584808 | [A/G] |
| 4 | BICF2P386417 | 1 | SIGLEC12 | Intron | 108592048 | [A/G] |
| 5 | BICF2P104826 | 1 | SIGLEC12 | Intron | 108609585 | [A/G] |
| 6 | BICF2S2302244 | 1 | SNRP70 | Intron | 110231148 | [A/G] |
| 7 | BICF2S2316574 | 1 | SNRP70 | Intron | 110240367 | [A/G] |
| 8 | BICF2S23036087 | 1 | NDPP1-CARD8 | Intron | 110886456 | [A/G] |
| 9 | BICF2S23055347 | 1 | NDPP1-CARD8 | Intron | 110942470 | [A/G] |
| 10 | BICF2S23549799 | 1 | near to QPCTL | 5′ near gene | 112789519 | [A/G] |
| 11 | BICF2P1176847 | 1 | BCAM | Intron | 113467659 | [A/G] |
| 12 | BICF2P1028656 | 1 | PLAUR | Intron | 114379465 | [A/G] |
| 13 | BICF2S23727664 | 1 | TGFB1 | Intron | 115551302 | [A/G] |
| 14 | BICF2P955510 | 1 | near to LTBP4 | 3′ near gene | 116032031 | [A/G] |
| 15 | BICF2P853899 | 3 | CSPG2/VCAN | Exon | 27052514 | [T/A] |
| 16 | BICF2G630339337 | 3 | TM2D3 | Intron | 42281432 | [A/G] |
| 17 | BICF2G630339337 | 3 | TM2D3 | Intron | 42284932 | [A/G] |
| 18 | BICF2G630339349 | 3 | TM2D3 | 3′ UTR | 42292641 | [A/G] |
| 19 | BICF2P525802 | 3 | CSPG1/AGC1 | Exon | 54860309 | [A/G] |
| 20 | BICF2P772455 | 4 | CHST3 | 5′ upstream | 25902459 | [A/G] |
| 21 | BICF2P419109 | 4 | CHST3 | 3′ downstream | 25906442 | [A/G] |
| 22 | BICF2S23042158 | 5 | MIG6/ERRFI1 | Exon | 64623207 | [A/G] |
| 23 | BICF2S2394518 | 5 | MIG6/ERRFI1 | Intron | 64628801 | [G/C] |
| 24 | BICF2P1182878 | 8 | CALM1 | Intron | 64440167 | [A/G] |
| 25 | BICF2P495793 | 9 | ACE | Intron | 14637072 | [A/G] |
| 26 | BICF2P877400 | 9 | c9orf7/LOC491273 | Intron | 53206508 | [A/C] |
| 27 | BICF2P1191819 | 9 | ADAMTSL2 | Intron | 53279761 | [A/C] |
| 28 | BICF2P1469357 | 11 | ADAMTS2 | Intron | 5473064 | [A/G] |
| 29 | BICF2P1267118 | 11 | COL23A1 | Intron | 6150935 | [A/G] |
| 30 | BICF2G630292347 | 11 | LOX | Intron | 15041235 | [A/G] |
| 31 | BICF2S23344397 | 11 | Intergenic | | 16155053 | [A/T] |
| 32 | BICF2G630292963 | 11 | Intergenic | | 16180778 | [A/G] |
| 33 | BICF2P594695 | 11 | CEP120 | Intron | 16195365 | [A/G] |
| 34 | BICF2G630292966 | 11 | CEP120 | Intron | 16255599 | [A/C] |
| 35 | BICF2S23326702 | 11 | Intergenic | | 16356001 | [A/G] |
| 36 | BICF2G630293114 | 11 | CSNK1G3 | Intron | 16364116 | [A/G] |
| 37 | BICF2S23454796 | 11 | CSNK1G3 | Intron | 16404840 | [A/C] |
| 38 | BICF2S23346640 | 11 | Intergenic | | 16486976 | [A/G] |
| 39 | BICF2G630293237 | 11 | Intergenic | | 16497743 | [T/C] |
| 40 | BICF2P1011463 | 11 | Intergenic | | 16583279 | [T/G] |
| 41 | BICF2S23119597 | 11 | Intergenic | | 17054271 | [A/G] |
| 42 | BICF2P509497 | 11 | Intergenic | | 17080356 | [A/G] |
| 43 | BICF2S23334528 | 11 | ZNF608 | Intron | 17335249 | [A/G] |
| 44 | BICF2P334123 | 11 | ZNF608 | Intron | 17366477 | [T/A] |
| 45 | BICF2S23312503 | 11 | Intergenic | | 17420700 | [A/T] |
| 46 | BICF2S23316671 | 11 | Intergenic | | 17686234 | [A/G] |
| 47 | BICF2S23316674 | 11 | Intergenic | | 17686545 | [T/G] |
| 48 | BICF2S23320862 | 11 | Intergenic | | 17689884 | [A/G] |
| 49 | BICF2S2334319 | 11 | Intergenic | | 17714087 | [A/G] |
| 50 | BICF2S2334317 | 11 | Intergenic | | 17714217 | [A/G] |
| 51 | BICF2S23343302 | 11 | Intergenic | | 17760961 | [A/G] |
| 52 | BICF2P307390 | 11 | Intergenic | | 18161063 | [A/G] |
| 53 | BICF2S23349402 | 11 | Intergenic | | 18506514 | [C/G] |
| 54 | BICF2S23349403 | 11 | Intergenic | | 18506579 | [A/C] |
| 55 | BICF2S23349404 | 11 | Intergenic | | 18506762 | [A/C] |
| 56 | BICF2S23349405 | 11 | Intergenic | | 18506930 | [A/G] |
| 57 | BICF2S2331424 | 11 | Intergenic | | 18599717 | [T/A] |
| 58 | BICF2S23334126 | 11 | Intergenic | | 18652508 | [A/T] |
| 59 | BICF2P668886 | 11 | ALDH7A1 | Intron | 18837204 | [A/G] |
| 60 | BICF2S23337722 | 11 | RNUXA | Intron | 18896208 | [A/G] |
| 61 | BICF2S23332527 | 11 | RNUXA | Intron | 18898518 | [A/G] |
| 62 | BICF2S23339286 | 11 | RNUXA | Intron | 18901601 | [A/C] |
| 63 | BICF2S23339285 | 11 | RNUXA | Intron | 18901878 | [A/C] |
| 64 | BICF2G630294733 | 11 | Intergenic | | 18970956 | [C/G] |
| 65 | BICF2G630294806 | 11 | MARCH3 | Intron | 19085130 | [A/G] |
| 66 | BICF2S23750737 | 11 | MARCH3 | Intron | 19106034 | [G/C] |
| 67 | BICF2G630294840 | 11 | MARCH3 | Intron | 19116053 | [A/C] |
| 68 | BICF2S2309790 | 11 | Intergenic | | 19125164 | [A/G] |
| 69 | BICF2S23423736 | 11 | Intergenic | | 19161426 | [A/G] |
| 70 | BICF2S23342048 | 11 | Intergenic | | 19181109 | [A/G] |
| 71 | BICF2S23336733 | 11 | Intergenic | | 19184454 | [A/C] |
| 72 | BICF2S23336732 | 11 | Intergenic | | 19184523 | [G/C] |
| 73 | BICF2S23328632 | 11 | Intergenic | | 19186181 | [A/T] |
| 74 | BICF2S23140493 | 11 | Intergenic | | 19196007 | [A/G] |
| 75 | BICF2G630294921 | 11 | Intergenic | | 19210896 | [A/G] |
| 76 | BICF2G630294961 | 11 | Intergenic | | 19285140 | [A/G] |
| 77 | BICF2S23342754 | 11 | MEGF10 | Intron | 19488082 | [A/G] |
| 78 | BICF2G630295186 | 11 | MEGF10 | Exon | 19562481 | [A/G] |
| 79 | BICF2G630295238 | 11 | Intergenic | | 19599785 | [G/C] |
| 80 | BICF2S23338716 | 11 | PRRC1 | Intron | 19667774 | [A/G] |
| 81 | BICF2S2444318 | 11 | Intergenic | | 19734723 | [A/G] |
| 82 | BICF2G630295313 | 11 | Intergenic | | 19782272 | [A/C] |
| 83 | BICF2P1111143 | 11 | Intergenic | | 19824577 | [A/G] |
| 84 | BICF2G630295635 | 11 | SLC12A2 | 5′UTR | 20147385 | [A/G] |
| 85 | BICF2S23222579 | 11 | SLC12A2 | Intron | 20210746 | [A/G] |
| 86 | BICF2S23245612 | 11 | SLC12A2 | 3′UTR | 20258893 | [A/G] |
| 87 | BICF2S23312270 | 11 | Intergenic | | 20274209 | [A/G] |
| 88 | BICF2P966124 | 11 | FBN2 | Exon | 20547947 | [A/G] |
| 89 | BICF2S23346889 | 11 | Intergenic | | 20575965 | [A/G] |
| 90 | BICF2S23346890 | 11 | Intergenic | | 20576081 | [A/G] |
| 91 | BICF2S23355245 | 11 | Intergenic | | 21068215 | [A/G] |
| 92 | BICF2G630296180 | 11 | ADAMTS19 | Intron | 21400996 | [G/C] |
| 93 | BICF2P258295 | 11 | ADAMTS19/LOC609347 | | 21490420 | [A/G] |
| 94 | BICF2S23310392 | 11 | Intergenic | | 21527441 | [A/C] |
| 95 | BICF2P1435534 | 11 | Intergenic | | 21579805 | [A/C] |
| 96 | BICF2P1425082 | 11 | CHSY3 | Intron | 21627993 | [A/G] |
| 97 | BICF2G630613407 | 13 | HAS2 | 3′UTR | 23348686 | [A/C] |
| 98 | BICF2P1227976 | 13 | HAS2 | 5′UTR | 23376537 | [A/G] |
| 99 | BICF2P968235 | 14 | COL1A2 | Exon | 22844149 | [A/G] |
| 100 | BICF2P998919 | 15 | IGF1 | Intron | 44262267 | [A/G] |
| 101 | BICF2S2334027 | 15 | IGF1 | Intron | 44281937 | [C/G] |
| 102 | BICF2G630217408 | 17 | MATN3 | Exon | 18019587 | [A/G] |
| 103 | BICF2G630207688 | 17 | near to IL-1A | 3′near gene | 40077776 | [A/G] |
| 104 | BICF2P805367 | 17 | CTSK | 3′UTR | 63008191 | [A/G] |
| 105 | BICF2P924791 | 17 | CTSK | Intron | 63009377 | [A/G] |
| 106 | BICF2P282947 | 18 | CTSD | Intron | 49043426 | [A/G] |
| 107 | BICF2P1121207 | 18 | near to PELI3 | 5′near gene | 53897433 | [A/G] |
| 108 | BICF2P1276957 | 18 | near to EFEMP2-FBLN4 | 5′ near gene | 54413084 | [A/C] |
| 109 | BICF2P1382375 | 18 | EFEMP2-FBLN4 | Intron | 54419047 | [A/G] |
| 110 | BICF2P1411014 | 18 | EFEMP2-FBLN4 | Intron | 54419568 | [A/G] |
| 111 | BICF2P386424 | 18 | RELA | Intron | 54581127 | [A/G] |
| 112 | BICF2P915253 | 18 | B3GAT3-GLCAT1 | Intron | 57051032 | [A/G] |
| 113 | BICF2S23632685 | 20 | FLNB | Exon | 35474399 | [A/G] |
| 114 | BICF2P56393 | 20 | FLNB | Intron | 35528817 | [A/G] |
| 115 | BICF2G630448417 | 20 | COL7A1 | Intron | 43548024 | [A/C] |
| 116 | BICF2P139033 | 20 | near to CCR5 | 5′ near gene | 45291955 | [A/G] |
| 117 | BICF2P919318 | 20 | CCR5 | 5′UTR | 45295664 | [A/G] |
| 118 | BICF2S23732829 | 20 | near to CCR2 | 5′near gene | 45307735 | [A/G] |
| 119 | BICF2P153878 | 20 | TNA | Intron | 46327339 | [A/G] |
| 120 | BICF2S23437100 | 22 | LRCH1 | Intron | 7637559 | [A/G] |
| 121 | BICF2G630506640 | 24 | BMP2 | Intron | 18200765 | [A/G] |
| 122 | BICF2P1320955 | 24 | BMP2 | Intron | 18202730 | [A/G] |
| 123 | BICF2P1334955 | 24 | BMP2 | Intron | 18206765 | [G/C] |
| 124 | BICF2P532942 | 24 | near to GDF5 | 5′near gene | 27365690 | [A/G] |
| 125 | BICF2P754855 | 24 | GDF5 | 5′UTR | 27368473 | [A/G] |
| 126 | | 25 | ADAM28 | Exon | 36107108 | [A/G] |
| 127 | BICF2G630101422 | 25 | ADAM28 | Intron | 36161562 | [A/G] |
| 128 | BICF2P1216 | 26 | NCOR2 | Exon | 8451115 | [A/G] |
| 129 | BICF2P178723 | 26 | NCOR2 | Exon | 8496318 | [A/C] |
| 130 | BICF2P133720 | 27 | LRP6 | Intron | 37023147 | [A/T] |
| 131 | BICF2S23547641 | 27 | LRP6 | Intron | 37175397 | [G/C] |
| 132 | BICF2S23336321 | 29 | Intergenic | | 20102334 | [A/G] |
| 133 | BICF2P98408 | 29 | Intergenic | | 20135787 | [A/G] |
| 134 | BICF2P1124539 | 29 | VESTIBULE1 | Intron | 20821352 | [A/C] |
| 135 | BICF2P987772 | 29 | VESTIBULE1 | Intron | 20944564 | [T/A] |
| 136 | BICF2P1087012 | 29 | VESTIBULE1 | Intron | 21262313 | [A/G] |
| 137 | BICF2S23313739 | 29 | VESTIBULE1 | Intron | 21299405 | [T/A] |
| 138 | BICF2S23314747 | 29 | VESTIBULE1 | Intron | 21303089 | [A/G] |
| 139 | BICF2S23314744 | 29 | VESTIBULE1 | Intron | 21303197 | [A/G] |
| 140 | BICF2P160609 | 29 | VESTIBULE1 | Intron | 21321056 | [A/G] |
| 141 | BICF2P1253839 | 29 | Intergenic | | 21396913 | [A/G] |
| 142 | BICF2P360411 | 29 | Intergenic | | 21486987 | [A/C] |
| 143 | BICF2P392807 | 29 | Intergenic | | 21550464 | [A/G] |
| 144 | BICF2P337851 | 29 | Intergenic | | 21589143 | [A/G] |
| 145 | BICF2S23341380 | 29 | Intergenic | | 21589508 | [A/C] |
| 146 | BICF2P337848 | 29 | Intergenic | | 21589638 | [A/G] |
| 147 | BICF2P103219 | 29 | SULF1 | Intron | 21712240 | [A/T] |
| 148 | BICF2S233611 | 29 | SULF1 | Intron | 21743597 | [A/G] |
| 149 | BICF2P1371342 | 29 | SULF1 | Intron | 21755948 | [A/G] |
| 150 | BICF2P643437 | 29 | SULF1 | Exon | 21841767 | [A/G] |
| 151 | BICF2P1067438 | 29 | SULF1 | Intron | 21846016 | [A/G] |
| 152 | BICF2S23543016 | 29 | SULF1 | Intron | 21884261 | [C/G] |
| 153 | BICF2P966484 | 29 | SLCO5A1 | Intron | 21937803 | [A/G] |
| 154 | BICF2P572435 | 29 | SLCO5A1 | Intron | 21941534 | [A/G] |
| 155 | BICF2S23343283 | 29 | SLCO5A1 | Intron | 21979882 | [A/G] |
| 156 | BICF2S23343334 | 29 | SLCO5A1 | Intron | 21983461 | [A/G] |
| 157 | BICF2P779112 | 29 | SLCO5A1 | Intron | 21999748 | [A/G] |
| 158 | BICF2G630403760 | 30 | ADAM10 | Exon | 26634317 | [T/A] |
| 159 | BICF2G630403731 | 30 | ADAM10 | Intron | 26651855 | [A/G] |
| 160 | BICF2G630400865 | 30 | CILP | Intron | 32557793 | [A/G] |
| 161 | BICF2P511492 | 31 | ADAMTS5 | Exon | 25273717 | [A/G] |
| 162 | BICF2P1202421 | 36 | near to FRZB | 5′ near gene | 28924262 | [G/C] |
| 163 | BICF2P226288 | 37 | ADAM23 | Intron | 18009292 | [A/G] |
| 164 | BICF2P968072 | 37 | ADAM23 | Intron | 18115043 | [A/G] |
| 165 | BICF2P99312 | 37 | ADAM23 | Intron | 18159958 | [A/G] |

TABLE 2
SNPs associated to canine hip dysplasia and osteoarthritis: risk allele considering Illumina's TOP strand nomenclature, allele and genotype
association tests results.

| | | | Odds ratio (allele) | | Chi-squared p | | | |
|---|---|---|---|---|---|---|---|---|
| | | | A vs DE | AB vs DE | A vs DE | | AB vs DE | |
| SNP number | Gene | Risk allele | OR (95% CI) | OR (95% CI) | Allele (p) | Genotype (p) | Allele (p) | Genotype (p) |
| 1 | ESR1 | C | 1.56 (1.00-2.44) | 1.70 (1.17-2.46) | 0.049 | 0.065 | 0.005 | 0.011 |
| 2 | HAS1 | A | 1.61 (1.05-2.46) | 1.43 (1.01-2.02) | 0.028 | 0.066 | 0.045 | 0.097 |
| 3 | near to SIGLEC12 | G | 1.19 (0.76-1.84) | 1.25 (0.87-1.81) | 0.449 | 0.051 | 0.229 | 0.024 |
| 4 | SIGLEC12 | G | 1.35 (0.87-2.10) | 1.38 (0.96-2.00) | 0.179 | 0.045 | 0.080 | 0.017 |
| 5 | SIGLEC12 | A | 1.71 (1.02-2.86) | 1.53 (1.01-2.30) | 0.040 | 0.021 | 0.041 | 0.030 |
| 6 | SNRP70 | A | 1.80 (1.21-2.67) | 1.64 (1.18-2.28) | 0.003 | 0.018 | 0.003 | 0.026 |
| 7 | SNRP70 | A | 1.89 (1.26-2.85) | 1.71 (1.22-2.39) | 0.002 | 0.011 | 0.002 | 0.016 |
| 8 | NDPP1-CARD8 | G | 2.03 (1.34-3.09) | 1.72 (1.23-2.41) | 0.001 | 0.001 | 0.001 | 0.002 |
| 9 | NDPP1-CARD8 | A | 1.71 (1.11-2.63) | 1.55 (1.09-2.19) | 0.014 | 0.004 | 0.014 | 0.002 |
| 10 | near to QPCTL | G | 1.59 (1.06-2.39) | 1.56 (1.11-2.18) | 0.025 | 0.133 | 0.009 | 0.062 |
| 11 | BCAM | A | 1.91 (1.26-2.89) | 1.64 (1.16-2.32) | 0.002 | 0.006 | 0.005 | 0.020 |
| 12 | PLAUR | G | 1.84 (1.24-2.73) | 1.59 (1.14-2.21) | 0.002 | 0.010 | 0.006 | 0.037 |
| 13 | TGFB1 | A | 1.72 (1.16-2.54) | 1.76 (1.27-2.45) | 0.007 | 0.024 | 0.001 | 0.004 |
| 14 | near to LTBP4 | G | 1.63 (1.03-2.58) | 1.44 (1.00-2.09) | 0.037 | 0.089 | 0.051 | 0.118 |
| 15 | CSPG2/VCAN | A | 1.56 (1.04-2.33) | 1.70 (1.22-2.37) | 0.030 | 0.052 | 0.002 | 0.008 |
| 16 | TM2D3 | A | 2.21 (1.32-3.70) | 1.67 (1.13-2.48) | 0.002 | 0.012 | 0.010 | 0.045 |
| 17 | TM2D3 | A | 2.51 (1.51-4.18) | 1.87 (1.28-2.75) | 2.95E−04 | 0.004 | 0.001 | 0.007 |
| 18 | TM2D3 | G | 2.51 (1.51-4.18) | 1.87 (1.28-2.75) | 2.95E−04 | 0.004 | 0.001 | 0.007 |
| 19 | CSPG1/AGC1 | G | 1.71 (1.15-2.52) | 1.43 (1.03-1.98) | 0.007 | 0.029 | 0.034 | 0.118 |
| 20 | CHST3 | G | 2.37 (1.58-3.55) | 1.94 (1.39-2.69) | 2.67E−05 | 3.68E−04 | 7.26E−05 | 0.001 |
| 21 | CHST3 | G | 12.26 (2.86-52.59) | 4.70 (2.30-9.60) | 2.33E−05 | 1.17E−04 | 3.96E−06 | 1.18E−05 |
| 22 | MIG6/ERRFI1 | G | 2.47 (0.99-6.20) | 2.82 (1.24-6.39) | 0.047 | 0.143 | 0.010 | 0.038 |
| 23 | MIG6/ERRFI1 | G | 2.06 (1.14-3.71) | 2.13 (1.27-3.58) | 0.015 | 0.015 | 0.003 | 0.014 |
| 24 | CALM1 | A | 6.53 (0.80-53.57) | 2.56 (0.85-7.70) | 0.045 | 0.043 | 0.084 | 0.081 |
| 25 | ACE | G | 1.42 (0.93-2.17) | 1.38 (0.96-1.98) | 0.107 | 0.057 | 0.081 | 0.040 |
| 26 | c9orf7/LOC491273 | C | 1.40 (0.93-2.10) | 1.45 (1.03-2.03) | 0.107 | 0.287 | 0.031 | 0.111 |
| 27 | ADAMTSL2 | C | 1.28 (0.80-2.04) | 1.30 (0.89-1.91) | 0.296 | 0.119 | 0.177 | 0.021 |
| 28 | ADAMTS2 | G | 2.04 (1.22-3.39) | 1.34 (0.91-1.98) | 0.006 | 0.015 | 0.133 | 0.084 |
| 29 | COL23A1 | G | 1.24 (0.77-1.98) | 1.58 (1.07-2.34) | 0.373 | 0.644 | 0.020 | 0.070 |
| 30 | LOX | G | 1.48 (0.99-2.20) | 1.46 (1.05-2.04) | 0.055 | 0.102 | 0.026 | 0.062 |
| 31 | Intergenic | A | 1.50 (0.97-2.33) | 1.46 (1.02-2.09) | 0.070 | 0.060 | 0.037 | 0.070 |
| 32 | Intergenic | G | 1.55 (1.04-2.31) | 1.63 (1.17-2.26) | 0.029 | 0.111 | 0.004 | 0.021 |
| 33 | CEP120 | A | 1.89 (1.15-3.10) | 1.66 (1.13-2.46) | 0.011 | 0.030 | 0.010 | 0.024 |
| 34 | CEP120 | C | 1.51 (1.02-2.23) | 1.53 (1.10-2.11) | 0.039 | 0.102 | 0.011 | 0.033 |
| 35 | Intergenic | A | 1.93 (0.96-3.86) | 1.45 (0.86-2.45) | 0.061 | 0.035 | 0.161 | 0.029 |
| 36 | CSNK1G3 | A | 1.39 (0.93-2.08) | 1.33 (0.95-1.86) | 0.103 | 0.255 | 0.094 | 0.251 |
| 37 | CSNK1G3 | A | 1.41 (0.91-2.17) | 1.51 (1.06-2.16) | 0.119 | 0.232 | 0.023 | 0.024 |
| 38 | Intergenic | G | 1.63 (1.04-2.53) | 1.68 (1.17-2.42) | 0.031 | 0.091 | 0.005 | 0.012 |
| 39 | Intergenic | G | 1.43 (0.93-2.21) | 1.63 (1.13-2.34) | 0.107 | 0.225 | 0.009 | 0.017 |
| 40 | Intergenic | A | 1.59 (0.99-2.55) | 1.63 (1.11-2.40) | 0.052 | 0.145 | 0.011 | 0.014 |
| 41 | Intergenic | G | 1.57 (1.04-2.36) | 1.41 (1.01-1.96) | 0.030 | 0.122 | 0.044 | 0.140 |
| 42 | Intergenic | A | 1.73 (1.15-2.59) | 1.44 (1.02-2.02) | 0.008 | 0.038 | 0.039 | 0.116 |
| 43 | ZNF608 | G | 1.42 (0.95-2.11) | 1.32 (0.95-1.85) | 0.085 | 0.054 | 0.097 | 0.045 |
| 44 | ZNF608 | A | 1.76 (1.01-3.06) | 1.41 (0.92-2.17) | 0.043 | 0.148 | 0.113 | 0.309 |
| 45 | Intergenic | A | 1.83 (0.88-3.78) | 1.42 (0.82-2.46) | 0.100 | 0.059 | 0.213 | 0.038 |
| 46 | Intergenic | A | 1.84 (1.16-2.92) | 1.46 (1.01-2.09) | 0.009 | 0.036 | 0.042 | 0.149 |
| 47 | Intergenic | G | 1.94 (1.23-3.09) | 1.48 (1.03-2.12) | 0.004 | 0.019 | 0.035 | 0.121 |
| 48 | Intergenic | G | 2.16 (1.36-3.45) | 1.64 (1.14-2.36) | 0.001 | 0.005 | 0.007 | 0.024 |
| 49 | Intergenic | A | 1.05 (0.71-1.57) | 1.06 (0.75-1.48) | 0.799 | 0.019 | 0.751 | 0.050 |
| 50 | Intergenic | G | 1.26 (0.85-1.86) | 1.16 (0.84-1.60) | 0.246 | 0.042 | 0.375 | 0.111 |
| 51 | Intergenic | G | 1.61 (1.00-2.59) | 1.33 (0.91-1.95) | 0.049 | 0.068 | 0.134 | 0.057 |
| 52 | Intergenic | A | 1.52 (1.02-2.26) | 1.33 (0.96-1.85) | 0.037 | 0.025 | 0.088 | 0.029 |
| 53 | Intergenic | G | 1.50 (0.93-2.41) | 1.54 (1.04-2.26) | 0.093 | 0.044 | 0.029 | 0.047 |
| 54 | Intergenic | A | 1.60 (0.96-2.67) | 1.62 (1.07-2.45) | 0.069 | 0.035 | 0.021 | 0.029 |
| 55 | Intergenic | A | 1.48 (0.92-2.39) | 1.52 (1.03-2.25) | 0.105 | 0.058 | 0.035 | 0.062 |
| 56 | Intergenic | G | 1.89 (1.09-3.28) | 1.71 (1.11-2.64) | 0.022 | 0.031 | 0.014 | 0.023 |
| 57 | Intergenic | T | 1.49 (0.98-2.28) | 1.49 (1.05-2.12) | 0.064 | 0.003 | 0.026 | 0.017 |
| 58 | Intergenic | T | 1.76 (1.04-2.98) | 1.82 (1.19-2.80) | 0.035 | 0.014 | 0.006 | 0.006 |
| 59 | ALDH7A1 | G | 1.41 (0.89-2.21) | 1.21 (0.84-1.74) | 0.139 | 0.038 | 0.302 | 0.090 |
| 60 | RNUXA | A | 2.10 (1.33-3.32) | 2.06 (1.43-2.98) | 0.001 | 0.003 | 8.93E−05 | 4.71E−04 |
| 61 | RNUXA | A | 2.16 (1.35-3.46) | 2.03 (1.40-2.94) | 0.001 | 0.003 | 1.67E−04 | 0.001 |
| 62 | RNUXA | C | 2.04 (1.29-3.21) | 2.03 (1.41-2.93) | 0.002 | 0.002 | 1.19E−04 | 0.001 |
| 63 | RNUXA | A | 1.99 (1.27-3.14) | 1.98 (1.37-2.86) | 0.003 | 0.004 | 2.12E−04 | 0.001 |
| 64 | Intergenic | G | 1.92 (1.06-3.48) | 1.55 (0.98-2.44) | 0.029 | 0.134 | 0.060 | 0.159 |
| 65 | MARCH3 | A | 2.10 (1.10-4.01) | 1.41 (0.88-2.26) | 0.022 | 0.102 | 0.156 | 0.354 |
| 66 | MARCH3 | C | 2.43 (1.26-4.68) | 1.51 (0.95-2.41) | 0.007 | 0.045 | 0.081 | 0.128 |
| 67 | MARCH3 | A | 1.70 (1.14-2.52) | 1.32 (0.94-1.84) | 0.008 | 0.044 | 0.105 | 0.282 |
| 68 | Intergenic | G | 1.64 (1.11-2.43) | 1.26 (0.91-1.74) | 0.012 | 0.048 | 0.168 | 0.238 |
| 69 | Intergenic | G | 2.46 (1.27-4.73) | 1.48 (0.93-2.36) | 0.006 | 0.042 | 0.097 | 0.134 |
| 70 | Intergenic | A | 1.60 (0.98-2.62) | 1.32 (0.89-1.95) | 0.059 | 0.203 | 0.169 | 0.196 |
| 71 | Intergenic | C | 1.69 (1.15-2.50) | 1.32 (0.95-1.83) | 0.008 | 0.037 | 0.093 | 0.251 |
| 72 | Intergenic | C | 1.44 (0.87-2.37) | 1.19 (0.79-1.77) | 0.154 | 0.399 | 0.405 | 0.638 |
| 73 | Intergenic | A | 1.66 (1.01-2.75) | 1.32 (0.89-1.96) | 0.046 | 0.095 | 0.170 | 0.095 |
| 74 | Intergenic | G | 1.89 (1.15-3.10) | 1.42 (0.97-2.08) | 0.011 | 0.026 | 0.072 | 0.059 |
| 75 | Intergenic | G | 1.92 (1.24-2.98) | 1.41 (0.96-2.07) | 0.003 | 0.021 | 0.080 | 0.243 |
| 76 | Intergenic | G | 1.69 (1.10-2.59) | 1.63 (1.15-2.31) | 0.017 | 0.076 | 0.006 | 0.023 |
| 77 | MEGF10 | G | 1.87 (1.26-2.79) | 1.38 (1.00-1.91) | 0.002 | 0.010 | 0.051 | 0.167 |
| 78 | MEGF10 | A | 2.05 (1.22-3.46) | 1.64 (1.10-2.46) | 0.006 | 0.023 | 0.014 | 0.049 |
| 79 | Intergenic | G | 1.66 (0.27-10.06) | 1.17 (0.22-6.06) | 0.576 | 0.573 | 0.855 | 0.855 |
| 80 | PRRC1 | A | 1.60 (1.08-2.37) | 1.47 (1.05-2.04) | 0.019 | 0.057 | 0.023 | 0.088 |
| 81 | Intergenic | G | 1.59 (1.01-2.50) | 1.48 (1.02-2.13) | 0.043 | 0.143 | 0.036 | 0.107 |
| 82 | Intergenic | A | 1.50 (1.01-2.22) | 1.25 (0.89-1.74) | 0.046 | 0.117 | 0.192 | 0.296 |
| 83 | Intergenic | A | 1.53 (1.03-2.28) | 1.30 (0.93-1.82) | 0.036 | 0.074 | 0.129 | 0.268 |
| 84 | SLC12A2 | A | 1.65 (1.01-2.69) | 1.27 (0.86-1.87) | 0.046 | 0.210 | 0.221 | 0.119 |
| 85 | SLC12A2 | A | 1.79 (1.20-2.69) | 1.58 (1.14-2.19) | 0.004 | 0.007 | 0.006 | 0.029 |
| 86 | SLC12A2 | A | 1.76 (1.09-2.83) | 1.72 (1.17-2.52) | 0.019 | 0.058 | 0.006 | 0.014 |
| 87 | Intergenic | G | 2.16 (1.06-4.41) | 1.80 (1.06-3.08) | 0.030 | 0.101 | 0.029 | 0.084 |
| 88 | FBN2 | G | 1.81 (1.21-2.69) | 1.49 (1.06-2.10) | 0.004 | 0.012 | 0.020 | 0.073 |
| 89 | Intergenic | A | 1.47 (0.98-2.22) | 1.24 (0.87-1.75) | 0.063 | 0.187 | 0.235 | 0.463 |
| 90 | Intergenic | G | 1.56 (1.05-2.33) | 1.27 (0.90-1.79) | 0.028 | 0.097 | 0.166 | 0.336 |
| 91 | Intergenic | A | 2.19 (1.40-3.41) | 1.72 (1.16-2.53) | 4.92E−04 | 0.002 | 0.006 | 0.021 |
| 92 | ADAMTS19 | G | 1.45 (0.87-2.41) | 1.63 (1.07-2.49) | 0.154 | 0.308 | 0.023 | 0.049 |
| 93 | ADAMTS19/LOC609347 | A | 1.30 (0.78-2.17) | 1.44 (0.94-2.20) | 0.314 | 0.499 | 0.092 | 0.121 |
| 94 | Intergenic | C | 1.81 (1.20-2.74) | 1.37 (0.98-1.92) | 0.005 | 0.018 | 0.064 | 0.162 |
| 95 | Intergenic | A | 1.99 (1.31-3.03) | 1.61 (1.14-2.27) | 0.001 | 0.006 | 0.006 | 0.034 |
| 96 | CHSY3 | A | 1.39 (0.84-2.29) | 1.49 (0.99-2.25) | 0.198 | 0.372 | 0.057 | 0.137 |
| 97 | HAS2 | C | 1.91 (1.09-3.33) | 2.00 (1.28-3.12) | 0.021 | 0.080 | 0.002 | 0.011 |
| 98 | HAS2 | G | 1.31 (0.86-1.99) | 1.44 (1.01-2.04) | 0.210 | 0.456 | 0.044 | 0.135 |
| 99 | COL1A2 | A | 1.74 (1.16-2.63) | 1.72 (1.23-2.41) | 0.008 | 0.013 | 0.001 | 0.001 |
| 100 | IGF1 | G | 1.37 (0.87-2.14) | 1.52 (1.04-2.22) | 0.172 | 0.430 | 0.029 | 0.092 |
| 101 | IGF1 | C | 1.30 (0.84-2.01) | 1.40 (0.96-2.02) | 0.246 | 0.552 | 0.077 | 0.174 |
| 102 | MATN3 | A | 4.22 (0.90-19.76) | 2.48 (0.94-6.51) | 0.048 | 0.045 | 0.058 | 0.054 |
| 103 | near to IL-1A | G | 1.35 (0.78-2.36) | 1.56 (0.98-2.48) | 0.286 | 0.240 | 0.058 | 0.040 |
| 104 | CTSK | A | 1.59 (1.01-2.51) | 1.40 (0.96-2.02) | 0.045 | 0.046 | 0.077 | 0.061 |
| 105 | CTSK | A | 1.52 (0.99-2.33) | 1.17 (0.83-1.65) | 0.053 | 0.005 | 0.370 | 0.002 |
| 106 | CTSD | G | 1.42 (0.92-2.18) | 1.09 (0.77-1.55) | 0.114 | 0.048 | 0.637 | 0.051 |
| 107 | near to PELI3 | A | 1.57 (1.04-2.37) | 1.45 (1.02-2.06) | 0.032 | 0.080 | 0.038 | 0.104 |
| 108 | near to EFEMP2-FBLN4 | A | 1.32 (0.85-2.04) | 1.54 (1.07-2.22) | 0.212 | 0.348 | 0.020 | 0.054 |
| 109 | EFEMP2-FBLN4 | G | 2.00 (1.31-3.06) | 1.82 (1.26-2.63) | 0.001 | 0.006 | 0.001 | 0.008 |
| 110 | EFEMP2-FBLN4 | G | 1.73 (1.15-2.61) | 1.60 (1.13-2.26) | 0.008 | 0.033 | 0.008 | 0.032 |
| 111 | RELA | G | 1.45 (0.95-2.21) | 1.40 (0.98-2.00) | 0.085 | 0.060 | 0.066 | 0.050 |
| 112 | B3GAT3-GLCAT1 | A | * | * | 0.177 | 0.176 | 0.038 | 0.038 |
| 113 | FLNB | A | 3.68 (0.41-33.20) | 8.74 (0.97-78.63) | 0.214 | 0.211 | 0.020 | 0.020 |
| 114 | FLNB | A | 1.39 (0.92-2.09) | 1.30 (0.92-1.83) | 0.117 | 0.037 | 0.131 | 0.120 |
| 115 | COL7A1 | A | 1.12 (0.67-1.87) | 1.12 (0.72-1.73) | 0.677 | 0.041 | 0.611 | 0.275 |
| 116 | near to CCR5 | G | 2.58 (0.81-8.23) | 3.06 (1.21-7.72) | 0.099 | 0.291 | 0.013 | 0.049 |
| 117 | CCR5 | G | 2.60 (0.81-8.30) | 2.73 (1.11-6.69) | 0.095 | 0.285 | 0.023 | 0.062 |
| 118 | near to CCR2 | A | 1.92 (0.93-3.95) | 1.88 (1.06-3.31) | 0.073 | 0.246 | 0.028 | 0.112 |
| 119 | TNA | A | 1.10 (0.62-1.95) | 1.09 (0.68-1.74) | 0.735 | 0.049 | 0.731 | 0.058 |
| 120 | LRCH1 | A | 1.52 (0.91-2.53) | 1.29 (0.83-2.01) | 0.105 | 0.018 | 0.260 | 0.395 |
| 121 | BMP2 | G | 2.94 (1.29-6.70) | 2.11 (1.18-3.76) | 0.008 | 0.040 | 0.010 | 0.039 |
| 122 | BMP2 | G | 1.70 (1.15-2.51) | 1.41 (1.02-1.96) | 0.008 | 0.026 | 0.036 | 0.070 |
| 123 | BMP2 | C | 2.01 (1.21-3.36) | 1.49 (1.01-2.21) | 0.007 | 0.033 | 0.044 | 0.163 |
| 124 | near to GDF5 | G | 1.97 (1.32-2.94) | 1.73 (1.24-2.41) | 0.001 | 0.008 | 0.001 | 0.004 |
| 125 | GDF5 | G | 1.77 (1.18-2.64) | 1.57 (1.12-2.19) | 0.006 | 0.030 | 0.008 | 0.014 |
| 126 | ADAM28 | A | 1.61 (1.07-2.41) | 1.59 (1.14-2.23) | 0.022 | 0.069 | 0.006 | 0.020 |
| 127 | ADAM28 | A | 1.54 (1.02-2.33) | 1.51 (1.08-2.13) | 0.040 | 0.086 | 0.017 | 0.049 |
| 128 | NCOR2 | A | 1.69 (1.07-2.68) | 1.67 (1.15-2.42) | 0.025 | 0.085 | 0.007 | 0.034 |
| 129 | NCOR2 | C | 1.37 (0.88-2.14) | 1.47 (1.02-2.13) | 0.167 | 0.267 | 0.040 | 0.125 |
| 130 | LRP6 | A | 2.18 (1.20-3.95) | 2.05 (1.29-3.26) | 0.009 | 0.046 | 0.002 | 0.006 |
| 131 | LRP6 | C | 1.51 (0.98-2.33) | 1.44 (1.01-2.06) | 0.064 | 0.186 | 0.045 | 0.177 |
| 132 | Intergenic | A | 1.66 (1.09-2.51) | 1.33 (0.93-1.90) | 0.017 | 0.060 | 0.121 | 0.295 |
| 133 | Intergenic | G | 1.43 (0.94-2.20) | 1.27 (0.88-1.82) | 0.098 | 0.216 | 0.200 | 0.324 |
| 134 | VESTIBULE1 | C | 1.80 (1.20-2.72) | 1.46 (1.02-2.07) | 0.005 | 0.014 | 0.036 | 0.119 |
| 135 | VESTIBULE1 | T | 1.66 (1.08-2.56) | 1.42 (1.00-2.02) | 0.020 | 0.051 | 0.050 | 0.156 |
| 136 | VESTIBULE1 | A | 2.18 (1.10-4.33) | 1.88 (1.12-3.17) | 0.023 | 0.059 | 0.016 | 0.041 |
| 137 | VESTIBULE1 | A | 1.87 (1.25-2.81) | 1.54 (1.11-2.15) | 0.002 | 0.020 | 0.010 | 0.027 |
| 138 | VESTIBULE1 | G | 1.83 (1.23-2.73) | 1.56 (1.13-2.17) | 0.003 | 0.026 | 0.007 | 0.025 |
| 139 | VESTIBULE1 | G | 1.82 (1.12-2.93) | 1.73 (1.17-2.56) | 0.014 | 0.071 | 0.006 | 0.030 |
| 140 | VESTIBULE1 | G | 1.77 (1.11-2.81) | 1.47 (1.02-2.13) | 0.016 | 0.052 | 0.040 | 0.008 |
| 141 | Intergenic | G | 1.38 (0.92-2.06) | 1.22 (0.86-1.71) | 0.119 | 0.281 | 0.259 | 0.500 |
| 142 | Intergenic | C | 1.62 (1.07-2.45) | 1.41 (1.01-1.98) | 0.021 | 0.082 | 0.042 | 0.146 |
| 143 | Intergenic | A | 1.51 (1.01-2.27) | 1.28 (0.91-1.82) | 0.045 | 0.148 | 0.156 | 0.263 |
| 144 | Intergenic | A | 1.54 (1.02-2.33) | 1.35 (0.95-1.92) | 0.040 | 0.110 | 0.098 | 0.247 |
| 145 | Intergenic | C | 1.53 (1.01-2.31) | 1.29 (0.91-1.84) | 0.042 | 0.137 | 0.146 | 0.302 |
| 146 | Intergenic | G | 1.54 (1.02-2.33) | 1.32 (0.93-1.89) | 0.040 | 0.110 | 0.118 | 0.288 |
| 147 | SULF1 | T | 1.54 (1.04-2.28) | 1.39 (1.00-1.92) | 0.029 | 0.097 | 0.050 | 0.128 |
| 148 | SULF1 | G | 1.59 (1.01-2.51) | 1.54 (1.07-2.23) | 0.043 | 0.142 | 0.021 | 0.022 |
| 149 | SULF1 | A | 1.67 (1.05-2.66) | 1.52 (1.05-2.21) | 0.030 | 0.092 | 0.026 | 0.013 |
| 150 | SULF1 | G | 1.52 (0.99-2.33) | 1.36 (0.94-1.95) | 0.053 | 0.167 | 0.099 | 0.228 |
| 151 | SULF1 | A | 1.51 (0.98-2.31) | 1.33 (0.93-1.92) | 0.059 | 0.180 | 0.121 | 0.283 |
| 152 | SULF1 | G | 2.20 (1.17-4.12) | 1.77 (1.10-2.84) | 0.012 | 0.082 | 0.017 | 0.093 |
| 153 | SLCO5A1 | A | 1.49 (1.01-2.20) | 1.32 (0.96-1.83) | 0.043 | 0.090 | 0.090 | 0.237 |
| 154 | SLCO5A1 | G | 1.42 (0.95-2.13) | 1.24 (0.88-1.75) | 0.091 | 0.156 | 0.209 | 0.421 |
| 155 | SLCO5A1 | A | 1.97 (1.19-3.25) | 1.45 (0.99-2.13) | 0.007 | 0.023 | 0.057 | 0.206 |
| 156 | SLCO5A1 | A | 1.91 (1.18-3.09) | 1.56 (1.07-2.28) | 0.008 | 0.042 | 0.020 | 0.104 |
| 157 | SLCO5A1 | A | 1.68 (1.13-2.51) | 1.33 (0.95-1.86) | 0.010 | 0.044 | 0.095 | 0.153 |
| 158 | ADAM10 | T | 1.31 (0.89-1.95) | 1.40 (1.01-1.95) | 0.174 | 0.287 | 0.042 | 0.044 |
| 159 | ADAM10 | G | 1.31 (0.88-1.94) | 1.40 (1.01-1.94) | 0.180 | 0.311 | 0.045 | 0.052 |
| 160 | CILP | A | 6.53 (0.80-53.54) | 2.54 (0.84-7.64) | 0.045 | 0.043 | 0.087 | 0.084 |
| 161 | ADAMTS5 | G | 1.12 (0.74-1.70) | 1.24 (0.88-1.76) | 0.584 | 0.198 | 0.223 | 0.045 |
| 162 | near to FRZB | G | 2.14 (0.91-5.03) | 2.13 (1.09-4.14) | 0.076 | 0.147 | 0.023 | 0.012 |
| 163 | ADAM23 | G | 2.44 (1.29-4.60) | 1.79 (1.00-3.19) | 0.005 | 0.015 | 0.047 | 0.114 |
| 164 | ADAM23 | A | 1.92 (1.26-2.95) | 1.64 (1.16-2.30) | 0.002 | 0.010 | 0.004 | 0.007 |
| 165 | ADAM23 | G | 2.09 (1.38-3.18) | 1.85 (1.32-2.60) | 4.82E−04 | 0.001 | 3.18E−04 | 0.001 |

* The allele odds ratio for SNP112 cannot be calculated since there are no individuals for one of the cells of the contingency table.

TABLE 3
Linkage disequilibrium blocks (R² > 0.8) found within the
SNPs associated to canine hip dysplasia and osteoarthritis.

| Chromosome | SNP block | SNP number |
|---|---|---|
| 1 | 1 | 6, 7 |
| | 2 | 11, 12 |
| 3 | 1 | 16, 17, 18 |
| 11 | 1 | 32, 34 |
| | 2 | 36, 37, 38 |
| | 3 | 46, 47, 48 |
| | 4 | 54, 55, 56, 58 |
| | 5 | 60, 61, 62, 63 |
| | 6 | 65, 66, 69 |
| | 7 | 68, 71 |
| | 8 | 70, 72, 73, 74 |
| | 9 | 89, 90 |
| | 10 | 92, 93, 96 |
| 15 | 1 | 100, 101 |
| 17 | 1 | 104, 105 |
| 20 | 1 | 116, 117 |
| 24 | 1 | 124, 125 |
| 25 | 1 | 126, 127 |
| 26 | 1 | 128, 129 |
| 29 | 1 | 132, 133, 134, 141, 143, 144, |
| | | 145, 146, 150, 151 |
| | 2 | 137, 138 |
| | 3 | 148, 149 |
| | 4 | 153, 154 |
| | 5 | 155, 156 |
| 30 | 1 | 158, 159 |
| 37 | 1 | 164, 165 |

The strongest association with CHD and OA was found for two SNPs, 20 and 21, which had been selected as markers for the gene CHST3 (carbohydrate sulfotransferase 3; also named C6ST: chondroitin 6 sulfotransferase). These SNPs were not in LD and showed a strong association with CHD and OA both at allelic and genotypic tests in the two comparisons, A vs DE and AB vs DE (Table 2 A). Canine CHST3 is located on chromosome 4 position: 25902558 to 25905391 (NCBI GeneID: 489036) and contains 2 exons and 1 intron. The SNP20 is located 99 bp upstream of the initial ATG, probably in the putative regulatory promoter region, and the SNP21 is located 1051 bp downstream the gene, likely in the 3′ regulatory region. We did not select SNPs inside CHST3 gene because there were not SNPs described inside the gene in the dog genome databases. The two SNPs selected are the closest SNPs to the 5′ and 3′ ends of the CHST3 gene and were polymorphic both in Labrador retriever and Golden retriever. The closest SNP to the 3′ end of the CHST3 gene, SNP21, was polymorphic in German shepherd dogs.

As shown in Table 4, most of the associations found for CHST3 markers remained significant or borderline (p<0.05) after Bonferroni test correction for multiple comparisons.

TABLE 4
CHST3 markers association with CHD after Bonferroni test correction.

| | | | Chi-squared p | | | |
|---|---|---|---|---|---|---|
| | | Risk | A vs DE | | AB vs DE | |
| SNP | Gene | allele | Allele | Genotype | Allele | Genotype |
| 20 | 5′ upstream | G | 0.012 | 0.164 | 0.033 | 0.399 |
| | CHST3 | | | | | |
| 21 | 3′ downstream | G | 0.010 | 0.052 | 0.002 | 0.005 |
| | CHST3 | | | | | |

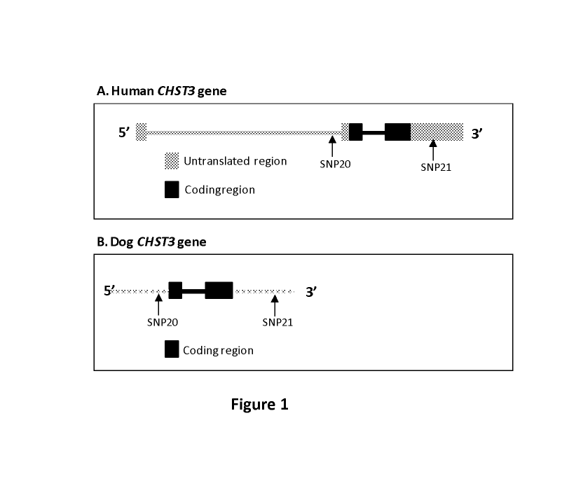

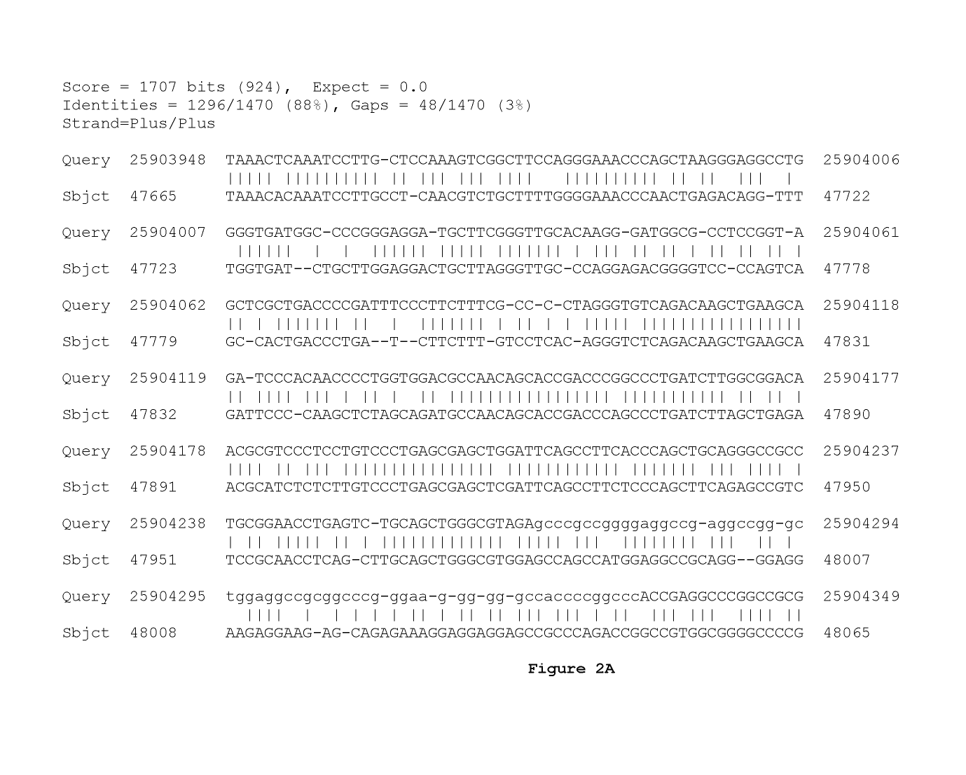

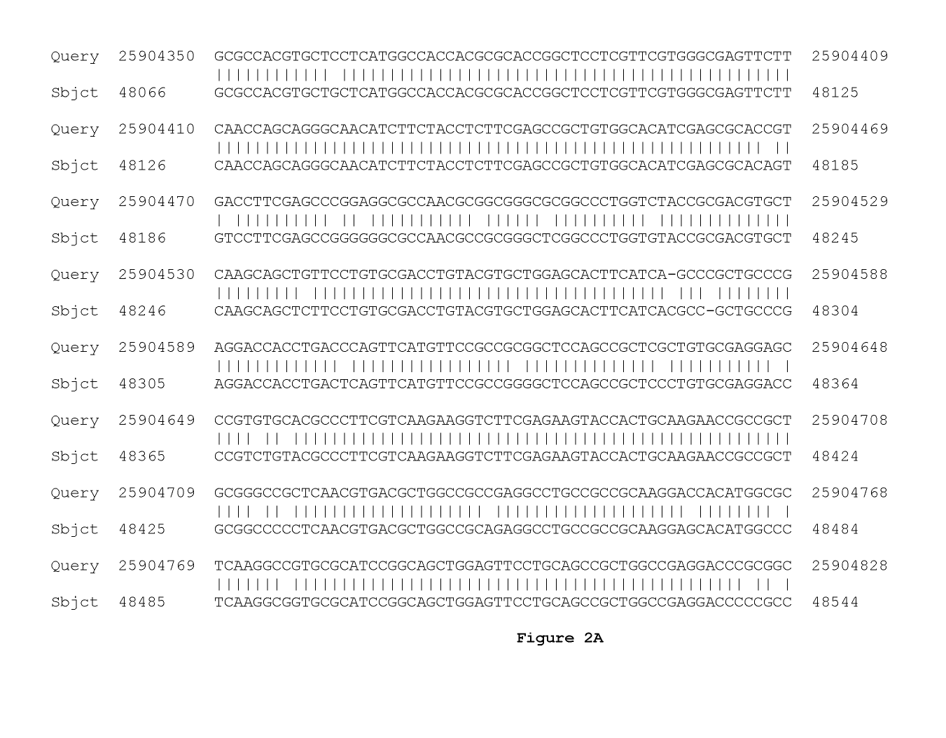

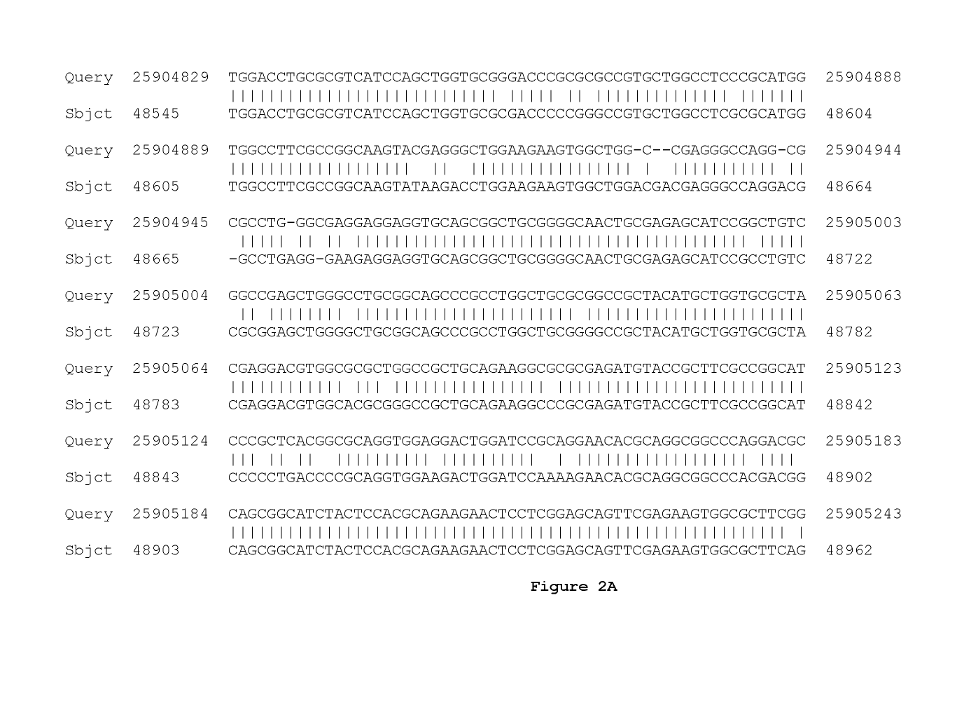

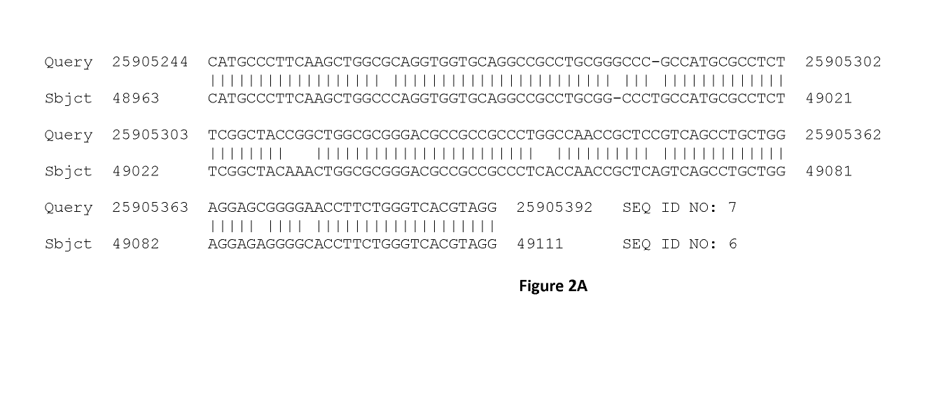

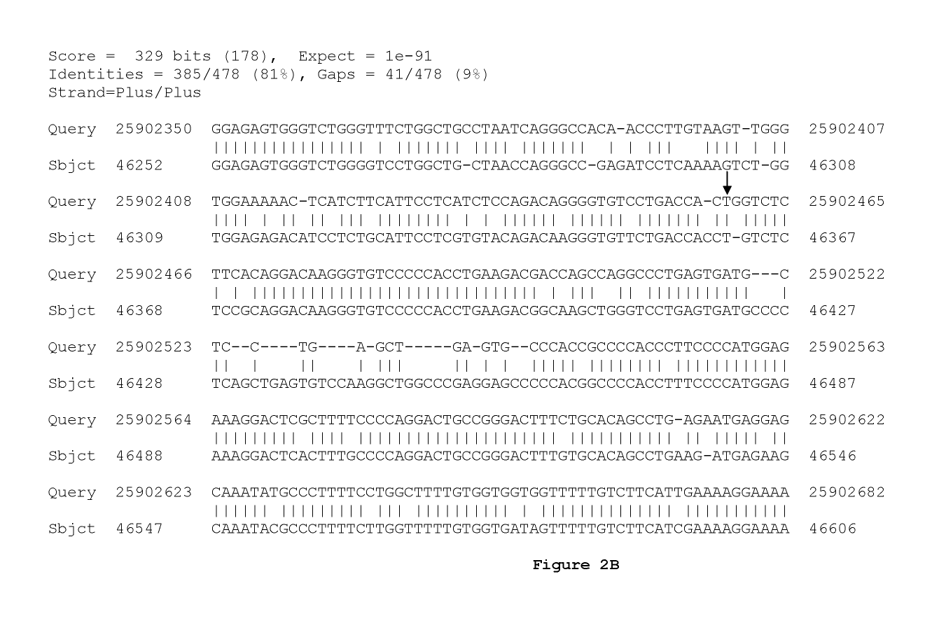

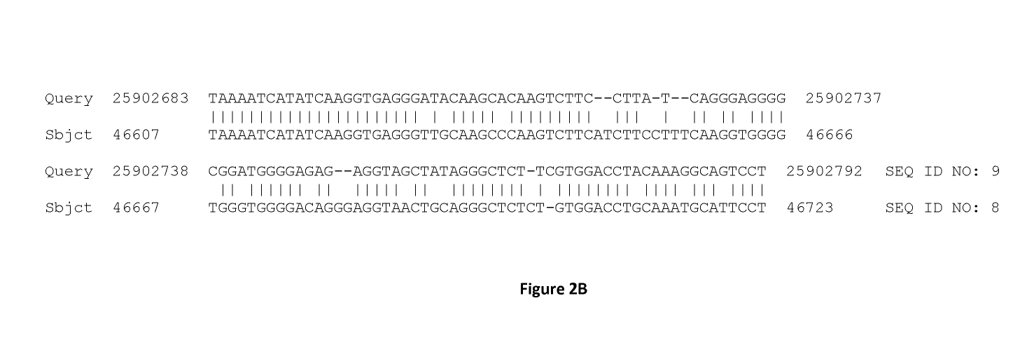

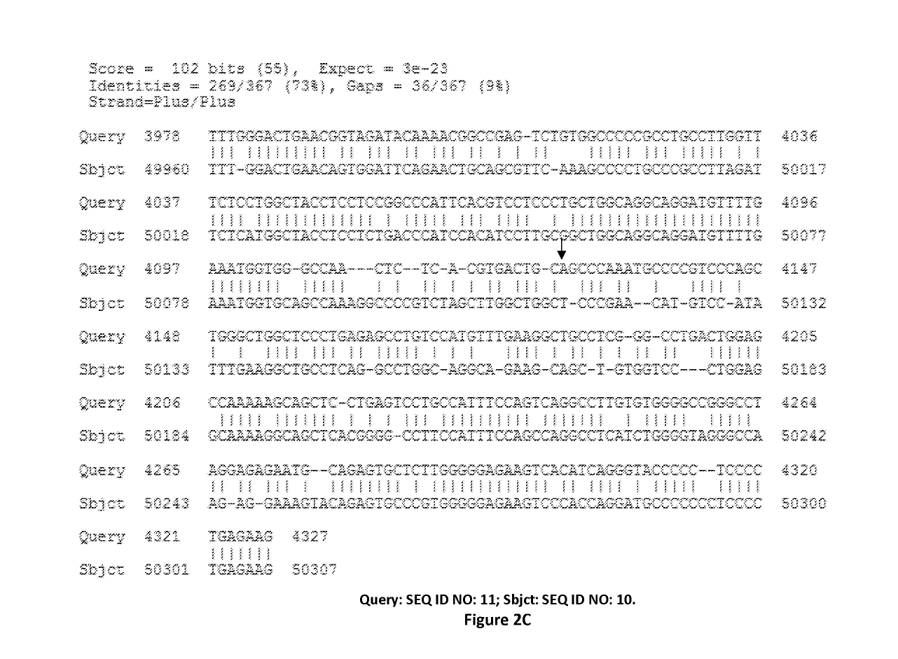

The extension of the 5′ and 3′ regulatory regions of the canine CHST3 gene is not described in the databases of the dog genome (NCBI; CanFam 2.0). The human CHST3 (NCBI GeneID: 9469) gene is located on chromosome 10, is longer than the dog CHST3 gene and the structure of the gene is well-defined (FIG. 1). When comparing the nucleotide sequences of the human and the dog CHST3 genes by means of BLAST (NCBI: BLAST tool) we observed that SNP 20 and 21 are located in regions highly conserved (80% and 73% of identity) between the two species (FIG. 2) (SEQ ID NOs: 6-11). Specifically, the results of the alignment locate the SNP20 in the intron1 (an intron inside the 5′ UTR) and SNP 21 in the 3′ UTR of the human CHST3 gene (FIG. 1). Thus, we could consider that the regions of the dog genome in which SNP20 and 21 are located, regions flanking the CHST3 gene, correspond to the canine CHST3 gene 5′ and 3′ regulatory regions, respectively.

The CHST3 gene encodes an enzyme anchored by its transmembrane domain in the Golgi apparatus and implicated in the biological synthesis of chondroitin sulfate. Chondroitin sulfates are synthesized as proteoglycans that can be expressed on the surfaces of most cells and in extracellular matrices and which are important regulators of many biological processes, such as cell signaling and migration, extracellular matrix deposition, and morphogenesis (Tsutsumi et al. in FEBS Lett. 441, 235-2412-3 (1998); Sugahara et al. in Curr. Opin. Struct. Biol. 10, 518-527 (2000)). Chondroitin sulfate is an important structural component of cartilage and provides much of its resistance to compression. Many of their functions are associated with the sulfation profiles of glycosaminoglycans (GAGs). Chondroitin sulfate has a linear polymer structure that possesses repetitive, sulfated disaccharide units containing glucuronic acid (GlcA) and N-acetylgalactosamine (GalNAc). The major chondroitin sulfate found in mammalian tissues has sulfate groups at position 4 or 6 of GalNac residues (N-acetylgalactosamine). Specifically, CHST3 transfers sulfate groups from 3-phosphoadenosine 5-phosphosulfate (PAPS) and catalyzes sulfation of position 6 of the GalNac, forming chondroitin sulfate 6.

Considering that we found a strong association of the SNPs flanking CHST3 gene with CHD, that CHST3 gene has a relevant role in chondroitin sulfate-6 biosynthesis and that the chondroitin sulfate has an essential function for cartilage biomechanical properties, we sequenced CHST3 gene to search for putative SNPs associated to CHD and OA inside the gene.

We sequenced the CHST3 gene in 39 Labrador retrievers, 20 controls (FCI: A) and 19 cases (15 FCI: E and 4 FCI: D). A fragment including the CHST3 gene and the 5′ upstream (1.7 kb) and 3′ downstream (1.2 kb) regions was amplified by 6 conventional uniplex PCRs. The sequence of the primers used for each PCR is given in Table 5 (SEQ ID NOs: 12-23) and FIG. 3 (SEQ ID NOs: 1 & 4).

TABLE 5
Primers used for PCR amplification of the CHST3 gene
and the 5′ upstream and 3′ downstream regions.

| | | | Amplified | |
|---|---|---|---|---|
| PCR | Primer | Sequence (5′-3′) | region | SEQ ID NO |
| PCR1 | Forward | AGCAGAGAGAGGCTCGAGTG | 5′ upstream | 12 |
| | Reverse | GCCAATCAGCCCTATGATTC | | 13 |
| PCR2 | Forward | GTGCCCAGCCCAGTGCTAAAGG | 5′ upstream + | 14 |
| | Reverse | CCAGAGCCCAAGTGTTATCC | exon1 | 15 |
| PCR3 | Forward | GCTTTTGTGGTGGTGGTTTT | exon1 + intron | 16 |
| | Reverse | CCCATCAGGGTTTGTGTACC | | 17 |
| PCR4 | Forward | AACGATGGGGCTTTCCTTA | intron + exon2 | 18 |
| | Reverse | CCAGCTGCAGACTCAGGTTC | | 19 |
| PCR5 | Forward | TGTCCCGGCTAAACTCAAAT | exon2 + 3′ | 20 |
| | Reverse | CCCACAGTCCCTTCTGGTTA | downstream | 21 |
| PCR6 | Forward | GGCCCAGAACTGTTGACAAG | 3′ downstream | 22 |
| | Reverse | ACAAGGCCTGACTGGAAATG | | 23 |

The PCRs were performed in a 25 μl reaction using the Qiagen Multiplex PCR kit (Qiagen, Hilden, Del.), with a temperature of annealing of 60° C. and with 100 ng of DNA template and 5 pmol of each primer. For PCRs 1, 3, 4, 5 and 6, we added DMSO (8%). PCR products were purified using Millipore HTS filter plates (Millipore, Cork, Ireland). Sequencing reactions of the PCR products were performed with BigDye Terminator v3.1 Cycle Sequencing kit (Applied Biosystemes, USA). Samples were cleaned with CleanSEQ reaction clean-up (Agencourt Bioscience, Beverly, Mass.) and analyzed on an ABI 3100 DNA Analyzer. The sequences of the primers used for sequencing of the two strands, sense and antisense, are given in Table 6 (SEQ ID NOs: 24-57) and FIG. 3 (SEQ ID NOs: 1 & 4).

TABLE 6
Primers used for sequencing of CHST3 gene
and 5′ upstream and 3′ downstream regions.

| | | | SEQ |
|---|---|---|---|
| Primer | Sense | Sequence (5′-3′) | ID NO |
| p1 | Forward | GTGCCCAGCCCAGTGCTAAAGG | 24 |
| p2 | Forward | TGGCATTTGGAATGATCTGA | 25 |
| P3 | Forward | GCTTTTGTGGTGGTGGTTTT | 26 |
| p4 | Forward | TGTCCCGGCTAAACTCAAAT | 27 |
| P5 | Forward | GCGAGTTCTTCAACCAGCAG | 28 |
| P6 | Forward | CAAGTACGAGGGCTGGAAGA | 29 |
| P7 | Forward | GGAACCTTCTGGGTCACGTA | 30 |
| p8 | Forward | CAGAGTCCGAGGCTTAACCA | 31 |
| P9 | Forward | AGCAGAGAGAGGCTCGAGTG | 32 |
| p10 | Forward | GTGCTTAGCTTGGCACCGG | 33 |
| p11 | Forward | CGTGGATGAAGGTCCTTACG | 34 |
| p12 | Forward | ATAGGGCTCTTCGTGGACCT | 35 |
| p13 | Forward | AACGATGGGGCTTTCCTTA | 36 |
| p14 | Forward | GGTTCCTCAGCATGGACTGT | 37 |
| p15 | Forward | GGCCCAGAACTGTTGACAAG | 38 |
| p16 | Forward | ACCAGATTTGGGACTGAAC | 39 |
| p17 | Forward | GCAGTTGAGGCTTTTCAACC | 40 |
| p18 | Forward | TCTCACACACGCACATACACA | 41 |
| p19 | Reverse | CCCACAGTCCCTTCTGGTTA | 42 |
| p20 | Reverse | CCTACGTGACCCAGAAGGTT | 43 |
| p21 | Reverse | GTAGCGCACCAGCATGTAGC | 44 |
| p22 | Reverse | ATGAACTGGGTCAGGTGGTC | 45 |
| p23 | Reverse | CCAGCTGCAGACTCAGGTTC | 46 |
| p24 | Reverse | CCCATCAGGGTTTGTGTACC | 47 |
| p25 | Reverse | CCAGAGCCCAAGTGTTATCC | 48 |
| p26 | Reverse | AGCCAGGAAAAGGGCATATT | 49 |
| p27 | Reverse | ATTCGATCCTGGGTCTCCA | 50 |
| p28 | Reverse | GCCAATCAGCCCTATGATTC | 51 |
| p29 | Reverse | CTCAGCCTCCTGGAGCAG | 52 |
| p30 | Reverse | ATCACACACACCCCTGTCCT | 53 |
| p31 | Reverse | TCCCAGAGGTATCCCTAGCTT | 54 |
| p32 | Reverse | ACAAGGCCTGACTGGAAATG | 55 |
| p33 | Reverse | AAAGCCTCCTCTTTGGGTGT | 56 |
| p34 | Reverse | TGGTGTACGTAGAGGCACTGTC | 57 |

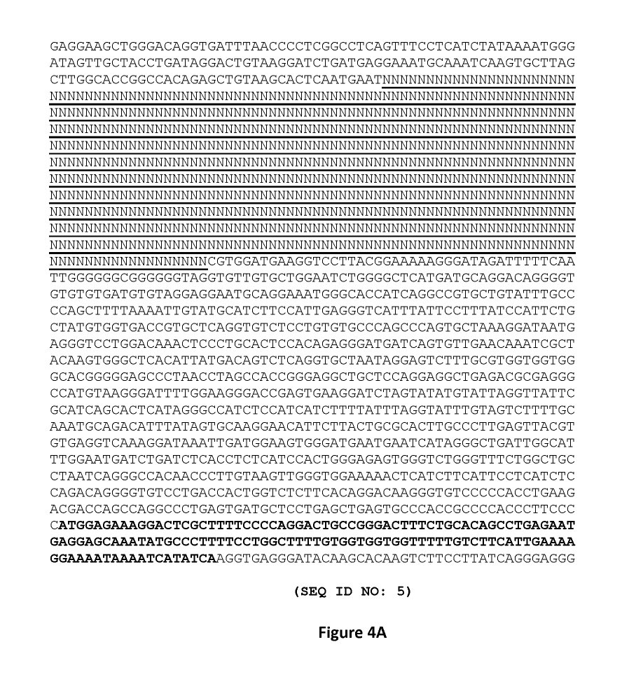

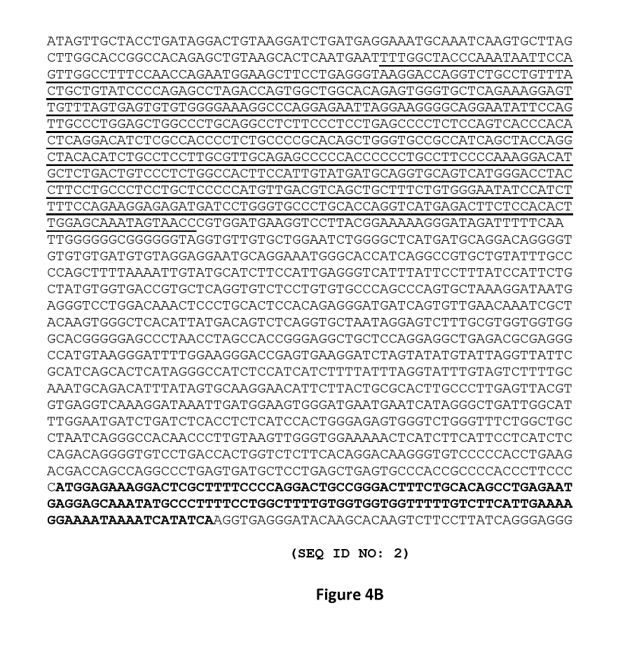

According to the NCBI, there is a gap of 640 bp in the 5′ upstream region of the Boxer Reference sequence of the CHST3 gene (NCBI GeneID: 489036). We sequenced that gap in our 39 Labrador retriever dog cohort and found that the GAP was of 579 bp. The sequence of the gap is shown in FIG. 4 (SEQ ID NO: 2).

We found 37 genetic variants in the dog CHST3 gene and 5′ upstream and 3′ downstream regions (Table 7 and FIG. 5). We detected 31 polymorphic SNPs (including the SNPs 20 and 21 of the Table 1, used as markers for the CHST3 gene and genotyped with Illumina technology), a microsatellite (STR) in the 3′ downstream region, an insertion/deletion (ins/del) in the intron and 4 sequence changes comparing to the Boxer Reference sequence (NCBI: NC_{—}006586.2; Position: 25900817) in the 5′ upstream region. The 4 sequence changes compared to the Boxer reference sequence are the variants C2, C3, C10 and C11 of the Table 7. Three of them are single nucleotide changes and the other one is a change of 3 consecutive nucleotides, compared to the Boxer reference sequence. One of these single nucleotide changes (variant C2 of Table 7) is described as a polymorphic SNP in the boxer sequence and corresponds to the SNP identified as BICF2S23326138 in CanFam 2.0 database. It could be possible that all these monomorphic sequence changes compared to the Boxer reference sequence are, in fact, polymorphic SNPs in Labrador retriever, but with a very small frequency of their minor allele, in such a way that with the small number of dogs (39) sequenced we did not detect the minor allele. The ins/del corresponds to a 187 bp Short Interspersed Nucleotide Element (SINE) previously identified in the Boxer Reference sequence, but not yet described as polymorphic. Polymorphisms of SINE insertions are very common in the dog genome. The STR corresponds to 4, 5 ó 6 repeats of the hexanucleotide sequence TCTCTG and has been previously described in the Boxer Reference sequence.

TABLE 7
Genetic variants found in the CHST3 gene by sequencing of 39 dogs. The allele frequency of each variant
in the 39 dog cohort is shown. The variants are displayed in order of appearance in the sequence.

| Genetic variant | Location | SEQ ID NO | Type of variant | Allele frequency | |
|---|---|---|---|---|---|
| C1 | 5′ upstream | 58 | AAAATGGGAT[A/C]GTTGCTACCT | f(A): 0.88 | f(C): 0.12 |
| C2 (BICF2S23326138) | 5′ upstream | 59 | GTTGCTACCT[G/A]ATAGGACTGT | f(G): 0 | f(A): 1 |
| C3 | 5′ upstream | 60 | AGCACTCAAT[G/A]AATTTTGGCT | f(G): 0 | f(A): 1 |
| C4 | 5′ upstream | 61 | TTAGGAAGGG[G/A]CAGGAATATT | f(G): 0.91 | f(A): 0.09 |
| C5 | 5′ upstream | 62 | CCCCTCTCCA[G/A]TCACCCACAC | f(G): 0.88 | f(A): 0.12 |
| C6 | 5′ upstream | 63 | CCCTCTGCCC[C/T]GCACAGCTGG | f(C): 0.96 | f(T): 0.04 |
| C7 | 5′ upstream | 64 | AGCTGGGTGC[C/T]GCCATCAGCT | f(C): 0.92 | f(T): 0.08 |
| C8 | 5′ upstream | 65 | GAGCCCCCAC[C/T]CCCCTGCCTT | f(C): 0.55 | f(T): 0.45 |
| C9 | 5′ upstream | 66 | CTTCCATTGT[A/G]TGATGCAGGT | f(A): 0.56 | f(G): 0.44 |
| C10 | 5′ upstream | 67 | GGCGGGGGGT[A/G]GGTGTTGTGC | f(A): 0 | f(G): 1 |
| C11 | 5′ upstream | 68, 69 | GTGTGTGATG[TGT/GTG]AGGAGGA | f(TGT): 0 | f(GTG): 1 |
| C12 (BICF2P772451) | 5′ upstream | 70 | AAACTCCCTG[C/A]ACTCCACAGA | f(C): 0.91 | f(A): 0.09 |
| C13 | 5′ upstream | 71 | GTGGGCTCAC[A/G]TTATGACAGT | f(A): 0.55 | f(G): 0.45 |
| C14 | 5′ upstream | 72 | GGAAGGGACC[G/A]AGTGAAGGAT | f(G): 0.58 | f(A): 0.42 |
| C15 (BICF2P772452) | 5′ upstream | 73 | TCTCCATCAT[C/T]TTTTATTTAG | f(C): 0.35 | f(T): 0.65 |
| C16 (BICF2P772453) | 5′ upstream | 74 | TCTTACTGCG[C/T]ACTTGCCCTT | f(C): 0.91 | f(T): 0.09 |
| C17 (BICF2P772454) | 5′ upstream | 75 | CTCACCTCTC[A/T]TCCACTGGGA | f(A): 0.35 | f(T): 0.65 |
| C18 (SNP20 of | 5′ upstream | 76 | GTCCTGACCAC[T/C]GGTCTCTTCA | f(T): 0.52 | f(C): 0.48 |
| Table 1: BICF2P772455) | | | | | |
| C19 | Intron | 77 | CAGGGAGGGG[C/T]GGATGGGGAG | f(C): 0.88 | f(T): 0.12 |
| C20 | Intron | | Ins/Del of a SINE (187 bp) | f(del). 0.67 | f(ins): 0.33 |
| C21 | Intron | 78 | ATAAAAAAAA[A/T]AAAAAAAAAA | f(A): 0.78 | f(T): 0.22 |
| C22 | Intron | 79 | AGTGGGCCTG[C/T]ACAGGTCCTC | f(C): 0.34 | f(T): 0.66 |
| C23 | Intron | 80 | TGCACAGGTC[C/T]TCAGGACTAC | f(C): 0.29 | f(T): 0.71 |
| C24 | Intron | 81 | CCACCCCCTG[G/A]AGGTGGCATT | f(A): 0.12 | f(G): 0.88 |
| C26 | Intron | 82 | GACTGTTCCA[G/C]TTGGGGCCCA | f(G): 0.54 | f(C): 0.46 |
| C27 | Intron | 83 | GGGAGCAGCC[C/T]TTAGCTAAGA | f(C): 0.55 | f(T): 0.45 |
| C28 | Intron | 84 | AGACAATCCT[C/T]GGGTGTGCCC | f(C): 0.89 | f(T): 0.11 |
| C29 | Intron | 85 | AGACAATCCTC[G/A]GGTGTGCCC | f(A): 0.08 | f(G): 0.92 |
| C30 | Intron | 86 | CGGGAGGATG[C/T]TTCGGGTTGC | f(C): 0.88 | f(T): 0.12 |
| C31 | Exon2 (Leu52Leu) | 87 | CAGACAAGCT[G/A]AAGCAGATCC | f(A): 0.18 | f(G): 0.76 |
| C32 | Exon2 (Arg118Gly) | 88 | GGCCGCGGCC[C/G]GGGAAGGGGG | f(G): 0.24 | f(C): 0.76 |
| C33 | Exon2 (Ala180A1a) | 89 | GCGCCAACGC[G/C]GCGGGCGCGG | f(G): 0.90 | f(C): 0.10 |
| C34 | Exon2 (Leu214Leu) | 90 | AGGACCACCT[G/C]ACCCAGTTCA | f(G): 0.74 | f(C): 0.26 |
| C35 | 3′ downstream | 91-93 | STR (TCTCTG)₄₋₅₋₆ | | |
| C36 | 3′ downstream | 94 | ACAGAGCTAC[G/A]AAACACACCT | f(A): 0.21 | f(G): 0.79 |
| C37 | 3′ downstream | 95 | AGATACAAAA[C/T]GGCCGAGTC | f(C): 0.97 | f(T): 0.03 |
| C38 (SNP21 of | 3′ downstream | 96 | ACGTGACTGC[A/G]GCCCAAATGC | f(A): 0.90 | f(G): 0.10 |
| Table 1 BICFP419109) | | | | | |

*Considering the Ilumina′s nomenclature for DNA strand identification, the allele T of the genetic variant C18 (SNP20 of Table 1) corresponds to the allele A of the TOP strand and the allele C to the allele G of the TOP strand.

Six of the 31 SNPs detected in the CHST3 gene, and located in the 5′ upstream and 3′ downstream regions, had been previously described for Boxer in the CanFam 2.0 database (Table 7): C12 (BICF2P772451), C15 (BICF2P772452), C16 (BICF2P772453), C17 (BICF2P772454), C18 (BICF2P772455) and C38 (BICFP419109). The other 25 SNPs are new SNPs not previously described in the CHST3 gene for any dog breed. Nine of them are located in the 5′ upstream region which could correspond to the putative promoter (C1, C4, C5, C6, C7, C8, C9, C13, C14), 10 in the intron (C19, C21, C22, C23, C24, C25, C26, C27, C28, C30), 4 in the exon2 (C31, C32, C33, C34) and 2 in the 3′ downstream regulatory region (C36, C37). Three of the SNPs of exon2 are synonymous SNPs, Leu52Leu, Ala180Ala and Leu214 Leu (NCBI protein Reference Sequence: XP_{—}546154.1). The other one is a non-synonymous SNP resulting in an arginine to glycine exchange, Arg118Gly. This is a non-conservative exchange which substitutes a negatively charge residue, Arg, with a non-charge residue, Gly.

We found that some of the SNPs of the CHST3 gene were in strong linkage disequilibrium (r²>0.8). The SNPs within a same LD block are shown in Table 8.

TABLE 8
Linkage disequilibrium blocks (R² > 0.8) found within
the SNPs in the CHST3 in the population of 39 dogs.

| SNP block | SNP number |
|---|---|
| 1 | C1-C19-C24-C30-C33 |
| 2 | C4-C7-C12-C16 |
| 3 | C8-C13-C26-C27 |
| 4 | C9-C14 |
| 5 | C15-C17 |
| 6 | C31-C36 |
| 7 | C32-C34 |

Once identified the genetic variants inside the CHST3 gene and in its flanking regions, we performed an association test with CHD and OA for allele and genotype frequencies in the sub-population of 39 dogs sequenced. Based on the p-value of the results, we selected 17 SNPs for genotyping in the whole population of dogs in search of an association with CHD and OA. The SNPs selected were the variants: C6, C12, C13, C14, C15, C16, C17, C19, C22, C23, C29, C30, C31, C32, C33, C34 and C36 of the Table 8.

All the SNPs, except the SNP C32 (Table 7), were genotyped using the KASPar chemistry (KBioscience, Hertfordshire, UK), which is a competitive allele specific PCR SNP genotyping system using FRET quencher cassette oligonucleotides. The SNP C32 was genotyped by using polymerase chain reaction-restriction fragment length polymorphism (PCR-RFLP) technique. The presence of one of the alleles of the SNP (allele G) alters a blunt restriction site for SmaI enzyme (site:CCC/GGG). The PCRs were performed in a 25 μl reaction using the Qiagen Multiplex PCR kit (Qiagen, Hilden, Del.), with a temperature of annealing of 60° C. and with 100 ng of DNA template and 5 pmol of each primer. The primers used for PCR amplification are shown in FIG. 6 (SEQ ID NOs: 20 & 44). Besides the restriction site altered by the SNP C32, the fragment amplified by PCR contains another restriction site for SmaI.

We performed allele and genotype association tests for the 17 SNPs genotyped in the CHST3 gene considering the following groups: A vs DE. We found that in addition to the SNPs C18 (SNP 20 of Table 1) and C38 (SNP21 of Table 1), 7 additional SNPs (C6, C15, C17, C23, C32, C34 and C36) in the CHST3 gene were associated to CHD and OA in the allele or genotype association test (Table 9). Five of these 7 SNPs (C6, C23, C32, C34 and C36) were new SNPs described for the first time in the CHST3 gene. The most significant associations, after the SNPs C18 and C38, were found for the non-synonymous-SNP Arg118Gly (C32) and the synonymous SNP Leu214Leu (C34), previously found to be in LD in the sub-population of 39 dogs. We also performed the allele and genotype association tests for the 17 SNPs comparing AB vs DE dogs and we did not find any significant change respect to the results obtained in the A vs DE comparison (Table 9). These results point out the CHST3 gene as an important gene contributing to CHD and OA genetic predisposition and to diseases secondary to CHD.

TABLE 9
Results of the allele and genotype association analysis with CHD and
OA (A vs DE) for the novel SNPs in the CHST3 gene. SNPs were
genotyped by competitive allele-specific PCR or PCR-RFLP.

| | Chi squared p | | | |
|---|---|---|---|---|
| | Risk | Odds ratio allele | | |
| Genetic variant (SNP) | allele | OR (95% CI) | Allele | Genotype |
| C34 | C | 2.24 (1.35-3.71) | 1.50E−03 | 4.51E−03 |
| C32 | G | 2.20 (1.33-3.62) | 1.71E−03 | 3.21E−03 |
| C36 | G | 1.82 (1.06-3.12) | 0.027 | 0.095 |
| C17 (BICF2P772454) | T | 1.54 (1.04-2.27) | 0.030 | 0.040 |
| C15 (BICF2P772452) | T | 1.53 (1.04-2.26) | 0.031 | 0.025 |
| C6 | T | 7.76 (0.93-64.99) | 0.025 | 0.138 |
| C23 | T | 1.42 (0.97-2.09) | 0.072 | 0.049 |
| C31 | G | 1.66 (0.98-2.83) | 0.058 | 0.188 |
| C14 | A | 1.35 (0.92-1.99) | 0.126 | 0.317 |
| C13 | G | 1.28 (0.86-1.91) | 0.228 | 0.438 |
| C22 | T | 1.25 (0.83-1.88) | 0.272 | 0.465 |
| C16 (BICF2P772453) | T | 1.35 (0.74-2.44) | 0.324 | 0.614 |
| C12 (BICF2P772451) | A | 1.28 (0.86-1.91) | 0.416 | 0.713 |
| C29 | A | 1.24 (0.72-2.12) | 0.435 | 0.871 |
| C19 | T | 1.22 (0.58-2.50) | 0.604 | 0.866 |
| C33 | C | 1.25 (0.51-3.02) | 0.624 | 0.626 |
| C30 | T | 1.15 (0.57-2.33) | 0.690 | 0.879 |

We analyzed the LD pattern (r²>0.8) of the 17 SNPs genotyped in the whole population (475), considering also the SNPs 20 and 21. The SNPs within a same LD block are shown in Table 10. We found that the LD blocks observed with the sub-population of 39 dogs were maintained when the analysis was performed in the whole population of dogs.

TABLE 10
Linkage disequilibrium blocks (R² > 0.8) found within
the SNPs in the CHST3 gene in the population of 475 dogs.

| SNP block | SNP number |
|---|---|
| 1 | C12, C16, C19 |
| 2 | C13, C22 |
| 3 | C15, C17 |
| 4 | C31, C36 |
| 5 | C32, C34 |
| 6 | C19, C30 |

Besides SNP C18 (SNP 20 of Table 1) and C38 (SNP 21 of Table 1), 11 of the 17 SNPs analyzed in the CHST3 gene were also polymorphic in Golden retriever (n=18) (Table 11). The SNP C38 (BICF2P419109) was analyzed by PCR-RFLP. The presence of one of the alleles of the SNP (allele G) alters a cohesive restriction site for PstI enzyme (site: CTGCA/G). The PCRs were performed in a 25 μl reaction using the Qiagen Multiplex PCR kit (Qiagen, Hilden, Del.), with a temperature of annealing of 60° C. and with 100 ng of DNA template and 5 pmol of each primer. The primers used for PCR amplification are shown in FIG. 7 (SEQ ID NOs: 40 & 55).

TABLE 11
Genotype frequency of the 17 SNPs analyzed in the CHST3 gene in
Golden retriever (n = 18) and German shepherd * (n = 23)
dogs The number of dogs with each genotype is shown in brackets.

| | |
|---|---|
| SNP number | Genotypes |

| | | | |
|---|---|---|---|
| C18 | G_G(10) | A_G(7) | A_A(1) |
| C38 | G_G(8) | A_G(7) | A_A(2) |
| C38* | G_G(15) | A_G(6) | A_A(2) |
| C6 | C_C(17) | C_T(0) | T_T(0) |
| C29 | G_G(1) | A_G(8) | A_A(9) |
| C12 | C_C(1) | A_C(8) | A_A(9) |
| C13 | A_A(16) | A_G(1) | G_G(0) |
| C14 | G_G(17) | A_G(1) | A_A(0) |
| C15 | C_C(1) | T_C(7) | T_T(10) |
| C16 | C_C(1) | T_C(8) | T_T(9) |
| C17 | A_A(1) | T_A(7) | T_T(10) |
| C19 | C_C(18) | T_C(0) | T_T(0) |
| C22 | C_C(17) | T_C(1) | C_C(0) |
| C23 | C_C(1) | T_C(7) | T_T(10) |
| C30 | C_C(18) | T_C(0) | T_T(0) |
| C31 | G_G(18) | A_G(0) | A_A(0) |
| C32 | C_C(17) | C_G(1) | G_G(0) |
| C33 | G_G(18) | G_C(0) | C_C(0) |
| C34 | G_G(17) | G_C(1) | C_C(0) |
| C36 | G_G(18) | A_G(0) | A_A(0) |

The CHST3 gene, which we found associated to CHD and OA, has not been previously described as associated to canine hip dysplasia or OA and it is not included inside any of the QTLs found by other authors to be linked to canine HD or OA.

We analyzed if any of the clinical variables, coat colour, adult weight, birth weight, gender, age, exercise habits, diet habits, usual type of floor, early spay, mortality before weaning and litter size was associated to CHD. We found an association for coat color in Labrador retrievers. Dogs with a yellow color have a higher predisposition to CHD than chocolate or black dogs (p=0.006).

Regarding to the GWAS strategy, the Labrador retrievers graded as A, B, D and E were genotyped using the Illumina's Canine HD BeadChip (Illumina Inc., San Diego, Calif.) which includes more than 170,000 evenly spaced and validated SNPs derived from the CanFam 2.0 assembly. A total of 240 Labrador retrievers were analyzed separately into two groups, 129 controls (A and B) and 111 cases (D and E). We applied quality control at both individual and SNP levels and some samples and markers were subsequently excluded (call rate <99%, minor allele frequency <0.01 or Hardy-Weinberg equilibrium p>1×10⁻⁴ in controls). The final population of dogs consisted of 227 Labrador retrievers (122 controls and 105 cases) genotyped for a total of 139433 SNPs. An association test, A+B (controls) vs D+E (cases), was performed to identify the SNPs associated to CHD and OA. After false discovery rate (FDR) correction for multiple testing, 250 SNPs remained significantly associated to CHD (p<1.96×10⁻⁵) (Table 12 A, B, C and D).

TABLE 12
SNPs found in the GWAS to be associated to canine hip dysplasia and osteoarthritis. SNP code
according to CanFam 2.0 database, chromosome position, nucleotide change and the chi-squared
p and odds ratio of the risk allele considering Illumina's TOP strand nomenclature are shown.

| SNP | | | nt | Risk | | | Chi- | |
|---|---|---|---|---|---|---|---|---|
| number | SNP code | Gene | change | allele | Chr | nt position | squared p | OR (95% CI) |
| 166 | BICF2S2327284 | ME2 | A/G | A | 1 | 27055561 | 8.95E−02 | 3.6 (1.8-6.9) |
| 167 | BICF2G630720098 | PHACTR2 | C/A | C | 1 | 38298960 | 5.85E−03 | 3.1 (1.9-5.2) |
| 168 | BICF2G630720115 | PHACTR2 | A/G | A | 1 | 38328623 | 4.34E−03 | 3.1 (1.9-5.1) |
| 169 | BICF2S23763633 | RCL1 | G/A | G | 1 | 96255702 | 3.97E−02 | 2.9 (1.7-4.9) |
| 170 | BICF2P770991 | near to ZNF114, CYTH2, SNRP70 | G/A | G | 1 | 110857650 | 1.82E−03 | 2.6 (1.7-3.8) |
| 171 | BICF2P1002269 | near to ZNF114 | A/G | A | 1 | 110872054 | 2.15E−03 | 2.5 (1.7-3.8) |
| 172 | BICF2P161177 | LIG1/LR1G1 | A/G | A | 1 | 111004523 | 2.21E−03 | 2.6 (1.7-3.8) |
| 173 | BICF2P1446055 | CABP5 | G/A | G | 1 | 111040923 | 2.21E−03 | 2.6 (1.7-3.8) |
| 174 | BICF2P478505 | ELSPBP1 | G/A | G | 1 | 111047122 | 2.21E−03 | 2.6 (1.7-3.8) |
| 175 | BICF2P931526 | GLTSCR1 | G/A | G | 1 | 111233845 | 1.69E−02 | 3.2 (1.8-5.4) |
| 176 | BICF2P612416 | near to KIF17, TNSALP(ALPL) | G/A | G | 2 | 81285897 | 1.28E−02 | 2.7 (1.7-4.3) |
| 177 | BICF2P876960 | TAS1R2 | A/G | A | 2 | 82632256 | 1.85E−05 | 3.3 (1.9-5.9) |
| 178 | BICF2P1310360 | ARSK | A/G | A | 3 | 16885023 | 3.65E−02 | 2.4 (1.6-3.6) |
| 179 | BICF2P1395755 | near to FAM81B | A/G | A | 3 | 17070803 | 2.36E−03 | 2.8 (1.8-4.4) |
| 180 | BICF2S23324938 | near to EDIL3 | G/A | G | 3 | 25630966 | 2.96E−02 | 2.9 (1.7-4.7) |
| 181 | BICF2P618822 | near to EDIL3 | A/C | A | 3 | 26212315 | 7.83E−03 | 2.8 (1.7-4.4) |
| 182 | BICF2P525869 | near to EDIL3 | G/A | G | 3 | 26245187 | 1.03E−02 | 2.8 (1.7-4.6) |
| 183 | BICF2P906090 | EDIL3 | A/G | A | 3 | 26634672 | 1.26E−02 | 2.6 (1.7-3.9) |
| 184 | BICF2S23450252 | near to EDIL3, HAPLN1, | G/A | G | 3 | 26760791 | 3.51E−02 | 2.6 (1.6-4.1) |
| | | CSPG2/VCAN | | | | | | |
| 185 | BICF2S23097 | SSBP2 | A/C | A | 3 | 28758077 | 5.56E−05 | 3.1 (2.1-4.6) |
| 186 | BICF2S2355183 | ACOT12 | A/G | A | 3 | 28849658 | 5.34E−04 | 2.6 (1.8-3.9) |
| 187 | BICF2P664113 | arsb-Q32K14 | A/G | A | 3 | 30774041 | 5.20E−03 | 3.2 (1.9-5.3) |
| 188 | BICF2G630704471 | arsb-Q32K14 | G/A | G | 3 | 30782110 | 2.17E−02 | 2.8 (1.7-4.6) |
| 189 | BICF2G630704490 | arsb-Q32K14 | A/G | A | 3 | 30799265 | 9.88E−03 | 2.7 (1.7-4.2) |
| 190 | BICF2G630706365 | C5orf37 | A/G | A | 3 | 33605155 | 1.35E−06 | 4.7 (2.4-9.2) |
| 191 | BICF2G630106037 | OCA2 | G/A | G | 3 | 35288824 | 4.36E−03 | 4.4 (2.2-8.7) |
| 192 | BICF2S23327759 | near to GABRA5 | G/A | G | 3 | 36076169 | 2.11E−03 | 2.9 (1.8-4.5) |
| 193 | BICF2G630106489 | near to GABRA5 | G/A | G | 3 | 36328333 | 1.82E−06 | 4.8 (2.4-9.7) |
| 194 | BICF2P889439 | near to GABRA5 | C/A | C | 3 | 36330130 | 5.28E−03 | 2.6 (1.7-3.9) |
| 195 | BICF2G630106615 | near to GABRA5 | A/C | A | 3 | 36476262 | 2.12E−03 | 3.7 (2.1-6.5) |
| 196 | BICF2G630106625 | near to GABRA5 | A/G | A | 3 | 36482789 | 7.14E−04 | 4.5 (2.4-8.4) |
| 197 | BICF2G630106677 | GABRA5 | G/A | G | 3 | 36530531 | 2.00E−04 | 4.5 (2.4-8.1) |
| 198 | BICF2G630106693 | GABRA5 | A/C | A | 3 | 36542297 | 2.00E−04 | 4.5 (2.4-8.1) |
| 199 | BICF2P424215 | GABRA5 | G/A | G | 3 | 36548895 | 8.81E−03 | 2.6 (1.7-4.1) |
| 200 | BICF2G630106718 | near to GABRA5 | A/C | A | 3 | 36633140 | 4.83E−03 | 2.7 (1.7-4.1) |
| 201 | BICF2G630106786 | near to GABRA5 | A/G | A | 3 | 36738612 | 1.29E−03 | 2.7 (1.8-4.1) |
| 202 | BICF2G630106787 | near to GABRA5 | A/C | A | 3 | 36748479 | 1.32E−03 | 2.7 (1.8-4.1) |
| 203 | BICF2G630107204 | near to GABRA3 | G/A | G | 3 | 37297566 | 1.34E−03 | 2.7 (1.8-4.1) |
| 204 | TIGRP2P41503_rs8599393 | ATP10A | G/A | G | 3 | 37766813 | 2.44E−06 | 3.5 (2.1-5.9) |
| 205 | BICF2P579846 | near to NDN | G/A | G | 3 | 39202443 | 2.34E−03 | 3.4 (2.1-5.8) |
| 206 | BICF2P156516 | OTUD7A | A/G | A | 3 | 40270774 | 2.97E−07 | 5.3 (2.7-10.6) |
| 207 | BICF2P684982 | near to MCEE | G/A | G | 3 | 41048247 | 2.61E−06 | 2.8 (1.8-4.3) |
| 208 | BICF2G630338747 | near to MCEE | A/G | A | 3 | 41063353 | 9.88E−04 | 3.7 (2.1-6.3) |
| 209 | BICF2G630338879 | APBA2 | C/A | C | 3 | 41251838 | 5.72E−03 | 2.6 (1.7-3.9) |
| 210 | BICF2G630339399 | near to PC5K6, TM2D3, CHSY1 | G/A | G | 3 | 42404899 | 2.17E−03 | 2.7 (1.7-4.1) |
| 211 | BICF2G630339806 | near to ADAMTS17, MEF2A, SYNM | A/G | A | 3 | 43159521 | 5.03E−04 | 7.4 (3.1-18.3) |
| 212 | BICF2G630340881 | near to ARRDC4, IGF1R | C/G | C | 3 | 45808638 | 1.20E−04 | 4.2 (2.4-7.4) |
| 213 | BICF2G630340902 | near to ARRDC4 | A/G | A | 3 | 45820255 | 5.34E−06 | 3.6 (2.1-6.5) |
| 214 | BICF2G630340909 | near to ARRDC4 | A/G | A | 3 | 45831376 | 2.64E−04 | 4.0 (2.3-6.9) |
| 215 | BICF2G630340916 | near to ARRDC4 | G/A | G | 3 | 45844780 | 1.72E−03 | 3.6 (2.1-6.2) |
| 216 | BICF2S23447407 | NTRK3 | G/A | G | 3 | 54142311 | 1.85E−03 | 3.0 (1.9-4.7) |
| 217 | BICF2P236884 | NTRK3 | G/A | G | 3 | 54202813 | 3.06E−03 | 2.7 (1.7-4.1) |
| 218 | BICF2G63058908 | near to KCNK1, TARBP1 | A/G | A | 4 | 9307356 | 1.39E−04 | 7.9 (3.3-19.3) |
| 219 | BICF2G63058940 | near to KCNK1 | G/A | G | 4 | 9336983 | 5.63E−04 | 4.0 (2.7-7.1) |
| 220 | BICF2G63058969 | MLK4-MAP3K19 | A/T | A | 4 | 9371926 | 1.39E−04 | 7.9 (3.3-19.3) |
| 221 | BICF2G63059021 | near to PCNXL2 | G/A | G | 4 | 9419399 | 5.06E−07 | 5.8 (2.7-12.3) |
| 222 | BICF2G63059130 | PCNXL2 | A/G | A | 4 | 9544627 | 1.00E−03 | 5.0 (2.5-9.9) |
| 223 | BICF2G63059131 | PCNXL2 | G/A | G | 4 | 9545169 | 1.00E−03 | 5.0 (2.5-9.9) |
| 224 | BICF2P440076 | near to BICC1, PHYHIPL | G/A | G | 4 | 14426721 | 5.03E−04 | 7.4 (3.1-18.1) |
| 225 | BICF2S23513308 | ANK3 | A/G | A | 4 | 15574320 | 3.15E−04 | 4.1 (2.3-7.3) |
| 226 | TIGRP2P58893_rs9244440 | near to ANK3 | A/G | A | 4 | 15749280 | 1.74E−02 | 3.9 (2.1-7.6) |
| 227 | BICF2P1090418 | near to ANK3 | A/T | A | 4 | 15763010 | 7.17E−03 | 4.0 (2.1-7.6) |
| 228 | BICF2P1128397 | CBARA1 | A/G | A | 4 | 26247141 | 8.04E−02 | 0.5 (0.3-0.7) |
| 229 | BICF2P648799 | near to KCMA1_CANFA, DLG5 | A/C | A | 4 | 29963744 | 3.81E−03 | 0.4 (0.3-0.6) |
| 230 | BICF2P466720 | near to SH2D4B | A/T | A | 4 | 32989534 | 2.61E−02 | 2.2 (1.5-3.3) |
| 231 | BICF2S2412468 | near to GHITM | C/A | C | 4 | 35169727 | 2.23E−04 | 7.0 (3.1-16.1) |
| 232 | BICF2P676099 | near to GHR_CANFA | G/A | G | 4 | 70523477 | 1.36E−03 | 3.5 (2.1-5.9) |
| 233 | BICF2P815932 | near to GHR_CANFA | G/A | G | 4 | 70539446 | 2.26E−03 | 3.0 (1.9-4.9) |
| 234 | BICF2P761581 | C7 | A/T | A | 4 | 71695867 | 1.05E−03 | 2.6 (1.7-3.7) |
| 235 | BICF2P235645 | near to C7 | A/G | A | 4 | 71771551 | 2.30E−03 | 4.2 (2.2-7.9) |
| 236 | BICF2P910266 | near to TTC33 | G/A | G | 4 | 71998209 | 1.90E−04 | 3.5 (2.1-5.6) |
| 237 | BICF2P1018431 | near to TTC33 | A/G | A | 4 | 72004044 | 1.57E−03 | 3.4 (2.1-5.7) |
| 238 | BICF2P243838 | near to TTC33 | G/A | G | 4 | 72015406 | 8.75E−06 | 3.1 (1.8-5.2) |
| 239 | BICF2P563602 | near to TTC33 | A/G | A | 4 | 72176979 | 2.33E−02 | 2.4 (1.6-3.6) |
| 240 | BICF2P1144701 | near to TTC33 | G/A | G | 4 | 72266068 | 9.60E−03 | 2.5 (1.6-3.7) |
| 241 | BICF2P1009099 | near to TTC33 | G/A | G | 4 | 72291658 | 1.74E−02 | 3.9 (2.1-7.6) |
| 242 | BICF2P236590 | near to DAB2 | A/G | A | 4 | 72481509 | 2.01E−03 | 2.5 (1.7-3.6) |
| 243 | BICF2P347050 | near to DAB2 | A/G | A | 4 | 72487665 | 2.01E−03 | 2.5 (1.7-3.6) |
| 244 | BICF2P1358015 | near to DAB2 | G/A | G | 4 | 72493191 | 3.48E−03 | 2.4 (1.7-3.5) |
| 245 | BICF2S2377318 | near to LIFR_CANFA, RICTOR | A/G | A | 4 | 73485664 | 2.04E−06 | 3.3 (1.9-5.4) |
| 246 | BICF2P785116 | near to LIFR_CANFA | A/C | A | 4 | 73585389 | 6.12E−05 | 2.5 (1.6-3.9) |
| 247 | TIGRP2P64964_rs8872527 | EGFLAM | A/G | A | 4 | 73612129 | 4.34E−03 | 3.1 (1.9-5.1) |
| 248 | BICF2G630168456 | NIPBL | A/G | A | 4 | 74853932 | 3.16E−03 | 3.1 (1.9-5.1) |
| 249 | BICF2P1040220 | near to NIPBL | A/G | A | 4 | 75070474 | 1.37E−05 | 4.1 (2.1-8.1) |
| 250 | BICF2P1330558 | near to TNR | A/G | A | 7 | 26726207 | 1.48E−02 | 2.5 (1.6-3.7) |
| 251 | BICF2G630558118 | near to HNRNPU | A/G | A | 7 | 38913207 | 7.83E−03 | 2.8 (1.7-4.4) |
| 252 | BICF2G630558172 | near to HNRNPU | A/C | A | 7 | 39010182 | 1.28E−02 | 2.7 (1.7-4.3) |
| 253 | BICF2G630558226 | near to KIF26B | G/A | G | 7 | 39115202 | 5.09E−02 | 2.4 (1.6-3.7) |
| 254 | BICF2G630558235 | near to KIF26B | C/A | C | 7 | 39131807 | 5.63E−03 | 2.6 (1.7-3.9) |
| 255 | BICF2G630558239 | near to KIF26B | A/G | A | 7 | 39142101 | 7.81E−07 | 3.6 (2.1-6.1) |
| 256 | BICF2G630558272 | near to KIF26B | A/G | A | 7 | 39162063 | 5.28E−02 | 2.2 (1.5-3.3) |
| 257 | BICF2S23764774 | near to SMYD3, CDC42BPA | G/A | G | 7 | 40126824 | 3.03E−05 | 2.3 (1.5-3.3) |
| 258 | TIGRP2P96127_rs8947528 | near to FCRL4, HAPLN2, MEF2D, | A/G | A | 7 | 43648368 | 1.31E−02 | 2.3 (1.6-3.4) |
| | | BCAN, HDGF, NDPP1-CARD8, | | | | | | |
| | | BGLAP | | | | | | |
| 259 | BICF2S23026364 | near to DLGAP1, EMILIN2, MYL12A, | G/A | G | 7 | 73997252 | 4.40E−03 | 0.4 (0.3-0.6) |
| | | MYL12B, MYOM1 | | | | | | |
| 260 | BICF2G63087113 | near to ACAA2, LIPG, DYM, SPIRE1 | A/G | A | 7 | 82005777 | 1.94E−02 | 2.7 (1.7-4.3) |
| 261 | BICF2P601580 | near to LEG3, PELI2, BMP4 | C/G | C | 8 | 33990753 | 1.81E−05 | 0.4 (0.3-0.6) |
| 262 | BICF2S23733435 | near to SEL1L | G/A | G | 8 | 57237774 | 1.69E−05 | 2.8 (1.7-4.4) |
| 263 | TIGRP2P117749_rs8995490 | near to SEL1L | C/A | C | 8 | 57391860 | 3.51E−02 | 2.6 (1.6-4.1) |
| 264 | BICF2P1278239 | near to SEL1L | A/G | A | 8 | 58682525 | 2.33E−02 | 2.4 (1.6-3.6) |
| 265 | BICF2S23616305 | near to FLRT2 | G/A | G | 8 | 59131555 | 3.57E−02 | 2.4 (1.6-3.6) |
| 266 | BICF2P589325 | PPP4R4 | A/G | A | 8 | 66292143 | 9.80E−03 | 3.0 (1.8-5.1) |
| 267 | TIGRP2P118734_rs9140055 | PPP4R4 | A/G | A | 8 | 66338160 | 9.90E−04 | 3.3 (1.9-5.3) |
| 268 | BICF2P1193152 | near to BCL11B | G/C | G | 8 | 69854157 | 3.57E−05 | 2.9 (1.7-4.9) |
| 269 | BICF2P900262 | near to BCL11B | G/A | G | 8 | 70071624 | 2.30E−03 | 3.2 (1.9-5.3) |
| 270 | BICF2P349191 | near to BCL11B | G/A | G | 8 | 70087189 | 4.38E−02 | 2.8 (1.7-4.7) |
| 271 | BICF2P428480 | ZNF385C | A/G | A | 9 | 24142916 | 1.35E−06 | 4.7 (2.4-9.2) |
| 272 | BICF2P660167 | ZNF385C | A/G | A | 9 | 24159667 | 6.36E−03 | 3.5 (1.9-6.2) |
| 273 | BICF2G630830616 | near to KRT26, ELN | A/C | A | 9 | 25251644 | 9.96E−03 | 4.1 (2.1-7.8) |
| 274 | BICF2G630830621 | KRT26 | A/G | A | 9 | 25256521 | 9.96E−03 | 4.1 (2.1-7.8) |
| 275 | BICF2P1420892 | near to CA10, COL1A1 | G/A | G | 9 | 30970175 | 9.82E−05 | 2.1 (1.4-3.1) |
| 276 | BICF2P146712 | near to APPL2, TXNRD1, SEPT10, | C/A | C | 10 | 36123523 | 1.22E−03 | 3.1 (1.9-5.1) |
| | | CHST11 | | | | | | |
| 277 | BICF2S23036428 | near to SIX2 | A/G | A | 10 | 50585703 | 4.59E−03 | 3.3 (1.9-5.5) |
| 278 | BICF2P879346 | near to SIX2 | G/A | G | 10 | 50601178 | 1.08E−02 | 2.6 (1.7-4.1) |
| 279 | BICF2S23426994 | near to SRBD1 | G/A | G | 10 | 50729667 | 2.97E−03 | 2.5 (1.7-3.6) |
| 280 | TIGRP2P140889_rs8627994 | near to SRBD1 | G/A | G | 10 | 50767357 | 4.50E−03 | 2.4 (1.7-3.6) |
| 281 | TIGRP2P140899_rs8880524 | near to SRBD1 | G/A | G | 10 | 50801676 | 4.50E−03 | 2.4 (1.7-3.6) |
| 282 | BICF2P1429720 | near to SRBD1 | G/A | G | 10 | 50805618 | 4.50E−03 | 2.4 (1.7-3.6) |
| 283 | BICF2P1229357 | SRBD1 | C/A | C | 10 | 50989016 | 4.37E−03 | 3.5 (2.1-6.1) |
| 284 | BICF2P138204 | SRBD1 | G/A | G | 10 | 50999225 | 4.37E−03 | 3.5 (2.1-6.1) |
| 285 | TIGRP2P140920_rs8563734 | SRBD1 | A/G | A | 10 | 51003990 | 4.37E−03 | 3.5 (2.1-6.1) |
| 286 | BICF2P1324352 | SRBD1 | G/A | G | 10 | 51027235 | 4.37E−03 | 3.5 (2.1-6.1) |
| 287 | TIGRP2P140942_rs8957933 | SRBD1 | G/A | G | 10 | 51047274 | 1.00E−03 | 2.6 (1.7-3.8) |
| 288 | BICF2S2452559 | SRBD1 | G/A | G | 10 | 51049130 | 6.16E−06 | 4.5 (2.6-7.7) |
| 289 | BICF2P278101 | SLC1A4 | G/A | G | 10 | 67637494 | 6.87E−05 | 4.1 (2.4-7.1) |
| 290 | BICF2S23251761 | SLC1A4 | A/G | A | 10 | 67647447 | 5.28E−03 | 3.0 (1.8-4.8) |
| 291 | BICF2P526962 | near to SLC1A4, CEP68 | G/A | G | 10 | 67660908 | 3.32E−04 | 3.4 (2.1-5.5) |
| 292 | BICF2P823840 | near to RAB1A_CANFA | G/A | G | 10 | 67803124 | 4.76E−03 | 2.9 (1.8-4.5) |
| 293 | BICF2P1336575 | SPRED2 | A/C | A | 10 | 67922240 | 2.05E−03 | 4.0 (2.2-7.3) |
| 294 | TIGRP2P153295_rs9164620 | near to TNC, TNFS15, FKBP15 | A/G | A | 11 | 72302465 | 3.71E−02 | 2.3 (1.5-3.5) |
| 295 | TIGRP2P158316_rs9164582 | near to GPR110 | G/A | G | 12 | 18210492 | 1.45E−03 | 3.7 (2.1-6.4) |
| 296 | BICF2S23357027 | near to GPR110 | A/G | A | 12 | 18261191 | 5.34E−06 | 3.6 (2.1-6.5) |
| 297 | BICF2P795047 | near to GPR110 | A/C | A | 12 | 18380086 | 8.61E−04 | 3.1 (1.9-4.9) |
| 298 | BICF2P941307 | near to GPR110 | A/G | A | 12 | 18441810 | 3.32E−02 | 2.6 (1.6-4.2) |
| 299 | BICF2P1097570 | CD2AP | A/G | A | 12 | 18651283 | 2.03E−04 | 3.2 (2.1-5.2) |
| 300 | TIGRP2P158471_rs8951942 | near to GPR111 | A/G | A | 12 | 18711775 | 9.54E−03 | 2.9 (1.8-4.7) |
| 301 | BICF2P548082 | near to OPN5 | G/A | G | 12 | 19413721 | 1.28E−02 | 2.7 (1.7-4.3) |
| 302 | BICF2P1397736 | near to MUT | A/T | A | 12 | 20219734 | 2.66E−06 | 3.0 (1.8-4.7) |
| 303 | BICF2S23652446 | near to MUT | A/G | A | 12 | 20307857 | 2.26E−03 | 3.0 (1.9-4.9) |
| 304 | TIGRP2P159391_rs8698534 | near to RHAG | A/G | A | 12 | 20500042 | 6.58E−05 | 2.2 (1.5-3.2) |
| 305 | BICF2P841785 | near to C6orf142, TRAM2 | A/G | A | 12 | 24505091 | 4.09E−02 | 2.2 (1.5-3.2) |
| 306 | BICF2S23056118 | near to ABRA | A/G | A | 13 | 10755023 | 8.44E−02 | 2.3 (1.5-3.5) |
| 307 | BICF2S23417189 | CDK6 | A/G | A | 14 | 21273908 | 5.99E−03 | 2.5 (1.7-3.7) |
| 308 | BICF2P336597 | ICA1 | G/A | G | 14 | 26636900 | 2.30E−03 | 3.2 (1.9-5.3) |
| 309 | BICF2P787552 | near to XM_532481,2 (ningun gen) | A/C | A | 14 | 32814269 | 5.90E−03 | 4.5 (2.3-9.1) |
| 310 | BICF2P927953 | near to HERPUD2, SEPTINE7 | A/G | A | 14 | 50105430 | 2.87E−04 | 31.4 (4.2-233.9) |
| 311 | BICF2S22914443 | neear to GPR141 | G/A | G | 14 | 51616166 | 8.17E−05 | 4.2 (2.4-7.3) |
| 312 | BICF2S23447436 | neear to GPR141 | G/A | G | 14 | 51628310 | 8.29E−04 | 3.8 (2.2-6.6) |
| 313 | BICF2P305876 | near to SCMH1, COL9A2, NFYC, | A/G | A | 15 | 4890205 | 1.25E−02 | 3.9 (2.1-7.3) |
| | | ZMPSTE24 | | | | | | |
| 314 | TIGRP2P194884_rs8705005 | near to GJB5 | G/A | G | 15 | 10233587 | 3.08E−02 | 3.6 (1.9-6.7) |
| 315 | TIGRP2P194963_rs8923342 | near to GJB5 | A/G | A | 15 | 10453765 | 3.33E−02 | 2.4 (1.6-3.7) |
| 316 | BICF2P283225 | near to CSMD2, ANXA2 | A/G | A | 15 | 10465905 | 5.14E−03 | 2.6 (1.7-4.1) |
| 317 | BICF2G630444326 | C16orf87 | A/C | A | 15 | 11320580 | 4.49E−02 | 3.2 (1.8-5.8) |
| 318 | BICF2G630443770 | NRD1 | A/C | A | 15 | 12459204 | 2.71E−02 | 2.7 (1.7-4.3) |
| 319 | BICF2S23748144 | FAM160A1 | G/A | G | 15 | 52568364 | 2.24E−02 | 2.4 (1.6-3.7) |
| 320 | BICF2P351020 | near to C4orf18 | A/G | A | 15 | 58129192 | 8.94E−03 | 3.8 (2.1-7.1) |
| 321 | BICF2P1451267 | KCNQ1 | A/G | A | 18 | 49632449 | 1.27E−02 | 0.4 (0.3-0.6) |
| 322 | BICF2S2303264 | near to ANO1, CTTN | A/G | A | 18 | 51280258 | 7.45E−04 | 0.3 (0.2-0.5) |
| 323 | BICF2P472851 | near to ANO1 | G/C | G | 18 | 51293436 | 2.71E−02 | 0.4 (0.3-0.6) |
| 324 | BICF2S23029139 | near to FGF3 | A/G | A | 18 | 51315017 | 6.42E−03 | 0.4 (0.3-0.6) |
| 325 | BICF2S230609 | near to TPCN2, LRP5 | G/A | G | 18 | 51721581 | 4.17E−07 | 0.3 (0.2-0.5) |
| 326 | BICF2P766553 | LRP1B | A/G | A | 19 | 46364546 | 2.93E−02 | 2.7 (1.7-4.3) |
| 327 | BICF2P1336956 | near to LRP1B | A/G | A | 19 | 46974751 | 9.40E−03 | 2.4 (1.6-3.6) |
| 328 | TIGRP2P268225_rs8813006 | near to LRP1B | G/A | G | 19 | 46987648 | 3.28E−03 | 2.9 (1.8-4.5) |
| 329 | TIGRP2P268234_rs9104397 | near to LRP1B | A/G | A | 19 | 46996173 | 5.74E−03 | 2.8 (1.7-4.4) |
| 330 | BICF2S23354263 | near to KYNU | C/A | C | 19 | 47921517 | 9.30E−03 | 2.5 (1.7-3.7) |
| 331 | BICF2P619851 | KYNU | A/C | A | 19 | 48143919 | 4.80E−03 | 3.0 (1.8-4.9) |
| 332 | BICF2P65006 | ARHGAP15 | A/G | A | 19 | 48667266 | 5.12E−03 | 3.4 (1.9-5.9) |
| 333 | BICF2G630227898 | RAB7A_CANFA | A/G | A | 20 | 5707575 | 1.55E−04 | 4.6 (2.5-8.6) |
| 334 | BICF2G630227914 | RAB7A_CANFA | G/A | G | 20 | 5720949 | 3.53E−02 | 2.8 (1.7-4.5) |
| 335 | BICF2P527689 | RAB7A_CANFA | G/A | G | 20 | 5738027 | 3.53E−02 | 2.8 (1.7-4.5) |
| 336 | BICF2G630227933 | RAB7A_CANFA | A/G | A | 20 | 5741533 | 3.53E−02 | 2.8 (1.7-4.5) |
| 337 | BICF2G630227941 | RAB7A_CANFA | A/G | A | 20 | 5752627 | 3.53E−02 | 2.8 (1.7-4.5) |
| 338 | BICF2G630227965 | RAB7A_CANFA | A/G | A | 20 | 5771454 | 3.53E−02 | 2.8 (1.7-4.5) |
| 339 | BICF2G630227973 | near to RAB7A_CANFA | A/G | A | 20 | 5779740 | 3.53E−02 | 2.8 (1.7-4.5) |
| 340 | BICF2G630227985 | near to H1FX, FBLN2 | G/A | G | 20 | 5790816 | 3.53E−02 | 2.8 (1.7-4.5) |
| 341 | BICF2P598981 | near to FAM19A4, FRMD4B, LMOD3 | C/G | C | 20 | 25809154 | 6.44E−02 | 3.2 (1.8-5.9) |
| 342 | BICF2P612540 | near to FAM19A4 | G/A | G | 20 | 26314118 | 2.36E−02 | 2.8 (1.7-4.5) |
| 343 | BICF2P173460 | near to SLC25A26, MAGI1 | A/C | A | 20 | 28465749 | 4.59E−03 | 3.3 (1.9-5.5) |
| 344 | BICF2P485140 | near to SLC25A26, MAGI1 | A/G | A | 20 | 28525002 | 5.65E−06 | 4.2 (2.2-8.1) |
| 345 | BICF2P580416 | PTPRG | A/C | A | 20 | 31844266 | 4.67E−04 | 0.4 (0.2-0.5) |
| 346 | BICF2P642325 | PTPRG | A/G | A | 20 | 31851278 | 9.08E−06 | 0.4 (0.3-0.6) |
| 347 | BICF2S23123519 | PTPRG | A/G | A | 20 | 31856031 | 2.24E−03 | 0.4 (0.3-0.6) |
| 348 | BICF2S23217200 | PTPRG | C/A | C | 20 | 31875137 | 9.76E−05 | 0.3 (0.2-0.5) |
| 349 | BICF2P837085 | PTPRG | G/A | G | 20 | 31913386 | 3.30E−07 | 3.0 (1.9-4.7) |
| 350 | BICF2P595868 | near to PTPRG | A/G | A | 20 | 32089221 | 8.35E−03 | 2.8 (1.7-4.4) |
| 351 | BICF2S23551778 | near to C3orf67, FLNB | G/A | G | 20 | 34238966 | 1.94E−02 | 2.5 (1.6-3.9) |
| 352 | TIGRP2P296196_rs8820470 | near to FGF14 | C/A | C | 22 | 54600325 | 6.49E−06 | 2.4 (1.6-3.5) |
| 353 | BICF2P619290 | near to ERCC5 | G/A | G | 22 | 55722758 | 2.37E−02 | 2.4 (1.6-3.6) |
| 354 | BICF2S2294860 | near to CLRN1 | G/A | G | 23 | 48563627 | 1.00E−02 | 2.3 (1.6-3.4) |
| 355 | BICF2G630368262 | near to CLRN1 | G/A | G | 23 | 48693729 | 2.25E−03 | 2.6 (1.7-3.9) |
| 356 | BICF2P1026855 | near to MBLN1 | G/A | G | 23 | 50172563 | 6.80E−02 | 3.0 (1.7-5.2) |
| 357 | BICF2P60416 | THOC5 | A/G | A | 26 | 25794542 | 3.79E−02 | 2.2 (1.5-3.2) |
| 358 | BICF2G630408751 | near to ATP8B4, CEP152, | A/G | A | 30 | 19098020 | 4.12E−02 | 2.2 (1.5-3.2) |
| | | FGF7_CANFA | | | | | | |
| 359 | TIGRP2P369146_rs8776891 | near to SLC27A2, HDC | G/A | G | 30 | 19126099 | 4.07E−03 | 4.1 (2.2-7.7) |
| 360 | BICF2S23021949 | near to GLDN, CYP19 | A/G | A | 30 | 20035089 | 1.30E−05 | 3.1 (1.8-5.3) |
| 361 | BICF2P295156 | near to GLDN | A/G | A | 30 | 20050399 | 5.85E−03 | 3.1 (1.9-5.2) |
| 362 | BICF2G630408521 | GLDN | A/G | A | 30 | 20112510 | 3.47E−03 | 3.2 (1.9-5.3) |
| 363 | BICF2G630401339 | ZNF609 | C/A | C | 30 | 31978450 | 1.35E−03 | 2.6 (1.7-3.9) |
| 364 | BICF2G630401334 | ZNF609 | A/G | A | 30 | 31990257 | 1.35E−03 | 2.6 (1.7-3.9) |
| 365 | BICF2G630401283 | near to ZNF609 | T/A | T | 30 | 32112415 | 2.18E−03 | 2.5 (1.7-3.8) |
| 366 | BICF2G630401151 | PLEKHO2 | A/C | A | 30 | 32257905 | 1.70E−02 | 0.4 (0.3-0.6) |
| 367 | BICF2P464939 | near to C3orf38 | A/G | A | 33 | 3053413 | 1.21E−03 | 0.4 (0.3-0.6) |
| 368 | BICF2S23628331 | near to EPHA3 | A/G | A | 33 | 3499170 | 5.58E−04 | 2.6 (1.8-3.8) |
| 369 | BICF2S2323286 | near to EPHA3 | G/A | G | 33 | 3670157 | 5.29E−03 | 2.4 (1.6-3.5) |
| 370 | BICF2S23711437 | near to EPHA3 | T/A | T | 33 | 3720337 | 1.88E−03 | 2.5 (1.7-3.7) |
| 371 | BICF2P1145835 | near to EPHA3 | G/A | G | 33 | 3763500 | 1.75E−03 | 2.5 (1.7-3.7) |
| 372 | BICF2G630244778 | near to NSUN3 | G/A | G | 33 | 4998177 | 1.46E−04 | 2.7 (1.9-4.1) |
| 373 | BICF2G630244789 | near to NSUN3 | C/A | C | 33 | 5007731 | 2.52E−04 | 2.7 (1.8-3.9) |
| 374 | TIGRP2P390878_rs9092335 | near to NSUN3 | A/G | A | 33 | 5014530 | 5.61E−05 | 3.0 (1.9-4.4) |
| 375 | BICF2P1136726 | near to NSUN3 | G/C | G | 33 | 5773355 | 8.46E−03 | 0.4 (0.3-0.6) |
| 376 | BICF2G630245484 | near to NSUN3 | G/A | G | 33 | 5999290 | 1.64E−03 | 2.5 (1.7-3.7) |
| 377 | BICF2P839475 | near to NSUN3 | A/G | A | 33 | 6028101 | 5.62E−03 | 2.4 (1.6-3.5) |
| 378 | BICF2G630245491 | near to NSUN3 | G/A | G | 33 | 6163962 | 7.73E−03 | 0.4 (0.3-0.6) |
| 379 | BICF2P1321188 | near to EPHA6 | A/C | A | 33 | 7398844 | 2.47E−02 | 0.4 (0.3-0.6) |
| 380 | BICF2P903863 | near to EPHA6 | G/A | G | 33 | 7430038 | 1.53E−02 | 0.4 (0.3-0.6) |
| 381 | BICF2G630245754 | near to EPHA6 | G/A | G | 33 | 7468432 | 2.47E−02 | 0.4 (0.3-0.6) |
| 382 | BICF2G630245758 | near to EPHA6 | A/G | A | 33 | 7484346 | 2.47E−02 | 0.4 (0.3-0.6) |
| 383 | BICF2G630246506 | near to DCBLD2, FILIP1L | G/A | G | 33 | 8967429 | 1.52E−05 | 2.4 (1.6-3.6) |
| 384 | BICF2P1007883 | near to DCBLD2, FILIP1L | C/A | C | 33 | 8971284 | 9.60E−03 | 2.5 (1.6-3.7) |
| 385 | BICF2G630246514 | near to DCBLD2, FILIP1L | A/G | A | 33 | 8983975 | 5.99E−03 | 2.5 (1.7-3.7) |
| 386 | BICF2G630246943 | near to TBC1D23, FILIP1L | G/A | G | 33 | 9835180 | 2.03E−02 | 0.4 (0.3-0.6) |
| 387 | BICF2G630249309 | near to CD166_CANFA | A/G | A | 33 | 12996689 | 8.38E−04 | 2.7 (1.8-4.1) |
| 388 | TIGRP2P385957_rs9049307 | near to CD166_CANFA | G/A | G | 33 | 13127715 | 5.94E−05 | 2.9 (1.9-4.3) |
| 389 | BICF2S23515275 | near to CD166_CANFA | A/G | A | 33 | 14008613 | 8.83E−02 | 0.5 (0.3-0.7) |
| 390 | BICF2P995251 | near to CD166_CANFA | A/G | A | 33 | 14021633 | 4.08E−03 | 0.4 (0.3-0.6) |
| 391 | BICF2P345479 | near to CD166_CANFA | G/A | G | 33 | 14125278 | 3.52E−05 | 2.2 (1.5-3.3) |
| 392 | BICF2S23546726 | near to CD166_CANFA | G/A | G | 33 | 14155820 | 8.83E−02 | 0.5 (0.3-0.7) |
| 393 | BICF2P458854 | near to CCDC52 | A/G | A | 33 | 20863581 | 1.51E−03 | 3.2 (1.9-5.3) |
| 394 | BICF2G63080325 | ZBTB20 | G/A | G | 33 | 21609217 | 5.04E−07 | 3.2 (2.1-5.1) |
| 395 | BICF2G63080318 | near to ZBTB20 | A/G | A | 33 | 21665280 | 2.36E−02 | 2.8 (1.7-4.5) |
| 396 | BICF2G630452632 | near to GOLIM4 | G/A | G | 34 | 35947639 | 3.28E−03 | 2.9 (1.8-4.5) |
| 397 | BICF2P909639 | near to GMDS | A/T | A | 35 | 5872757 | 1.39E−03 | 2.6 (1.7-3.9) |
| 398 | TIGRP2P410898_rs8604820 | near to GMDS | C/A | C | 35 | 5892924 | 5.51E−06 | 2.4 (1.6-3.7) |
| 399 | BICF2P189633 | near to WRNIP1, MYLK4 | A/G | A | 35 | 6078049 | 1.94E−02 | 2.7 (1.7-4.3) |
| 400 | BICF2P644389 | near to PECI | G/A | G | 35 | 7458108 | 1.23E−05 | 15.0 (4.5-49.6) |
| 401 | BICF2P1045684 | F13A1 | A/G | A | 35 | 9258327 | 3.46E−03 | 3.0 (1.8-4.9) |
| 402 | BICF2P787863 | F13A1 | C/A | C | 35 | 9297028 | 6.28E−03 | 2.9 (1.8-4.7) |
| 403 | BICF2P1242205 | NRP2 | C/A | C | 37 | 17299306 | 2.57E−03 | 3.2 (1.9-5.2) |
| 404 | BICF2P1084334 | near to FAM5C | G/A | G | 38 | 10637459 | 4.80E−03 | 3.0 (1.8-4.9) |
| 405 | BICF2P1176600 | near to MARK1 | A/C | A | 38 | 18213078 | 1.52E−02 | 0.4 (0.3-0.6) |
| 406 | BICF2G630534598 | near to MAGBA_CANFA | G/A | G | X | 23459332 | 9.16E−02 | 3.9 (1.9-8.1) |
| 407 | BICF2G630534587 | near to MAGBA_CANFA | A/C | A | X | 23471978 | 1.96E−04 | 3.6 (1.8-7.3) |
| 408 | BICF2G630533696 | near to GK | G/A | G | X | 25667974 | 2.17E−03 | 2.2 (1.3-3.6) |
| 409 | BICF2G630533695 | near to CXorf21, MAP3K71P3/TAB3 | G/A | G | X | 25668666 | 2.17E−03 | 2.2 (1.3-3.6) |
| 410 | BICF2G630533005 | near to Q6Q275_CANFA | G/A | G | X | 27282330 | 1.48E−04 | 2.5 (1.5-3.9) |
| 411 | BICF2G630532995 | near to Q6Q275_CANFA | A/G | A | X | 27298479 | 9.24E−02 | 2.5 (1.5-3.9) |
| 412 | BICF2G630532991 | near to Q6Q275_CANFA | G/A | G | X | 27305579 | 1.47E−04 | 2.5 (1.5-3.9) |
| 413 | BICF2G63011475 | near to SHT2C | C/A | C | X | 89807098 | 2.26E−04 | 2.5 (1.5-4.1) |
| 414 | BICF2G63011418 | near to SHT2C | A/G | A | X | 89898656 | 6.37E−04 | 3.2 (1.6-6.3) |
| 415 | BICF2G63010276 | PGRMC1 | G/A | G | X | 94401132 | 1.04E−02 | 3.5 (1.9-6.4) |

We analyzed the linkage disequilibrium pattern of the SNPs found to be associated to CHD and OA in the GWAS. We found several blocks in different chromosomes (Table 13 A, B and C). It was also investigated if any of the SNPs found to be associated to CHD and OA in the candidate gene strategy was in LD with the SNPs found in the GWAS. We observed that the SNP 8 (BICF2S23036087) from the candidate gene strategy (Table 1 and 2) was in LD with several SNPs from the GWAS in the same chromosome (Chr 1).

TABLE 13

| | |
|---|---|
| SNP code (CanFam 2.0) | Chromosome |

A. Linkage disequilibrium blocks (R² > 0.8) found within
the SNPs associated to CHD in the GWAS (Table 12) and
in the candidate gene strategy (Table 2). The SNPs in
linkage disequilibrium are in the same box.

| | |
|---|---|
| BICF2G630227914 | 20 |
| BICF2G630227933 | |
| BICF2G630227941 | |
| BICF2G630227965 | |
| BICF2G630227973 | |
| BICF2G630227985 | |
| BICF2P527689 | |
| SNP 8 (BICF2S23036087) | 1 |
| BICF2P1002269 | |
| BICF2P1446055 | |
| BICF2P161177 | |
| BICF2P478505 | |
| BICF2P770991 | |
| BICF2P1229357 | 10 |
| BICF2P1324352 | |
| BICF2P138204 | |
| BICF2S2452559 | |
| TIGRP2P140920_rs8563734 | |
| BICF2P1429720 | 10 |
| BICF2S23426994 | |
| TIGRP2P140889_rs8627994 | |
| TIGRP2P140899_rs8880524 | |
| TIGRP2P140942_rs8957933 | |
| BICF2G630245754 | 33 |
| BICF2G630245758 | |
| BICF2P1321188 | |
| BICF2P903863 | |
| BICF2G630340881 | 3 |
| BICF2G630340902 | |
| BICF2G630340909 | |
| BICF2G630340916 | |
| BICF2P1145835 | 33 |
| BICF2S2323286 | |
| BICF2S23628331 | |
| BICF2S23711437 | |
| BICF2P580416 | 20 |
| BICF2P642325 | |
| BICF2S23123519 | |
| BICF2S23217200 | |

B. Linkage disequilibrium blocks (R² > 0.8) found within
the SNPs associated to CHD in the GWAS (Table 12) and
in the candidate gene strategy (Table 2). The SNPs in
linkage disequilibrium are in the same box.

| | |
|---|---|
| BICF2G630106718 | 3 |
| BICF2P424215 | |
| BICF2P889439 | |
| BICF2G630106786 | 3 |
| BICF2G630106787 | |
| BICF2G630107204 | |
| BICF2G630244778 | 33 |
| BICF2G630244789 | |
| TIGRP2P390878_rs9092335 | |
| BICF2G630246506 | 33 |
| BICF2G630246514 | |
| BICF2P1007883 | |
| BICF2G630401283 | 30 |
| BICF2G630401334 | |
| BICF2G630401339 | |
| BICF2G630408521 | 30 |
| BICF2P295156 | |
| BICF2S23021949 | |
| BICF2G630532991 | X |
| BICF2G630532995 | |
| BICF2G630533005 | |
| BICF2G63059021 | 4 |
| BICF2G63059130 | |
| BICF2G63059131 | |
| BICF2P1358015 | 4 |
| BICF2P236590 | |
| BICF2P347050 | |
| BICF2P995251 | 33 |
| BICF2S23515275 | |
| BICF2S23546726 | |
| BICF2G630106615 | 3 |
| BICF2G630106625 | |
| BICF2G630106677 | 3 |
| BICF2G630106693 | |
| BICF2G630245484 | 33 |
| BICF2P839475 | |
| BICF2G630245491 | 33 |
| BICF2P1136726 | |
| BICF2G630533695 | X |
| BICF2G630533696 | |

C. Linkage disequilibrium blocks (R² > 0.8) found within
the SNPs associated to CHD in the GWAS (Table 12) and
in the candidate gene strategy (Table 2). The SNPs in
linkage disequilibrium are in the same box.

| | |
|---|---|
| BICF2G630534587 | X |
| BICF2G630534598 | |
| BICF2G630558118 | 7 |
| BICF2G630558172 | |
| BICF2G63058908 | 4 |
| BICF2G63058969 | |
| BICF2G630704471 | 3 |
| BICF2P664113 | |
| BICF2G630830616 | 9 |
| BICF2G630830621 | |
| BICF2P1009099 | 4 |
| BICF2P235645 | |
| BICF2P1018431 | 4 |
| BICF2P243838 | |
| BICF2P1045684 | 35 |
| BICF2P787863 | |
| BICF2P1090418 | 4 |
| TIGRP2P58893_rs9244440 | |
| BICF2P1097570 | 12 |
| TIGRP2P158471_rs8951942 | |
| BICF2P283225 | 15 |
| TIGRP2P194963_rs8923342 | |
| BICF2P349191 | 8 |
| BICF2P900262 | |
| BICF2P525869 | 3 |
| BICF2P618822 | |
| BICF2P526962 | 10 |
| BICF2P823840 | |
| BICF2P909639 | 35 |
| TIGRP2P410898_rs8604820 | |
| BICF2S22914443 | 14 |
| BICF2S23447436 | |
| BICF2S23357027 | 12 |
| TIGRP2P158316_rs9164582 | |
| TIGRP2P268225_rs8813006 | 19 |
| TIGRP2P268234_rs9104397 | |

Seventeen of the SNPs found to be associated to CHD and OA in the candidate gene strategy (Table 2 and 9) and in the GWAS (Table 12) were located in exonic regions. From the 17 exonic SNPs, 5 were non-synonymous (Table 14A) and 12 were synonymous (Table 14B).

TABLE 14A
Non-synonymous exonic SNPs associated to canine
hip dysplasia and osteoarthritis.

| | SNP code | | Amino acid |
|---|---|---|---|
| SNP number | (CanFam2.0) | Gene | change |
| C32 | | CHST3 | Arg/Gly |
| 15 | BICF2P853899 | CSPG2/VCAN | Lys/Asn |
| 22 | BICF2S23042158 | MIG6/ERRFI1 | Val/Met |
| 128 | BICF2P178723 | NCOR2 | Trp/STOP |
| 177 | BICF2P876960 | TAS1R2 | Met/Thr |

TABLE 14B
Synonymous exonic SNPs associated to canine
hip dysplasia and osteoarthritis.

| SNP | SNP code | | |
|---|---|---|---|
| number | (CanFam2.0) | Gene | Amino acid |
| 19 | BICF2P525802 | CSPG1/AGC1 | Thr/Thr |
| 78 | BICF2G630295186 | MEGF10 | Ileu/Ileu |
| 88 | BICF2P966124 | FBN2 | Asn/Asn |
| 99 | BICF2P968235 | COL1A2 | Val/Val |
| 102 | BICF2G630217408 | MATN3 | Cys/Cys |
| 113 | BICF2S23632685 | FLNB | Ileu/Ileu |
| 126 | | ADAM28 | Val/Val |
| 129 | BICF2P133720 | NCOR2 | Ala/Ala |
| 150 | BICF2P643437 | SULF1 | Leu/Leu |
| 158 | BICF2G630403760 | ADAM10 | Arg/Arg |
| 161 | BICF2P1202421 | ADAMTS5 | Asp/Asp |
| 188 | BICF2G630704471 | Q32K14 | Tyr/Tyr |

We selected the most associated SNPs from both strategies, candidate genes and GWAS, and entered them together with the coat color variable into forward logistic regression modeling process to investigate predictors for CHD and OA. When 2 SNP5 were in linkage disequilibrium (R²<0.8) only the one with the lowest p value for allelic association was included in the multivariate logistic regression analysis. We present herein, as non limiting examples, seven predictive models with a good accuracy for CHD and OA prediction (area under the ROC curve (AUC) over 80%) (FIGS. 8 A, B, C, D, E, F and G). The SNP C38 of the CHST3 gene (SNP 21 of Table 1) remains in five of the models together with other SNP5 outside the CHST3 gene. The SNP C18 of the CHST3 gene (SNP 20 of Table 1) is present in the other two predictive models. The clinical variable coat color is present in four of the models. In the FIGS. 8 A, B, C, D, E, F and G are represented the ROC curves of the seven predictive models and are indicated the clinical and genetic variables which remained in each model and their odds ratio. In Table 15 are depicted the risk genotypes of all the SNPs included in each of the models of FIG. 8.

TABLE 15
Risk genotypes of the SNPs included in the predictive models
of FIGS. 8A, B, C, D, E, F and G.

| Marker | Predictive model | Risk genotype |
|---|---|---|
| C38 (BICF2P419109) | 1, 2, 3, 4, 5 | GG + AG |
| 8 (BICF2S23036087) | 1, 3 | GG + AG |
| 333 (BICF2G630227898) | 1, 3, 6 | AA + AG |
| 211 (BICF2G630339806) | 1, 3, 4, 5, 6 | AA + AG |
| 325 (BICF2S230609) | 1, 3, 6 | AA |
| 348 (BICF2S23217200) | 1, 3 | AA + AC |
| 231 (BICF2S2412468) | 1, 3, 4, 5 | CC + AC |
| 288 (BICF2S2452559) | 1, 3, 6 | GG + AG |
| C32 | 2 | GG + CG |
| 210 (BICF2G630339399) | 2 | GG + AG |
| 276 (BICF2P146712) | 2 | CC + AC |
| 250 (BICF2P1330558) | 2 | AA |
| 275 (BICF2P1420892) | 2 | GG + AG |
| 307 (BICF2S23417189) | 2 | AA |
| 173 (BICF2P1446055) | 4 | GG + AG |
| 255 (BICF2G630558239) | 4, 5, 6 | AA + AG |
| 387 (BICF2G630249309) | 4, 5 | AA |
| 345 (BICF2P580416) | 4, 5 | CC + AC |
| 261 (BICF2P601580) | 4, 5 | GG + CG |
| 229 (BICF2P648799) | 4, 5 | CC + AC |
| 259 (BICF2S23026364) | 4, 5 | AA + AG |
| 301 (BICF2P548082) | 4, 6 | GG + AG |
| C18 (BICF2P772455) | 6 | GG |

To summarize, with the examples presented herein we demonstrate that polymorphisms in the CHST3 gene and predictive models combining polymorphisms in the CHST3 gene with polymorphisms in other genes (Tables 2 and 12) and/or the coat color, allow for discrimination between animals with low and high predisposition or susceptibility for hip dysplasia or osteoarthritis, thus allowing differential treatment management for a given individual to prevent or lessen hip/joint dysplasia and osteoarthritis and selection of individuals with low predisposition for hip/joint dysplasia for breeding.

### Primers and Probes

TABLE 16
Certain preferred probes are shown below, in
pairs, for each of the indicated SNPs.

| | | SEQ |
|---|---|---|
| SNP | Probe sequences (5′-3′) | ID NO |
| BICF2G630227898 | TTAATCTCGCCCTCTTCCC | 97 |
| (SNP No. 333) | GGGAAGAGGGCGAGATTAA | 98 |
| | TTAATCTCGTCCTCTTCCC | 99 |
| | GGGAAGAGGACGAGATTAA | 100 |
| | TAATCTCGCCCTCTTCC | 101 |
| | TAATCTCGTCCTCTTCC | 102 |
| | AATCTCGCCCTCTTCCCTG | 103 |
| | AATCTCGTCCTCTTCCCTG | 104 |
| | GTTTAATCTCGCCCTCTTC | 105 |
| | GTTTAATCTCGTCCTCTTC | 106 |
| BICF2G630339806 | ACACTCTCAGTAACTTGTA | 107 |
| (SNP No. 211) | ACACTCTCAATAACTTGTA | 108 |
| | TACAAGTTATTGAGAGTGT | 109 |
| | TACAAGTTACTGAGAGTGT | 110 |
| | CACTCTCAGTAACTTGT | 111 |
| | CACTCTCAATAACTTGT | 112 |
| BICF2S230609 | TGGGTGAGTCACGACGCAT | 113 |
| (SNP No. 325) | ATGCGTCGTGACTCACCCA | 114 |
| | TGGGTGAGTTACGACGCAT | 115 |
| | ATGCGTCGTAACTCACCCA | 116 |
| | GGGTGAGTCACGACGCA | 117 |
| | GGGTGAGTTACGACGCA | 118 |
| | TGAGTCACGACGCATGAAT | 119 |
| | TGAGTTACGACGCATGAAT | 120 |
| | AATCTGGGTGAGTCACGAC | 121 |
| | AATCTGGGTGAGTTACGAC | 122 |
| | GGTGAGTCACGACGCATGA | 123 |
| | GGTGAGTTACGACGCATGA | 124 |
| | TCTGGGTGAGTCACGACGC | 125 |
| | TCTGGGTGAGTTACGACGC | 126 |
| BICF2S2452559 | CATGTTCACTAAAACACCA | 127 |
| (SNP No. 288) | TGGTGTTTTAGTGAACATG | 128 |
| | CATGTTCACCAAAACACCA | 129 |
| | TGGTGTTTTGGTGAACATG | 130 |
| | ATGTTCACTAAAACACC | 131 |
| | ATGTTCACCAAAACACC | 132 |
| | TTCACTAAAACACCATGGC | 133 |
| | TTCACCAAAACACCATGGC | 134 |
| | GAGTACATGTTCACTAAAA | 135 |
| | GAGTACATGTTCACCAAAA | 136 |
| | TGTTCACTAAAACACCATG | 137 |
| | TGTTCACCAAAACACCATG | 138 |
| | TACATGTTCACTAAAACAC | 139 |
| | TACATGTTCACCAAAACAC | 140 |
| BICF2G630558239 | TTCATGACCCGTTAACTCC | 141 |
| (SNP No. 255) | GGAGTTAACGGGTCATGAA | 142 |
| | TTCATGACCTGTTAACTCC | 143 |
| | GGAGTTAACAGGTCATGAA | 144 |
| | TCATGACCCGTTAACTC | 145 |
| | TCATGACCTGTTAACTC | 146 |
| | ATGACCCGTTAACTCCCCT | 147 |
| | ATGACCTGTTAACTCCCCT | 148 |
| | TTATTCATGACCCGTTAAC | 149 |
| | TTATTCATGACCTGTTAAC | 150 |
| | CATGACCCGTTAACTCCCC | 151 |
| | CATGACCTGTTAACTCCCC | 152 |
| | TATTCATGACCCGTTAACT | 153 |
| | TATTCATGACCTGTTAACT | 154 |
| BICF2P548082 | GTACATTGTATTGTAGATG | 155 |
| (SNP No. 301) | CATCTACAATACAATGTAC | 156 |
| | GTACATTGTGTTGTAGATG | 157 |
| | CATCTACAACACAATGTAC | 158 |
| | TACATTGTATTGTAGAT | 159 |
| | TACATTGTGTTGTAGAT | 160 |
| | ATTGTATTGTAGATGTTTG | 161 |
| | ATTGTGTTGTAGATGTTTG | 162 |
| | GGTAGGTACATTGTATTGT | 163 |
| | GGTAGGTACATTGTGTTGT | 164 |
| | ACATTGTATTGTAGATGTT | 165 |
| | ACATTGTGTTGTAGATGTT | 166 |
| | AGGTACATTGTATTGTAGA | 167 |
| | AGGTACATTGTGTTGTAGA | 168 |
| BICF2P772455 | CCTGACCACTGGTCTCTTC | 169 |
| (SNP No. 20) | GAAGAGACCAGTGGTCAGG | 170 |
| | CCTGACCACCGGTCTCTTC | 171 |
| | GAAGAGACCGGTGGTCAGG | 172 |
| | TCCTGACCACTGGTCTCTTCA | 173 |
| | TCCTGACCACCGGTCTCTTCA | 174 |
| | TGTCCTGACCACTGGTCTC | 175 |
| | GGGGTGTCCTGACCACTGG | 176 |
| | TGTCCTGACCACCGGTCTC | 177 |
| | GGGGTGTCCTGACCACCGG | 178 |
| | GACCACTGGTCTCTTCACA | 179 |
| | GACCACCGGTCTCTTCACA | 180 |
| | CCACTGGTCTCTTCACAGG | 181 |
| | CCACCGGTCTCTTCACAGG | 182 |

The best-performing pair for each SNP is shown in bold.
The nucleotide corresponding to the polymorphic position is shown underlined.

TABLE 17
Certain preferred probes are shown below,
in pairs, for each of the indicated SNPs.

| SNP | Probe sequences (5′-3′) | SEQ ID NO: |
|---|---|---|
| BICF2G630227898 | TAATCTCGCCCTCTTCC | 101 |
| (SNP No. 333) | TAATCTCGTCCTCTTCC | 102 |
| BICF2G630339806 | ACACTCTCAGTAACTTGTA | 107 |
| (SNP No. 211) | ACACTCTCAATAACTTGTA | 108 |
| BICF2S230609 | TCTGGGTGAGTCACGACGC | 125 |
| (SNP No. 325) | TCTGGGTGAGTTACGACGC | 126 |
| BICF2S2452559 | TACATGTTCACTAAAACAC | 139 |
| (SNP No. 288) | TACATGTTCACCAAAACAC | 140 |
| BICF2G630558239 | TATTCATGACCCGTTAACT | 153 |
| (SNP No. 255) | TATTCATGACCTGTTAACT | 154 |
| BICF2P548082 | ACATTGTATTGTAGATGTT | 165 |
| (SNP No. 301) | ACATTGTGTTGTAGATGTT | 166 |
| BICF2P772455 | CCACTGGTCTCTTCACAGG | 181 |
| (SNP No. 20) | CCACCGGTCTCTTCACAGG | 182 |

The probe pairs shown in Table 17 are those shown as the best-performing pair in Table 16 (i.e. the sequences shown in bold in Table 16).
The nucleotide corresponding to the polymorphic position is shown underlined.

TABLE 18
Primers including tags. Certain preferred primer sequences
are shown below, grouped according SNP.

| SNP | Orientation | Sequence (tag sequence shown in bold) 5′-3′ | SEQ ID NO |
|---|---|---|---|
| BICF2P772455 | Forward | AACCTTCAACTACACGGCTCACCTGCCCTTGTAAGTTGGGTGGAA | 183 |
| (SNP No. 20) | Reverse | AAGGAGATTATGTACCGAGGAAGAAGTCTTCAGGTGGGGGACA | 184 |
| BICF2G630227898 | Forward | AACCTTCAACTACACGGCTCACCTGGACTGATCTGTGCCTTCTGC | 185 |
| (SNP No. 333) | Reverse | AAGGAGATTATGTACCGAGGAAGAAGTCCCCGGAATAACGAAAG | 186 |
| | Forward | AACCTTCAACTACACGGCTCACCTGGGACACTACTGTTAGAGCCA | 187 |
| | Reverse | AAGGAGATTATGTACCGAGGAAGAAAGTTGTCGCCATCTTTGAGG | 188 |
| BICF2G630339806 | Forward | AACCTTCAACTACACGGCTCACCTGTGGATAGTTGTGAGGCTTTCC | 189 |
| (SNP No. 211) | Reverse | AAGGAGATTATGTACCGAGGAAGAACATGAACCTTCCAGAAGAGATG | 190 |
| BICF2G630558239 | Forward | AACCTTCAACTACACGGCTCACCTGTCAATTGCCTATGCCTTGTG | 191 |
| (SNP No. 255) | Reverse | AAGGAGATTATGTACCGAGGAAGAACGGAGGTGAAGAACACAACA | 192 |
| BICF2P548082 | Forward | AACCTTCAACTACACGGCTCACCTGTCCAGTTTTTGGTTTTCAGC | 193 |
| (SNP No. 301) | Reverse | AAGGAGATTATGTACCGAGGAAGAACTGAGCACCTCTGTGGATCA | 194 |
| | Reverse | AAGGAGATTATGTACCGAGGAAGAACAAATATGTCTTTAGCAGATAAGC | 195 |
| BICF2S230609 | Forward | AACCTTCAACTACACGGCTCACCTGGGCCTGTGGAGCTGACTG | 196 |
| (SNP No. 325) | Reverse | AAGGAGATTATGTACCGAGGAAGAAACGGCCAATCAACGTCAT | 197 |
| BICF2S2452559 | Forward | AACCTTCAACTACACGGCTCACCTGCAGTTTGTTGGTGCAAGCTC | 198 |
| (SNP No. 288) | Reverse | AAGGAGATTATGTACCGAGGAAGAACTCAGGTGAGGGGGATCTCT | 199 |

The primers comprise a “tag” sequence, that is shown in bold and is one of a limited number of sequences shared by the primers, and a specific sequence that is shown not in bold and which represents the sequence-specific portion of the primer.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, including patent documents, disclosed herein are incorporated by reference in their entirety for all purposes, particularly for the disclosure referenced herein.

### BIBLIOGRAPHY

Caterson B, Mahmoodian F, Sorrell J M, Hardingham T E, Bayliss M T, Carney S L et al. Modulation of native chondroitin sulphate structure in tissue development and in disease. J Cell Sci 1990; 97 (Pt 3):411-417.
Clements D N, Short A D, Barnes A, Kennedy U, Ferguson J F, Butterworth S J et al. A candidate gene study of canine joint diseases. J Hered 2010; 101(1):54-60.
Chase K, Lawler D F, Adler F R, Ostrander E A, Lark K G. Bilaterally asymmetric effects of quantitative trait loci (QTLs): QTLs that affect laxity in the right versus left coxofemoral (hip) joints of the dog (Canis familiaris). Am J Med Genet A 2004; 124A(3):239-247.
Chase K, Lawler D F, Carrier D R, Lark K G (2005) Genetic regulation of osteoarthritis: A QTL regulating cranial and caudal acetabular osteophyte formation in the hip joint of the dog (Canis familiaris). Am J Med Genet A 135: 334-335.
Distl Ottmar (20 May 2008) EP 2123775A1. Analysis for the genetic disposition for hip dysplasia in Canidae.
Distl Ottmar (20 May 2009) EP 2123777A1. Analysis for the genetic disposition for hip dysplasia in Canidae.
Engler J, Hamann H, Distl O. [Estimation of genetic parameters for radiographic signs of hip dysplasia in Labrador Retrievers]. Berl Munch Tierarztl Wochenschr 2008; 121(9-10):359-364.
Fan J B, Oliphant A, Shen R, Kermani B G, Garcia F, Gunderson K L et al. Highly parallel SNP genotyping. Cold Spring Harb Symp Quant Biol 2003; 68:69-78.
Friedenberg et al.
(http://dspace.library.cornell.edu:8080/bitstream/1813/12739/4/Friedenberg-Steve-summary2009.pdf)
Habuchi Osami (31 May 1995) EP0745668(2). DNA coding for sulfotransferase.
Hermanns P, Unger S, Rossi A, Perez-Aytes A, Cortina H, Bonafe L et al. Congenital joint dislocations caused by carbohydrate sulfotransferase 3 deficiency in recessive Larsen syndrome and humero-spinal dysostosis. Am J Hum Genet 2008; 82(6):1368-1374.
Janutta V, Hamann H, Distl O. Complex segregation analysis of canine hip dysplasia in German shepherd dogs. J Hered 2006; 97(1):13-20.
Lee S A, Lee S G, Cho K O, Hyun C. Isolation, characterization and molecular screening of canine SLC26A2 (sulphate transporter) in German Shepherd dogs with hip dysplasia. J Genet 2007; 86(3):285-288.
Maki K, Liinamo A E, Ojala M. Estimates of genetic parameters for hip and elbow dysplasia in Finnish Rottweilers. J Anim Sci 2000; 78(5):1141-1148.
Maki K, Janss L L, Groen A F, Liinamo A E, Ojala M. An indication of major genes affecting hip and elbow dysplasia in four Finnish dog populations. Heredity 2004; 92(5):402-408.
Marschall Y, Distl O. Mapping quantitative trait loci for canine hip dysplasia in German Shepherd dogs. Mamm Genome 2007; 18(12):861-870.
Mateescu R G, Burton-Wurster N I, Tsai K, Phavaphutanon J, Zhang Z, Murphy K E, Lust G, Todhunter R J (2008) Identification of quantitative trait loci for osteoarthritis of hip joints in dogs. Am J Vet Res 69: 1294-1300.
Ratcliffe A, Shurety W, Caterson B. The quantitation of a native chondroitin sulfate epitope in synovial fluid lavages and articular cartilage from canine experimental osteoarthritis and disuse atrophy. Arthritis Rheum 1993; 36(4):543-551.
Sugahara K, Kitagawa H. Recent advances in the study of the biosynthesis and functions of sulfated glycosaminoglycans. Curr Opin Struct Biol 2000; 10(5):518-527.
Thiele H, Sakano M, Kitagawa H, Sugahara K, Rajab A, Hohne W et al. Loss of chondroitin 6-O-sulfotransferase-1 function results in severe human chondrodysplasia with progressive spinal involvement. Proc Natl Acad Sci USA 2004; 101(27):10155-10160.
Tsutsumi K, Shimakawa H, Kitagawa H, Sugahara K. Functional expression and genomic structure of human chondroitin 6-sulfotransferase. FEBS Lett 1998; 441(2):235-241.
Williams Kevin Jon (31 Mar. 1997) U.S. Pat. No. 6,399,358 (B1). Human gene encoding human chondroitin 6-sulfotransferase.
Zhu L, Zhang Z, Feng F, Schweitzer P, Phavaphutanon J, Vernier-Singer M et al. Single nucleotide polymorphisms refine QTL intervals for hip joint laxity in dogs. Anim Genet 2008; 39(2):141-146.

1. A method of predicting risk of joint dysplasia and/or a condition that is secondary to joint dysplasia in a mammalian subject of the order Carnivora, the method comprising:
(a) extracting and/or amplifying nucleic acid from a nucleic acid-containing sample that has been obtained from the subject and analyzing said nucleic acid to determine the genotype of said subject in respect of one or more genetic polymorphisms and/or alterations selected from the group consisting of:
(i) one or more polymorphisms or alterations in the CHST3 gene or a regulatory region thereof; and/or
(ii) one or more single nucleotide polymorphisms in linkage disequilibrium at R²>0.8 with one of: C38 (SEQ ID NO: 96), C18 (SEQ ID NO: 76), C34 (SEQ ID NO: 90), C32 (SEQ ID NO: 88), C36 (SEQ ID NO: 94), C17 (SEQ ID NO: 75), C15 (SEQ ID NO: 73), C6 (SEQ ID NO: 63) and C23 (SEQ ID NO: 80), as set forth in Table 7; and
(b) providing a prediction of the risk of joint dysplasia and/or a condition that is secondary to joint dysplasia in said subject, which prediction is based on said genotype.

2. A method of classifying a mammalian subject of the order Carnivora as predisposed or not predisposed to joint dysplasia and/or a condition that is secondary to joint dysplasia, the method comprising:
(a) extracting and/or amplifying nucleic acid from a nucleic acid-containing sample that has been obtained from the subject and analyzing said nucleic acid to determine the genotype of said subject in respect of one or more genetic polymorphisms and/or alterations selected from the group consisting of:
(i) one or more polymorphisms or alterations in the CHST3 gene or a regulatory region thereof; and/or
(ii) one or more single nucleotide polymorphisms (SNPs) in linkage disequilibrium at R²>0.8 with one of the SNPs selected from: C38 (SEQ ID NO: 96), C18 (SEQ ID NO: 76), C34 (SEQ ID NO: 90), C32 (SEQ ID NO: 88), C36 (SEQ ID NO: 94), C17 (SEQ ID NO: 75), C15 (SEQ ID NO: 73), C6 (SEQ ID NO: 63) and C23 (SEQ ID NO: 80), as set forth in Table 7; and
(b) providing a classification of said subject as predisposed or not predisposed to joint dysplasia and/or a condition that is secondary to joint dysplasia, which classification is based on said genotype.

3. (canceled)

4. The method of claim 1, wherein said sample is selected from the group consisting of: DNA, urine, saliva, blood, serum, faeces, other biological fluids, hair, cells and tissues.

5-6. (canceled)

7. The method of claim 1, wherein analyzing said nucleic acid determines that the subject carries at least one copy of at least one risk allele selected from the group consisting of: G at SNP C38, C at SNP C18, C at SNP C34, G at SNP C32, G at SNP C36, T at SNP C17, T at SNP C15, T at SNP C6 and T at SNP C23, as set forth in Table 7, and wherein said prediction is a prediction of increased risk of joint dysplasia and/or a condition that is secondary to joint dysplasia in said subject.

8-10. (canceled)

11. The method of claim 1, wherein amplifying said nucleic acid comprises amplifying DNA that has been obtained from the subject by performing PCR using one or more oligonucleotide primers of SEQ ID NOs: 12-23 SEQ ID NOs: 24-57 or SEQ ID NOs: 183-184.

12-16. (canceled)

17. The method of claim 1, wherein providing said prediction of the risk of joint dysplasia and/or a condition that is secondary to joint dysplasia in said subject comprises using a probability function.

18. The method of claim 1, wherein analyzing said nucleic acid to determine the genotype of said subject comprises determining the genotype of said subject in respect of two, three, four, five, six, seven, eight, nine or ten or more genetic polymorphisms and/or alterations as defined in claim 1.

19. (canceled)

20. The method of claim 1, further comprising obtaining or determining one or more clinical variables of said subject selected from the group consisting of: coat colour, adult weight, birth weight, gender, age, exercise habits, diet habits, usual type of floor, early spay, mortality before weaning and litter size.

21. The method of claim 1, wherein the method comprises determining for said subject the outcome of each of the variables set forth in FIGS. 8A, 8B, 8C, 80, 8E, 8F and/or 8G.

22. A method for determining the propensity of a mammalian subject of the order Carnivora to respond effectively to treatment with glycosaminoglycans therapy, the method comprising:
(a) extracting and/or amplifying nucleic acid from a nucleic acid-containing sample that has been obtained from the subject and analyzing said nucleic acid to determine whether the subject carries at least one copy of at least one risk allele selected from the group consisting of: G at SNP C38 (SEQ ID NO: 96), C at SNP C18 (SEQ ID NO: 76), C at SNP C34 (SEQ ID NO: 90), G at SNP C32 (SEQ ID NO: 88), G at SNP C36 (SEQ ID NO: 94), T at SNP C17 (SEQ ID NO: 75), T at SNP C15 (SEQ ID NO: 73), T at SNP C6 (SEQ ID NO: 63) and T at SNP C23 (SEQ ID NO: 80), as set forth in Table 7, or an SNP in linkage disequilibrium at R²>0.8 with one of said SNP risk alleles; and
(b) where the subject has been determined to carry at least one copy of at least one of said risk alleles in step (a), selecting the subject as having been determined to have the propensity to respond effectively to said treatment with glycosaminoglycans therapy.

23. A method of selective breeding comprising:
carrying out the method of claim 1 on each of a plurality of mammalian subjects of the order Carnivora, thereby identifying from among said plurality those subjects having increased risk of having or developing joint dysplasia and/or a condition that is secondary to joint dysplasia, and those subjects not having said increased risk; and
selectively breeding from those subjects not having said increased risk.

24. The method of claim 1, wherein said subject is a dog.

25. The method according to claim 24, wherein the subject is a breed of dog selected from the group consisting of: Labrador Retriever, German Shepherd, Golden Retriever, Beagle, Boxer, Bulldogs, Schnauzers, Rottweiler, Pug, Cocker Spaniel, English Springer Spaniel, Dogues Bordeaux, Bullmastiff, Saint Bernard, Gordon Setter, Bernese mountain dog, Saint Bernard and American Staffordshire.

26. The method of claim 1, wherein said joint dysplasia is hip and/or elbow dysplasia.

27. (canceled)

28. The method of claim 1, wherein said condition that is secondary to joint dysplasia is selected from the group consisting of: secondary osteoarthritis, synovitis, muscular atrophy, subcondral bone sclerosis and articular laxitude.

29-48. (canceled)

49. The method of claim 1, wherein analyzing said nucleic acid to determine the genotype of said subject comprises use of one or more probes of SEQ ID NOs: 169-182.

50. The method of claim 49, wherein analyzing said nucleic acid to determine the genotype of said subject comprises use of one or more probe pairs of SEQ ID NOs: 169 & 170, 171 & 172, 173 & 174, 175 & 176, 177 & 178, 179 & 180, and 181& 182.

51. The method of claim 23, wherein said subject is a dog.

52. The method of claim 51, wherein said subject is a breed of dog selected from the group consisting of: Labrador Retriever, German Shepherd, Golden Retriever, Beagle, Boxer, Bulldogs, Schnauzers, Rottweiler, Pug, Cocker Spaniel, English Springer Spaniel, Dogues Bordeaux, Bullmastiff, Saint Bernard, Gordon Setter, Bernese mountain dog, Saint Bernard and American Staffordshire.


SEQUENCE LISTING (199 sequences)

SEQ ID NO: 1 - DNA, 338 bp, Canis familiaris
atatggtgggtgccgttccagacacaagtccctacttcaggggactcggagtctgagaagggaagacagacagaccatcagctggaaagcccaaccatacgtgctatggaaggggtgactggggagcagagagaggctcgagtgacctggaacttggggaggcgatgaaggctgctgttggaggaagctgggacaggtgatttaacccctcggcctcagtttcctcatctataaaatgggatagttgctacctgataggactgtaaggatctgatgaggaaatgcaaatcaagtgcttagcttggcaccggccacagagctgtaagcactcaatgaat

SEQ ID NO: 2 - DNA, 1799 bp, Canis familiaris
atagttgctacctgataggactgtaaggatctgatgaggaaatgcaaatcaagtgcttagcttggcaccggccacagagctgtaagcactcaatgaattttggctacccaaataattccagttggcctttccaaccagaatggaagcttcctgagggtaaggaccaggtctgcctgtttactgctgtatccccagagcctagaccagtggctggcacagagtgggtgctcagaaaggagttgtttagtgagtgtgtggggaaaggcccaggagaattaggaaggggcaggaatattccagttgccctggagctggccctgcaggcctcttccctcctgagcccctctccagtcacccacactcaggacatctcgccacccctctgccccgcacagctgggtgccgccatcagctaccaggctacacatctgcctccttgcgttgcagagcccccacccccctgccttccccaaaggacatgctctgactgtccctctggccacttccattgtatgatgcaggtgcagtcatgggacctaccttcctgccctcctgctcccccatgttgacgtcagctgctttctgtgggaatatccatcttttccagaaggagagatgatcctgggtgccctgcaccaggtcatgagacttctccacacttggagcaaatagtaacccgtggatgaaggtccttacggaaaaagggatagatttttcaattggggggcggggggtaggtgttgtgctggaatctggggctcatgatgcaggacaggggtgtgtgtgatgtgtaggaggaatgcaggaaatgggcaccatcaggccgtgctgtatttgccccagcttttaaaattgtatgcatcttccattgagggtcatttattcctttatccattctgctatgtggtgaccgtgctcaggtgtctcctgtgtgcccagcccagtgctaaaggataatgagggtcctggacaaactccctgcactccacagagggatgatcagtgttgaacaaatcgctacaagtgggctcacattatgacagtctcaggtgctaataggagtctttgcgtggtggtgggcacgggggagccctaacctagccaccgggaggctgctccaggaggctgagacgcgagggccatgtaagggattttggaagggaccgagtgaaggatctagtatatgtattaggttattcgcatcagcactcatagggccatctccatcatcttttatttaggtatttgtagtcttttgcaaatgcagacatttatagtgcaaggaacattcttactgcgcacttgcccttgagttacgtgtgaggtcaaaggataaattgatggaagtgggatgaatgaatcatagggctgattggcatttggaatgatctgatctcacctctcatccactgggagagtgggtctgggtttctggctgcctaatcagggccacaacccttgtaagttgggtggaaaaactcatcttcattcctcatctccagacaggggtgtcctgaccactggtctcttcacaggacaagggtgtcccccacctgaagacgaccagccaggccctgagtgatgctcctgagctgagtgcccaccgccccacccttccccatggagaaaggactcgcttttccccaggactgccgggactttctgcacagcctgagaatgaggagcaaatatgcccttttcctggcttttgtggtggtggtttttgtcttcattgaaaaggaaaataaaatcatatcaaggtgagggatacaagcacaagtcttccttatcagggaggg

SEQ ID NO: 3 - DNA, 5819 bp, Canis familiaris
atatggtgggtgccgttccagacacaagtccctacttcaggggactcggagtctgagaagggaagacagacagaccatcagctggaaagcccaaccatacgtgctatggaaggggtgactggggagcagagagaggctcgagtgacctggaacttggggaggcgatgaaggctgctgttggaggaagctgggacaggtgatttaacccctcggcctcagtttcctcatctataaaatgggatagttgctacctgataggactgtaaggatctgatgaggaaatgcaaatcaagtgcttagcttggcaccggccacagagctgtaagcactcaatgaattttggctacccaaataattccagttggcctttccaaccagaatggaagcttcctgagggtaaggaccaggtctgcctgtttactgctgtatccccagagcctagaccagtggctggcacagagtgggtgctcagaaaggagttgtttagtgagtgtgtggggaaaggcccaggagaattaggaaggggcaggaatattccagttgccctggagctggccctgcaggcctcttccctcctgagcccctctccagtcacccacactcaggacatctcgccacccctctgccccgcacagctgggtgccgccatcagctaccaggctacacatctgcctccttgcgttgcagagcccccacccccctgccttccccaaaggacatgctctgactgtccctctggccacttccattgtatgatgcaggtgcagtcatgggacctaccttcctgccctcctgctcccccatgttgacgtcagctgctttctgtgggaatatccatcttttccagaaggagagatgatcctgggtgccctgcaccaggtcatgagacttctccacacttggagcaaatagtaacccgtggatgaaggtccttacggaaaaagggatagatttttcaattggggggcggggggtaggtgttgtgctggaatctggggctcatgatgcaggacaggggtgtgtgtgatgtgtaggaggaatgcaggaaatgggcaccatcaggccgtgctgtatttgccccagcttttaaaattgtatgcatcttccattgagggtcatttattcctttatccattctgctatgtggtgaccgtgctcaggtgtctcctgtgtgcccagcccagtgctaaaggataatgagggtcctggacaaactccctgcactccacagagggatgatcagtgttgaacaaatcgctacaagtgggctcacattatgacagtctcaggtgctaataggagtctttgcgtggtggtgggcacgggggagccctaacctagccaccgggaggctgctccaggaggctgagacgcgagggccatgtaagggattttggaagggaccgagtgaaggatctagtatatgtattaggttattcgcatcagcactcatagggccatctccatcatcttttatttaggtatttgtagtcttttgcaaatgcagacatttatagtgcaaggaacattcttactgcgcacttgcccttgagttacgtgtgaggtcaaaggataaattgatggaagtgggatgaatgaatcatagggctgattggcatttggaatgatctgatctcacctctcatccactgggagagtgggtctgggtttctggctgcctaatcagggccacaacccttgtaagttgggtggaaaaactcatcttcattcctcatctccagacaggggtgtcctgaccactggtctcttcacaggacaagggtgtcccccacctgaagacgaccagccaggccctgagtgatgctcctgagctgagtgcccaccgccccacccttccccatggagaaaggactcgcttttccccaggactgccgggactttctgcacagcctgagaatgaggagcaaatatgcccttttcctggcttttgtggtggtggtttttgtcttcattgaaaaggaaaataaaatcatatcaaggtgagggatacaagcacaagtcttccttatcagggaggggcggatggggagagaggtagctatagggctcttcgtggacctacaaaggcagtcctgggatccctgggtggcgcagcggtttggcgcctgcctctggcccagggcgcgatcctggagacccaggatcgaatcccacgtcgggctcccggtgcatggagcctgcttctccctctgcctgtgtctctgcctctctctctctctctgtgtgactatcataaataaataaaaaaaaaaaaaaaaaaaaaaaaggcagtccttcaaccagtaatcatcaggcccctcctgtgcccgggcagtgggcctgcacaggtcctcaggactacatagggataacacttgggctctggactcaggcagcctgggttggaatccttattctggctctttctagctgtgacagcttgaacaaattcactcttctgtgtcctaagttctgtaaaatccggttaacgatggggctttccttataaagattaaatcaggacatgtaaaacatttacctcagtgcctggcaggcagttggcatctaatacttgctgaccatgactattattattcatttacttcctggctaggatgatgcctcacattaacaatccataaccagcccgaggccatggttgagggcgagcccatggaaaggcggggtcctggtacacaaaccctgatgggcagggctgccatgtgcagccgtccaggttgtatactgctaaattctggggctgtgcactcacctcgactacactaggaatgccaccccctggaggtggcattccaccttgtgcagtggttctgctggagagcctgctagacacaaaaagatttctagacctgctctcccagcacaacaccttccaagaaagcagccgccagggatggcctgtgtagcaggcactgtgccaccgccccacctgggttcctcagcatggactgttccagttggggcccacagggtcctactgccagcccctgcagctctctgccagagagctttctgtctccccgcacacgggtaagctagggatacctctgggagcagcccttagctaagaccagctggagcttcacacccttgagtgagacaatcctcgggtgtgcccctgtggggttggtttgcccacagggacacctgctcaccctttacagacctctttttctccccacctcacctcctacctctctaccagttctccccgtgatcatgtcccggctaaactcaaatccttgctccaaagtcggcttccagggaaacccagctaagggaggcctggggtgatggccccgggaggatgcttcgggttgcacaagggatggcgcctccggtagctcgctgaccccgatttcccttctttcgcccctagggtgtcagacaagctgaagcagatcccacaacccctggtggacgccaacagcaccgacccggccctgatcttggcggacaacgcgtccctcctgtccctgagcgagctggattcagccttcacccagctgcagggccgcctgcggaacctgagtctgcagctgggcgtagagcccgccggggaggccgaggccgggctggaggccgcggcccgggaaggggggccaccccggcccaccgaggcccggccgcggcgccacgtgctcctcatggccaccacgcgcaccggctcctcgttcgtgggcgagttcttcaaccagcagggcaacatcttctacctcttcgagccgctgtggcacatcgagcgcaccgtgaccttcgagcccggaggcgccaacgcggcgggcgcggccctggtctaccgcgacgtgctcaagcagctgttcctgtgcgacctgtacgtgctggagcacttcatcagcccgctgcccgaggaccacctgacccagttcatgttccgccgcggctccagccgctcgctgtgcgaggagcccgtgtgcacgcccttcgtcaagaaggtcttcgagaagtaccactgcaagaaccgccgctgcgggccgctcaacgtgacgctggccgccgaggcctgccgccgcaaggaccacatggcgctcaaggccgtgcgcatccggcagctggagttcctgcagccgctggccgaggacccgcggctggacctgcgcgtcatccagctggtgcgggacccgcgcgccgtgctggcctcccgcatggtggccttcgccggcaagtacgagggctggaagaagtggctggccgagggccaggcgcgcctgggcgaggaggaggtgcagcggctgcggggcaactgcgagagcatccggctgtcggccgagctgggcctgcggcagcccgcctggctgcgcggccgctacatgctggtgcgctacgaggacgtggcgcgctggccgctgcagaaggcgcgcgagatgtaccgcttcgccggcatcccgctcacggcgcaggtggaggactggatccgcaggaacacgcaggcggcccaggacgccagcggcatctactccacgcagaagaactcctcggagcagttcgagaagtggcgcttcggcatgcccttcaagctggcgcaggtggtgcaggccgcctgcgggcccgccatgcgcctcttcggctaccggctggcgcgggacgccgccgccctggccaaccgctccgtcagcctgctggaggagcggggaaccttctgggtcacgtaggccgccctcctcgtcaaaggcccacctgccttgctcgcgccccggccgcccctgagacggtggctcccgcagagagggtggggggcgcccggccggcggcctctcccgcgggccaggccgtccccccgccgcagcagtaggcgccccctccgccagctagctctcccaccgcagccttggggctggggtctccccgagagcgagccgctgcctgtccggcgaggaccgccagacccgggcctaaggggccgcggcctcccgggcaggccctcgcaggcccacacgggcccagaactgttgacaagcctttttctctctgtctctgtctctgtctctgtctctgtctctctctcacacacgcacatacacaaaacaggcataaatgcctgcggaccctgtgggccagagtccgaggcttaaccagaagggactgtgggcaccgaccggtcacaggcttcctgctgtactcagtttctggatttctacacaatccagtgtgccatctgcacggatccctggggagggaccggaatggcccagaacgtccgaggatggcagttgaggcttttcaaccaagagtgattcagtattttcataaatgaaatagccatgctggttactgatgaaatcagccctgcacagagctacgaaacacacctacatgggtaaagacagtgcctctacgtacaccacacacacacacacacacacacacacacacacacacacaaaaccgtagaaacgcaaatccacatatacacatccacagtcttccttgttgctttaggcccacagggtttaacaatcacacacccaaagaggaggctttaattacatttttagaatcatttggggatggggaaggaaacatggtcccaggccatgaccccattccgaccagatttgggactgaacggtagatacaaaacggccgagtctgtggcccccgcctgccttggtttctcctggctacctcctccggcccattcacgtcctccctgctggcaggcaggatgttttgaaatggtgggccaactctcacgtgactgcagcccaaatgccccgtcccagctgggctggctccctgagagcctgtccatgtttgaaggctgcctcgggcctgac

SEQ ID NO: 4 - DNA, 5776 bp, Canis familiaris
cgtggatgaaggtccttacggaaaaagggatagatttttcaattggggggcggggggtaggtgttgtgctggaatctggggctcatgatgcaggacaggggtgtgtgtgatgtgtaggaggaatgcaggaaatgggcaccatcaggccgtgctgtatttgccccagcttttaaaattgtatgcatcttccattgagggtcatttattcctttatccattctgctatgtggtgaccgtgctcaggtgtctcctgtgtgcccagcccagtgctaaaggataatgagggtcctggacaaactccctgcactccacagagggatgatcagtgttgaacaaatcgctacaagtgggctcacattatgacagtctcaggtgctaataggagtctttgcgtggtggtgggcacgggggagccctaacctagccaccgggaggctgctccaggaggctgagacgcgagggccatgtaagggattttggaagggaccgagtgaaggatctagtatatgtattaggttattcgcatcagcactcatagggccatctccatcatcttttatttaggtatttgtagtcttttgcaaatgcagacatttatagtgcaaggaacattcttactgcgcacttgcccttgagttacgtgtgaggtcaaaggataaattgatggaagtgggatgaatgaatcatagggctgattggcatttggaatgatctgatctcacctctcatccactgggagagtgggtctgggtttctggctgcctaatcagggccacaacccttgtaagttgggtggaaaaactcatcttcattcctcatctccagacaggggtgtcctgaccactggtctcttcacaggacaagggtgtcccccacctgaagacgaccagccaggccctgagtgatgctcctgagctgagtgcccaccgccccacccttccccatggagaaaggactcgcttttccccaggactgccgggactttctgcacagcctgagaatgaggagcaaatatgcccttttcctggcttttgtggtggtggtttttgtcttcattgaaaaggaaaataaaatcatatcaaggtgagggatacaagcacaagtcttccttatcagggaggggcggatggggagagaggtagctatagggctcttcgtggacctacaaaggcagtcctgggatccctgggtggcgcagcggtttggcgcctgcctctggcccagggcgcgatcctggagacccaggatcgaatcccacgtcgggctcccggtgcatggagcctgcttctccctctgcctgtgtctctgcctctctctctctctctgtgtgactatcataaataaataaaaaaaaaaaaaaaaaaaaaaaaggcagtccttcaaccagtaatcatcaggcccctcctgtgcccgggcagtgggcctgcacaggtcctcaggactacatagggataacacttgggctctggactcaggcagcctgggttggaatccttattctggctctttctagctgtgacagcttgaacaaattcactcttctgtgtcctaagttctgtaaaatccggttaacgatggggctttccttataaagattaaatcaggacatgtaaaacatttacctcagtgcctggcaggcagttggcatctaatacttgctgaccatgactattattattcatttacttcctggctaggatgatgcctcacattaacaatccataaccagcccgaggccatggttgagggcgagcccatggaaaggcggggtcctggtacacaaaccctgatgggcagggctgccatgtgcagccgtccaggttgtatactgctaaattctggggctgtgcactcacctcgactacactaggaatgccaccccctggaggtggcattccaccttgtgcagtggttctgctggagagcctgctagacacaaaaagatttctagacctgctctcccagcacaacaccttccaagaaagcagccgccagggatggcctgtgtagcaggcactgtgccaccgccccacctgggttcctcagcatggactgttccagttggggcccacagggtcctactgccagcccctgcagctctctgccagagagctttctgtctccccgcacacgggtaagctagggatacctctgggagcagcccttagctaagaccagctggagcttcacacccttgagtgagacaatcctcgggtgtgcccctgtggggttggtttgcccacagggacacctgctcaccctttacagacctctttttctccccacctcacctcctacctctctaccagttctccccgtgatcatgtcccggctaaactcaaatccttgctccaaagtcggcttccagggaaacccagctaagggaggcctggggtgatggccccgggaggatgcttcgggttgcacaagggatggcgcctccggtagctcgctgaccccgatttcccttctttcgcccctagggtgtcagacaagctgaagcagatcccacaacccctggtggacgccaacagcaccgacccggccctgatcttggcggacaacgcgtccctcctgtccctgagcgagctggattcagccttcacccagctgcagggccgcctgcggaacctgagtctgcagctgggcgtagagcccgccggggaggccgaggccgggctggaggccgcggcccgggaaggggggccaccccggcccaccgaggcccggccgcggcgccacgtgctcctcatggccaccacgcgcaccggctcctcgttcgtgggcgagttcttcaaccagcagggcaacatcttctacctcttcgagccgctgtggcacatcgagcgcaccgtgaccttcgagcccggaggcgccaacgcggcgggcgcggccctggtctaccgcgacgtgctcaagcagctgttcctgtgcgacctgtacgtgctggagcacttcatcagcccgctgcccgaggaccacctgacccagttcatgttccgccgcggctccagccgctcgctgtgcgaggagcccgtgtgcacgcccttcgtcaagaaggtcttcgagaagtaccactgcaagaaccgccgctgcgggccgctcaacgtgacgctggccgccgaggcctgccgccgcaaggaccacatggcgctcaaggccgtgcgcatccggcagctggagttcctgcagccgctggccgaggacccgcggctggacctgcgcgtcatccagctggtgcgggacccgcgcgccgtgctggcctcccgcatggtggccttcgccggcaagtacgagggctggaagaagtggctggccgagggccaggcgcgcctgggcgaggaggaggtgcagcggctgcggggcaactgcgagagcatccggctgtcggccgagctgggcctgcggcagcccgcctggctgcgcggccgctacatgctggtgcgctacgaggacgtggcgcgctggccgctgcagaaggcgcgcgagatgtaccgcttcgccggcatcccgctcacggcgcaggtggaggactggatccgcaggaacacgcaggcggcccaggacgccagcggcatctactccacgcagaagaactcctcggagcagttcgagaagtggcgcttcggcatgcccttcaagctggcgcaggtggtgcaggccgcctgcgggcccgccatgcgcctcttcggctaccggctggcgcgggacgccgccgccctggccaaccgctccgtcagcctgctggaggagcggggaaccttctgggtcacgtaggccgccctcctcgtcaaaggcccacctgccttgctcgcgccccggccgcccctgagacggtggctcccgcagagagggtggggggcgcccggccggcggcctctcccgcgggccaggccgtccccccgccgcagcagtaggcgccccctccgccagctagctctcccaccgcagccttggggctggggtctccccgagagcgagccgctgcctgtccggcgaggaccgccagacccgggcctaaggggccgcggcctcccgggcaggccctcgcaggcccacacgggcccagaactgttgacaagcctttttctctctgtctctgtctctgtctctgtctctgtctctctctcacacacgcacatacacaaaacaggcataaatgcctgcggaccctgtgggccagagtccgaggcttaaccagaagggactgtgggcaccgaccggtcacaggcttcctgctgtactcagtttctggatttctacacaatccagtgtgccatctgcacggatccctggggagggaccggaatggcccagaacgtccgaggatggcagttgaggcttttcaaccaagagtgattcagtattttcataaatgaaatagccatgctggttactgatgaaatcagccctgcacagagctacgaaacacacctacatgggtaaagacagtgcctctacgtacaccacacacacacacacacacacacacacacacacacacacaaaaccgtagaaacgcaaatccacatatacacatccacagtcttccttgttgctttaggcccacagggtttaacaatcacacacccaaagaggaggctttaattacatttttagaatcatttggggatggggaaggaaacatggtcccaggccatgaccccattccgaccagatttgggactgaacggtagatacaaaacggccgagtctgtggcccccgcctgccttggtttctcctggctacctcctccggcccattcacgtcctccctgctggcaggcaggatgttttgaaatggtgggccaactctcacgtgactgcagcccaaatgccccgtcccagctgggctggctccctgagagcctgtccatgtttgaaggctgcctcgggcctgactggagccaaaaagcagctcctgagtcctgccatttccagtcaggccttgtgtggggccgggcctaggagagaatgcagagtgctcttgggggagaagtcacatcagggtaccccctcccctgagaagacgggggacagaggtcagcccctggttggtcaccatggttacatggttagtgtctggaattccagctttcagggctctgggaagagtgttgctcaacttgggataggctgggtgttttatttggactttttaaaaaaaaaaagttatttatagtttggtatcttgttttgtggctatgtttctgattttgtttctgcaggagtgatgcttggaagtgatctgggcttgagggggctgggaggaccagcagggagcactctggctcctgctgaagggagaggaggaggaggaagagctttccccagaaggaacagagcctaggcggccacctggcatcctgcagtgccccacgggcttttctgtttgaaccccatgtggaatgaatctcagaaaccaccagggttctttgctgtctatccagaagcaacataaatcagctctcaccctctccccgagatgaggcctgatttgaacatctcttccaggtccaaatgcatctcctggggggtcaggggcctatggccaaacagcaaatgtccccaaactggggagcagaagtgagtccacctcagaacttgaagagctgcccaagttcacaagggctgtttctcatttcctggacaacttctatgtcagatctgtcctgttgtcaaacccagagggctgagtttcagtttgagtaacatgggcacaagggaaatagaggatctgagtccagctggtgctgccatggggagtgtgtcctgg

SEQ ID NO: 5 - DNA, 1920 bp, Canis familiaris
  misc_feature (159)..(798): n is a, c, g or t
gaggaagctgggacaggtgatttaacccctcggcctcagtttcctcatctataaaatgggatagttgctacctgataggactgtaaggatctgatgaggaaatgcaaatcaagtgcttagcttggcaccggccacagagctgtaagcactcaatgaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgtggatgaaggtccttacggaaaaagggatagatttttcaattggggggcggggggtaggtgttgtgctggaatctggggctcatgatgcaggacaggggtgtgtgtgatgtgtaggaggaatgcaggaaatgggcaccatcaggccgtgctgtatttgccccagcttttaaaattgtatgcatcttccattgagggtcatttattcctttatccattctgctatgtggtgaccgtgctcaggtgtctcctgtgtgcccagcccagtgctaaaggataatgagggtcctggacaaactccctgcactccacagagggatgatcagtgttgaacaaatcgctacaagtgggctcacattatgacagtctcaggtgctaataggagtctttgcgtggtggtgggcacgggggagccctaacctagccaccgggaggctgctccaggaggctgagacgcgagggccatgtaagggattttggaagggaccgagtgaaggatctagtatatgtattaggttattcgcatcagcactcatagggccatctccatcatcttttatttaggtatttgtagtcttttgcaaatgcagacatttatagtgcaaggaacattcttactgcgcacttgcccttgagttacgtgtgaggtcaaaggataaattgatggaagtgggatgaatgaatcatagggctgattggcatttggaatgatctgatctcacctctcatccactgggagagtgggtctgggtttctggctgcctaatcagggccacaacccttgtaagttgggtggaaaaactcatcttcattcctcatctccagacaggggtgtcctgaccactggtctcttcacaggacaagggtgtcccccacctgaagacgaccagccaggccctgagtgatgctcctgagctgagtgcccaccgccccacccttccccatggagaaaggactcgcttttccccaggactgccgggactttctgcacagcctgagaatgaggagcaaatatgcccttttcctggcttttgtggtggtggtttttgtcttcattgaaaaggaaaataaaatcatatcaaggtgagggatacaagcacaagtcttccttatcagggaggg

SEQ ID NO: 6 - DNA, 1447 bp, Homo sapiens
taaacacaaatccttgcctcaacgtctgcttttggggaaacccaactgagacaggttttggtgatctgcttggaggactgcttagggttgcccaggagacggggtccccagtcagccactgaccctgatcttctttgtcctcacagggtctcagacaagctgaagcagattccccaagctctagcagatgccaacagcaccgacccagccctgatcttagctgagaacgcatctctcttgtccctgagcgagctcgattcagccttctcccagcttcagagccgtctccgcaacctcagcttgcagctgggcgtggagccagccatggaggccgcaggggaggaagaggaagagcagagaaaggaggaggagccgcccagaccggccgtggcggggccccggcgccacgtgctgctcatggccaccacgcgcaccggctcctcgttcgtgggcgagttcttcaaccagcagggcaacatcttctacctcttcgagccgctgtggcacatcgagcgcacagtgtccttcgagccggggggcgccaacgccgcgggctcggccctggtgtaccgcgacgtgctcaagcagctcttcctgtgcgacctgtacgtgctggagcacttcatcacgccgctgcccgaggaccacctgactcagttcatgttccgccggggctccagccgctccctgtgcgaggaccccgtctgtacgcccttcgtcaagaaggtcttcgagaagtaccactgcaagaaccgccgctgcggccccctcaacgtgacgctggccgcagaggcctgccgccgcaaggagcacatggccctcaaggcggtgcgcatccggcagctggagttcctgcagccgctggccgaggacccccgcctggacctgcgcgtcatccagctggtgcgcgacccccgggccgtgctggcctcgcgcatggtggccttcgccggcaagtataagacctggaagaagtggctggacgacgagggccaggacggcctgagggaagaggaggtgcagcggctgcggggcaactgcgagagcatccgcctgtccgcggagctggggctgcggcagcccgcctggctgcggggccgctacatgctggtgcgctacgaggacgtggcacgcgggccgctgcagaaggcccgcgagatgtaccgcttcgccggcatccccctgaccccgcaggtggaagactggatccaaaagaacacgcaggcggcccacgacggcagcggcatctactccacgcagaagaactcctcggagcagttcgagaagtggcgcttcagcatgcccttcaagctggcccaggtggtgcaggccgcctgcggccctgccatgcgcctcttcggctacaaactggcgcgggacgccgccgccctcaccaaccgctcagtcagcctgctggaggagaggggcaccttctgggtcacgtagg

SEQ ID NO: 7 - DNA, 1445 bp, Canis familiaris
taaactcaaatccttgctccaaagtcggcttccagggaaacccagctaagggaggcctggggtgatggccccgggaggatgcttcgggttgcacaagggatggcgcctccggtagctcgctgaccccgatttcccttctttcgcccctagggtgtcagacaagctgaagcagatcccacaacccctggtggacgccaacagcaccgacccggccctgatcttggcggacaacgcgtccctcctgtccctgagcgagctggattcagccttcacccagctgcagggccgcctgcggaacctgagtctgcagctgggcgtagagcccgccggggaggccgaggccgggctggaggccgcggcccgggaaggggggccaccccggcccaccgaggcccggccgcggcgccacgtgctcctcatggccaccacgcgcaccggctcctcgttcgtgggcgagttcttcaaccagcagggcaacatcttctacctcttcgagccgctgtggcacatcgagcgcaccgtgaccttcgagcccggaggcgccaacgcggcgggcgcggccctggtctaccgcgacgtgctcaagcagctgttcctgtgcgacctgtacgtgctggagcacttcatcagcccgctgcccgaggaccacctgacccagttcatgttccgccgcggctccagccgctcgctgtgcgaggagcccgtgtgcacgcccttcgtcaagaaggtcttcgagaagtaccactgcaagaaccgccgctgcgggccgctcaacgtgacgctggccgccgaggcctgccgccgcaaggaccacatggcgctcaaggccgtgcgcatccggcagctggagttcctgcagccgctggccgaggacccgcggctggacctgcgcgtcatccagctggtgcgggacccgcgcgccgtgctggcctcccgcatggtggccttcgccggcaagtacgagggctggaagaagtggctggccgagggccaggcgcgcctgggcgaggaggaggtgcagcggctgcggggcaactgcgagagcatccggctgtcggccgagctgggcctgcggcagcccgcctggctgcgcggccgctacatgctggtgcgctacgaggacgtggcgcgctggccgctgcagaaggcgcgcgagatgtaccgcttcgccggcatcccgctcacggcgcaggtggaggactggatccgcaggaacacgcaggcggcccaggacgccagcggcatctactccacgcagaagaactcctcggagcagttcgagaagtggcgcttcggcatgcccttcaagctggcgcaggtggtgcaggccgcctgcgggcccgccatgcgcctcttcggctaccggctggcgcgggacgccgccgccctggccaaccgctccgtcagcctgctggaggagcggggaaccttctgggtcacgtagg

SEQ ID NO: 8 - DNA, 472 bp, Homo sapiens
ggagagtgggtctggggtcctggctgctaaccagggccgagatcctcaaaagtctggtggagagacatcctctgcattcctcgtgtacagacaagggtgttctgaccacctgtctctccgcaggacaagggtgtcccccacctgaagacggcaagctgggtcctgagtgatgcccctcagctgagtgtccaaggctggcccgaggagcccccacggccccacctttccccatggagaaaggactcactttgccccaggactgccgggactttgtgcacagcctgaagatgagaagcaaatacgcccttttcttggtttttgtggtgatagtttttgtcttcatcgaaaaggaaaataaaatcatatcaaggtgagggttgcaagcccaagtcttcatcttcctttcaaggtggggtgggtggggacagggaggtaactgcagggctctctgtggacctgcaaatgcattcct

SEQ ID NO: 9 - DNA, 443 bp, Canis familiaris
ggagagtgggtctgggtttctggctgcctaatcagggccacaacccttgtaagttgggtggaaaaactcatcttcattcctcatctccagacaggggtgtcctgaccactggtctcttcacaggacaagggtgtcccccacctgaagacgaccagccaggccctgagtgatgctcctgagctgagtgcccaccgccccacccttccccatggagaaaggactcgcttttccccaggactgccgggactttctgcacagcctgagaatgaggagcaaatatgcccttttcctggcttttgtggtggtggtttttgtcttcattgaaaaggaaaataaaatcatatcaaggtgagggatacaagcacaagtcttccttatcagggaggggcggatggggagagaggtagctatagggctcttcgtggacctacaaaggcagtcct

SEQ ID NO: 10 - DNA, 348 bp, Homo sapiens
tttggactgaacagtggattcagaactgcagcgttcaaagcccctgcccgccttagattctcatggctacctcctctgacccatccacatccttgcggctggcaggcaggatgttttgaaatggtgcagccaaaggccccgtctagcttggctggctcccgaacatgtccatatttgaaggctgcctcaggcctggcaggcagaagcagctgtggtccctggaggcaaaaggcagctcacggggccttccatttccagccaggcctcatctggggtagggccaagaggaaagtacagagtgcccgtgggggagaagtcccaccaggatgccccccctcccctgagaag

SEQ ID NO: 11 - DNA, 350 bp, Canis familiaris
tttgggactgaacggtagatacaaaacggccgagtctgtggcccccgcctgccttggtttctcctggctacctcctccggcccattcacgtcctccctgctggcaggcaggatgttttgaaatggtgggccaactctcacgtgactgcagcccaaatgccccgtcccagctgggctggctccctgagagcctgtccatgtttgaaggctgcctcgggcctgactggagccaaaaagcagctcctgagtcctgccatttccagtcaggccttgtgtggggccgggcctaggagagaatgcagagtgctcttgggggagaagtcacatcagggtaccccctcccctgagaag

SEQ ID NO: 12 - DNA, 20 bp, Artificial sequence
  Synthetic sequence Primer
agcagagagaggctcgagtg

SEQ ID NO: 13 - DNA, 20 bp, Artificial sequence
  Synthetic sequence Primer
gccaatcagccctatgattc

SEQ ID NO: 14 - DNA, 22 bp, Artificial sequence
  Synthetic sequence Primer
gtgcccagcccagtgctaaagg

SEQ ID NO: 15 - DNA, 20 bp, Artificial sequence
  Synthetic sequence Primer
ccagagcccaagtgttatcc

SEQ ID NO: 16 - DNA, 20 bp, Artificial sequence
  Synthetic sequence Primer
gcttttgtggtggtggtttt

SEQ ID NO: 17 - DNA, 20 bp, Artificial sequence
  Synthetic sequence Primer
cccatcagggtttgtgtacc

SEQ ID NO: 18 - DNA, 19 bp, Artificial sequence
  Synthetic sequence Primer
aacgatggggctttcctta

SEQ ID NO: 19 - DNA, 20 bp, Artificial sequence
  Synthetic sequence Primer
ccagctgcagactcaggttc

SEQ ID NO: 20 - DNA, 20 bp, Artificial sequence
  Synthetic sequence Primer
tgtcccggctaaactcaaat

SEQ ID NO: 21 - DNA, 20 bp, Artificial sequence
  Synthetic sequence Primer
cccacagtcccttctggtta

SEQ ID NO: 22 - DNA, 20 bp, Artificial sequence
  Synthetic sequence Primer
ggcccagaactgttgacaag

SEQ ID NO: 23 - DNA, 20 bp, Artificial sequence
  Synthetic sequence Primer
acaaggcctgactggaaatg

SEQ ID NO: 24 - DNA, 22 bp, Artificial sequence
  Synthetic sequence Primer
gtgcccagcccagtgctaaagg

SEQ ID NO: 25 - DNA, 20 bp, Artificial sequence
  Synthetic sequence Primer
tggcatttggaatgatctga

SEQ ID NO: 26 - DNA, 20 bp, Artificial sequence
  Synthetic sequence Primer
gcttttgtggtggtggtttt

SEQ ID NO: 27 - DNA, 20 bp, Artificial sequence
  Synthetic sequence Primer
tgtcccggctaaactcaaat

SEQ ID NO: 28 - DNA, 20 bp, Artificial sequence
  Synthetic sequence Primer
gcgagttcttcaaccagcag

SEQ ID NO: 29 - DNA, 20 bp, Artificial sequence
  Synthetic sequence Primer
caagtacgagggctggaaga

SEQ ID NO: 30 - DNA, 20 bp, Artificial sequence
  Synthetic sequence Primer
ggaaccttctgggtcacgta

SEQ ID NO: 31 - DNA, 20 bp, Artificial sequence
  Synthetic sequence Primer
cagagtccgaggcttaacca

SEQ ID NO: 32 - DNA, 20 bp, Artificial sequence
  Synthetic sequence Primer
agcagagagaggctcgagtg

SEQ ID NO: 33 - DNA, 19 bp, Artificial sequence
  Synthetic sequence Primer
gtgcttagcttggcaccgg

SEQ ID NO: 34 - DNA, 20 bp, Artificial sequence
  Synthetic sequence Primer
cgtggatgaaggtccttacg

SEQ ID NO: 35 - DNA, 20 bp, Artificial sequence
  Synthetic sequence Primer
atagggctcttcgtggacct

SEQ ID NO: 36 - DNA, 19 bp, Artificial sequence
  Synthetic sequence Primer
aacgatggggctttcctta

SEQ ID NO: 37 - DNA, 20 bp, Artificial sequence
  Synthetic sequence Primer
ggttcctcagcatggactgt

SEQ ID NO: 38 - DNA, 20 bp, Artificial sequence
  Synthetic sequence Primer
ggcccagaactgttgacaag

SEQ ID NO: 39 - DNA, 19 bp, Artificial sequence
  Synthetic sequence Primer
accagatttgggactgaac

SEQ ID NO: 40 - DNA, 20 bp, Artificial sequence
  Synthetic sequence Primer
gcagttgaggcttttcaacc

SEQ ID NO: 41 - DNA, 21 bp, Artificial sequence
  Synthetic sequence Primer
tctcacacacgcacatacaca

SEQ ID NO: 42 - DNA, 20 bp, Artificial sequence
  Synthetic sequence Primer
cccacagtcccttctggtta

SEQ ID NO: 43 - DNA, 20 bp, Artificial sequence
  Synthetic sequence Primer
cctacgtgacccagaaggtt

SEQ ID NO: 44 - DNA, 20 bp, Artificial sequence
  Synthetic sequence Primer
gtagcgcaccagcatgtagc

SEQ ID NO: 45 - DNA, 20 bp, Artificial sequence
  Synthetic sequence Primer
atgaactgggtcaggtggtc

SEQ ID NO: 46 - DNA, 20 bp, Artificial sequence
  Synthetic sequence Primer
ccagctgcagactcaggttc

SEQ ID NO: 47 - DNA, 20 bp, Artificial sequence
  Synthetic sequence Primer
cccatcagggtttgtgtacc

SEQ ID NO: 48 - DNA, 20 bp, Artificial sequence
  Synthetic sequence Primer
ccagagcccaagtgttatcc

SEQ ID NO: 49 - DNA, 20 bp, Artificial sequence
  Synthetic sequence Primer
agccaggaaaagggcatatt

SEQ ID NO: 50 - DNA, 19 bp, Artificial sequence
  Synthetic sequence Primer
attcgatcctgggtctcca

SEQ ID NO: 51 - DNA, 20 bp, Artificial sequence
  Synthetic sequence Primer
gccaatcagccctatgattc

SEQ ID NO: 52 - DNA, 18 bp, Artificial sequence
  Synthetic sequence Primer
ctcagcctcctggagcag

SEQ ID NO: 53 - DNA, 20 bp, Artificial sequence
  Synthetic sequence Primer
atcacacacacccctgtcct

SEQ ID NO: 54 - DNA, 21 bp, Artificial sequence
  Synthetic sequence Primer
tcccagaggtatccctagctt

SEQ ID NO: 55 - DNA, 20 bp, Artificial sequence
  Synthetic sequence Primer
acaaggcctgactggaaatg

SEQ ID NO: 56 - DNA, 20 bp, Artificial sequence
  Synthetic sequence Primer
aaagcctcctctttgggtgt

SEQ ID NO: 57 - DNA, 22 bp, Artificial sequence
  Synthetic sequence Primer
tggtgtacgtagaggcactgtc

SEQ ID NO: 58 - DNA, 21 bp, Canis familiaris
aaaatgggatmgttgctacct

SEQ ID NO: 59 - DNA, 21 bp, Canis familiaris
gttgctacctrataggactgt

SEQ ID NO: 60 - DNA, 21 bp, Canis familiaris
agcactcaatraattttggct

SEQ ID NO: 61 - DNA, 21 bp, Canis familiaris
ttaggaagggrcaggaatatt

SEQ ID NO: 62 - DNA, 21 bp, Canis familiaris
cccctctccartcacccacac

SEQ ID NO: 63 - DNA, 21 bp, Canis familiaris
ccctctgcccygcacagctgg

SEQ ID NO: 64 - DNA, 21 bp, Canis familiaris
agctgggtgcygccatcagct

SEQ ID NO: 65 - DNA, 21 bp, Canis familiaris
gagcccccacycccctgcctt

SEQ ID NO: 66 - DNA, 21 bp, Canis familiaris
cttccattgtrtgatgcaggt

SEQ ID NO: 67 - DNA, 21 bp, Canis familiaris
ggcggggggtrggtgttgtgc

SEQ ID NO: 68 - DNA, 20 bp, Canis familiaris
gtgtgtgatgtgtaggagga

SEQ ID NO: 69 - DNA, 20 bp, Canis familiaris
gtgtgtgatggtgaggagga

SEQ ID NO: 70 - DNA, 21 bp, Canis familiaris
aaactccctgmactccacaga

SEQ ID NO: 71 - DNA, 21 bp, Canis familiaris
gtgggctcacrttatgacagt

SEQ ID NO: 72 - DNA, 21 bp, Canis familiaris
ggaagggaccragtgaaggat

SEQ ID NO: 73 - DNA, 21 bp, Canis familiaris
tctccatcatyttttatttag

SEQ ID NO: 74 - DNA, 21 bp, Canis familiaris
tcttactgcgyacttgccctt

SEQ ID NO: 75 - DNA, 21 bp, Canis familiaris
ctcacctctcwtccactggga

SEQ ID NO: 76 - DNA, 22 bp, Canis familiaris
gtcctgaccacyggtctcttca

SEQ ID NO: 77 - DNA, 21 bp, Canis familiaris
cagggaggggyggatggggag

SEQ ID NO: 78 - DNA, 21 bp, Canis familiaris
ataaaaaaaawaaaaaaaaaa

SEQ ID NO: 79 - DNA, 21 bp, Canis familiaris
agtgggcctgyacaggtcctc

SEQ ID NO: 80 - DNA, 21 bp, Canis familiaris
tgcacaggtcytcaggactac

SEQ ID NO: 81 - DNA, 21 bp, Canis familiaris
ccaccccctgraggtggcatt

SEQ ID NO: 82 - DNA, 21 bp, Canis familiaris
gactgttccasttggggccca

SEQ ID NO: 83 - DNA, 21 bp, Canis familiaris
gggagcagccyttagctaaga

SEQ ID NO: 84 - DNA, 21 bp, Canis familiaris
agacaatcctygggtgtgccc

SEQ ID NO: 85 - DNA, 21 bp, Canis familiaris
agacaatcctcrggtgtgccc

SEQ ID NO: 86 - DNA, 21 bp, Canis familiaris
cgggaggatgyttcgggttgc

SEQ ID NO: 87 - DNA, 21 bp, Canis familiaris
cagacaagctraagcagatcc

SEQ ID NO: 88 - DNA, 21 bp, Canis familiaris
ggccgcggccsgggaaggggg

SEQ ID NO: 89 - DNA, 21 bp, Canis familiaris
gcgccaacgcsgcgggcgcgg

SEQ ID NO: 90 - DNA, 21 bp, Canis familiaris
aggaccacctsacccagttca

SEQ ID NO: 91 - DNA, 24 bp, Canis familiaris
tctctgtctctgtctctgtctctg

SEQ ID NO: 92 - DNA, 30 bp, Canis familiaris
tctctgtctctgtctctgtctctgtctctg

SEQ ID NO: 93 - DNA, 36 bp, Canis familiaris
tctctgtctctgtctctgtctctgtctctgtctctg

SEQ ID NO: 94 - DNA, 21 bp, Canis familiaris
acagagctacraaacacacct

SEQ ID NO: 95 - DNA, 20 bp, Canis familiaris
agatacaaaayggccgagtc

SEQ ID NO: 96 - DNA, 21 bp, Canis familiaris
acgtgactgcrgcccaaatgc

SEQ ID NO: 97 - DNA, 19 bp, Artificial sequence
  Synthetic sequence Probe
ttaatctcgccctcttccc

SEQ ID NO: 98 - DNA, 19 bp, Artificial sequence
  Synthetic sequence Probe
gggaagagggcgagattaa

SEQ ID NO: 99 - DNA, 19 bp, Artificial sequence
  Synthetic sequence Probe
ttaatctcgtcctcttccc

SEQ ID NO: 100 - DNA, 19 bp, Artificial sequence
  Synthetic sequence Probe
gggaagaggacgagattaa

SEQ ID NO: 101 - DNA, 17 bp, Artificial sequence
  Synthetic sequence Probe
taatctcgccctcttcc

SEQ ID NO: 102 - DNA, 17 bp, Artificial sequence
  Synthetic sequence Probe
taatctcgtcctcttcc

SEQ ID NO: 103 - DNA, 19 bp, Artificial sequence
  Synthetic sequence Probe
aatctcgccctcttccctg

SEQ ID NO: 104 - DNA, 19 bp, Artificial sequence
  Synthetic sequence Probe
aatctcgtcctcttccctg

SEQ ID NO: 105 - DNA, 19 bp, Artificial sequence
  Synthetic sequence Probe
gtttaatctcgccctcttc

SEQ ID NO: 106 - DNA, 19 bp, Artificial sequence
  Synthetic sequence Probe
gtttaatctcgtcctcttc

SEQ ID NO: 107 - DNA, 19 bp, Artificial sequence
  Synthetic sequence Probe
acactctcagtaacttgta

SEQ ID NO: 108 - DNA, 19 bp, Artificial sequence
  Synthetic sequence Probe
acactctcaataacttgta

SEQ ID NO: 109 - DNA, 19 bp, Artificial sequence
  Synthetic sequence Probe
tacaagttattgagagtgt

SEQ ID NO: 110 - DNA, 19 bp, Artificial sequence
  Synthetic sequence Probe
tacaagttactgagagtgt

SEQ ID NO: 111 - DNA, 17 bp, Artificial sequence
  Synthetic sequence Probe
cactctcagtaacttgt

SEQ ID NO: 112 - DNA, 17 bp, Artificial sequence
  Synthetic sequence Probe
cactctcaataacttgt

SEQ ID NO: 113 - DNA, 19 bp, Artificial sequence
  Synthetic sequence Probe
tgggtgagtcacgacgcat

SEQ ID NO: 114 - DNA, 19 bp, Artificial sequence
  Synthetic sequence Probe
atgcgtcgtgactcaccca

SEQ ID NO: 115 - DNA, 19 bp, Artificial sequence
  Synthetic sequence Probe
tgggtgagttacgacgcat

SEQ ID NO: 116 - DNA, 19 bp, Artificial sequence
  Synthetic sequence Probe
atgcgtcgtaactcaccca

SEQ ID NO: 117 - DNA, 17 bp, Artificial sequence
  Synthetic sequence Probe
gggtgagtcacgacgca

SEQ ID NO: 118 - DNA, 17 bp, Artificial sequence
  Synthetic sequence Probe
gggtgagttacgacgca

SEQ ID NO: 119 - DNA, 19 bp, Artificial sequence
  Synthetic sequence Probe
tgagtcacgacgcatgaat

SEQ ID NO: 120 - DNA, 19 bp, Artificial sequence
  Synthetic sequence Probe
tgagttacgacgcatgaat

SEQ ID NO: 121 - DNA, 19 bp, Artificial sequence
  Synthetic sequence Probe
aatctgggtgagtcacgac

SEQ ID NO: 122 - DNA, 19 bp, Artificial sequence
  Synthetic sequence Probe
aatctgggtgagttacgac

SEQ ID NO: 123 - DNA, 19 bp, Artificial sequence
  Synthetic sequence Probe
ggtgagtcacgacgcatga

SEQ ID NO: 124 - DNA, 19 bp, Artificial sequence
  Synthetic sequence Probe
ggtgagttacgacgcatga

SEQ ID NO: 125 - DNA, 19 bp, Artificial sequence
  Synthetic sequence Probe
tctgggtgagtcacgacgc

SEQ ID NO: 126 - DNA, 19 bp, Artificial sequence
  Synthetic sequence Probe
tctgggtgagttacgacgc

SEQ ID NO: 127 - DNA, 19 bp, Artificial sequence
  Synthetic sequence Probe
catgttcactaaaacacca

SEQ ID NO: 128 - DNA, 19 bp, Artificial sequence
  Synthetic sequence Probe
tggtgttttagtgaacatg

SEQ ID NO: 129 - DNA, 19 bp, Artificial sequence
  Synthetic sequence Probe
catgttcaccaaaacacca

SEQ ID NO: 130 - DNA, 19 bp, Artificial sequence
  Synthetic sequence Probe
tggtgttttggtgaacatg

SEQ ID NO: 131 - DNA, 17 bp, Artificial sequence
  Synthetic sequence Probe
atgttcactaaaacacc

SEQ ID NO: 132 - DNA, 17 bp, Artificial sequence
  Synthetic sequence Probe
atgttcaccaaaacacc

SEQ ID NO: 133 - DNA, 19 bp, Artificial sequence
  Synthetic sequence Probe
ttcactaaaacaccatggc

SEQ ID NO: 134 - DNA, 19 bp, Artificial sequence
  Synthetic sequence Probe
ttcaccaaaacaccatggc

SEQ ID NO: 135 - DNA, 19 bp, Artificial sequence
  Synthetic sequence Probe
gagtacatgttcactaaaa

SEQ ID NO: 136 - DNA, 19 bp, Artificial sequence
  Synthetic sequence Probe
gagtacatgttcaccaaaa

SEQ ID NO: 137 - DNA, 19 bp, Artificial sequence
  Synthetic sequence Probe
tgttcactaaaacaccatg

SEQ ID NO: 138 - DNA, 19 bp, Artificial sequence
  Synthetic sequence Probe
tgttcaccaaaacaccatg

SEQ ID NO: 139 - DNA, 19 bp, Artificial sequence
  Synthetic sequence Probe
tacatgttcactaaaacac

SEQ ID NO: 140 - DNA, 19 bp, Artificial sequence
  Synthetic sequence Probe
tacatgttcaccaaaacac

SEQ ID NO: 141 - DNA, 19 bp, Artificial sequence
  Synthetic sequence Probe
ttcatgacccgttaactcc

SEQ ID NO: 142 - DNA, 19 bp, Artificial sequence
  Synthetic sequence Probe
ggagttaacgggtcatgaa

SEQ ID NO: 143 - DNA, 19 bp, Artificial sequence
  Synthetic sequence Probe
ttcatgacctgttaactcc

SEQ ID NO: 144 - DNA, 19 bp, Artificial sequence
  Synthetic sequence Probe
ggagttaacaggtcatgaa

SEQ ID NO: 145 - DNA, 17 bp, Artificial sequence
  Synthetic sequence Probe
tcatgacccgttaactc

SEQ ID NO: 146 - DNA, 17 bp, Artificial sequence
  Synthetic sequence Probe
tcatgacctgttaactc

SEQ ID NO: 147 - DNA, 19 bp, Artificial sequence
  Synthetic sequence Probe
atgacccgttaactcccct

SEQ ID NO: 148 - DNA, 19 bp, Artificial sequence
  Synthetic sequence Probe
atgacctgttaactcccct

SEQ ID NO: 149 - DNA, 19 bp, Artificial sequence
  Synthetic sequence Probe
ttattcatgacccgttaac

SEQ ID NO: 150 - DNA, 19 bp, Artificial sequence
  Synthetic sequence Probe
ttattcatgacctgttaac

SEQ ID NO: 151 - DNA, 19 bp, Artificial sequence
  Synthetic sequence Probe
catgacccgttaactcccc

SEQ ID NO: 152 - DNA, 19 bp, Artificial sequence
  Synthetic sequence Probe
catgacctgttaactcccc

SEQ ID NO: 153 - DNA, 19 bp, Artificial sequence
  Synthetic sequence Probe
tattcatgacccgttaact

SEQ ID NO: 154 - DNA, 19 bp, Artificial sequence
  Synthetic sequence Probe
tattcatgacctgttaact

SEQ ID NO: 155 - DNA, 19 bp, Artificial sequence
  Synthetic sequence Probe
gtacattgtattgtagatg

SEQ ID NO: 156 - DNA, 19 bp, Artificial sequence
  Synthetic sequence Probe
catctacaatacaatgtac

SEQ ID NO: 157 - DNA, 19 bp, Artificial sequence
  Synthetic sequence Probe
gtacattgtgttgtagatg

SEQ ID NO: 158 - DNA, 19 bp, Artificial sequence
  Synthetic sequence Probe
catctacaacacaatgtac

SEQ ID NO: 159 - DNA, 17 bp, Artificial sequence
  Synthetic sequence Probe
tacattgtattgtagat

SEQ ID NO: 160 - DNA, 17 bp, Artificial sequence
  Synthetic sequence Probe
tacattgtgttgtagat

SEQ ID NO: 161 - DNA, 19 bp, Artificial sequence
  Synthetic sequence Probe
attgtattgtagatgtttg

SEQ ID NO: 162 - DNA, 19 bp, Artificial sequence
  Synthetic sequence Probe
attgtgttgtagatgtttg

SEQ ID NO: 163 - DNA, 19 bp, Artificial sequence
  Synthetic sequence Probe
ggtaggtacattgtattgt

SEQ ID NO: 164 - DNA, 19 bp, Artificial sequence
  Synthetic sequence Probe
ggtaggtacattgtgttgt

SEQ ID NO: 165 - DNA, 19 bp, Artificial sequence
  Synthetic sequence Probe
acattgtattgtagatgtt

SEQ ID NO: 166 - DNA, 19 bp, Artificial sequence
  Synthetic sequence Probe
acattgtgttgtagatgtt

SEQ ID NO: 167 - DNA, 19 bp, Artificial sequence
  Synthetic sequence Probe
aggtacattgtattgtaga

SEQ ID NO: 168 - DNA, 19 bp, Artificial sequence
  Synthetic sequence Probe
aggtacattgtgttgtaga

SEQ ID NO: 169 - DNA, 19 bp, Artificial sequence
  Synthetic sequence Probe
cctgaccactggtctcttc

SEQ ID NO: 170 - DNA, 19 bp, Artificial sequence
  Synthetic sequence Probe
gaagagaccagtggtcagg

SEQ ID NO: 171 - DNA, 19 bp, Artificial sequence
  Synthetic sequence Probe
cctgaccaccggtctcttc

SEQ ID NO: 172 - DNA, 19 bp, Artificial sequence
  Synthetic sequence Probe
gaagagaccggtggtcagg

SEQ ID NO: 173 - DNA, 21 bp, Artificial sequence
  Synthetic sequence Probe
tcctgaccactggtctcttca

SEQ ID NO: 174 - DNA, 21 bp, Artificial sequence
  Synthetic sequence Probe
tcctgaccaccggtctcttca

SEQ ID NO: 175 - DNA, 19 bp, Artificial sequence
  Synthetic sequence Probe
tgtcctgaccactggtctc

SEQ ID NO: 176 - DNA, 19 bp, Artificial sequence
  Synthetic sequence Probe
ggggtgtcctgaccactgg

SEQ ID NO: 177 - DNA, 19 bp, Artificial sequence
  Synthetic sequence Probe
tgtcctgaccaccggtctc

SEQ ID NO: 178 - DNA, 19 bp, Artificial sequence
  Synthetic sequence Probe
ggggtgtcctgaccaccgg

SEQ ID NO: 179 - DNA, 19 bp, Artificial sequence
  Synthetic sequence Probe
gaccactggtctcttcaca

SEQ ID NO: 180 - DNA, 19 bp, Artificial sequence
  Synthetic sequence Probe
gaccaccggtctcttcaca

SEQ ID NO: 181 - DNA, 19 bp, Artificial sequence
  Synthetic sequence Probe
ccactggtctcttcacagg

SEQ ID NO: 182 - DNA, 19 bp, Artificial sequence
  Synthetic sequence Probe
ccaccggtctcttcacagg

SEQ ID NO: 183 - DNA, 45 bp, Artificial sequence
  Synthetic sequence Primer including tag
aaccttcaactacacggctcacctgcccttgtaagttgggtggaa

SEQ ID NO: 184 - DNA, 43 bp, Artificial sequence
  Synthetic sequence Primer including tag
aaggagattatgtaccgaggaagaagtcttcaggtgggggaca

SEQ ID NO: 185 - DNA, 45 bp, Artificial sequence
  Synthetic sequence Primer including tag
aaccttcaactacacggctcacctggactgatctgtgccttctgc

SEQ ID NO: 186 - DNA, 44 bp, Artificial sequence
  Synthetic sequence Primer including tag
aaggagattatgtaccgaggaagaagtccccggaataacgaaag

SEQ ID NO: 187 - DNA, 45 bp, Artificial sequence
  Synthetic sequence Primer including tag
aaccttcaactacacggctcacctgggacactactgttagagcca

SEQ ID NO: 188 - DNA, 45 bp, Artificial sequence
  Synthetic sequence Primer including tag
aaggagattatgtaccgaggaagaaagttgtcgccatctttgagg

SEQ ID NO: 189 - DNA, 46 bp, Artificial sequence
  Synthetic sequence Primer including tag
aaccttcaactacacggctcacctgtggatagttgtgaggctttcc

SEQ ID NO: 190 - DNA, 47 bp, Artificial sequence
  Synthetic sequence Primer including tag
aaggagattatgtaccgaggaagaacatgaaccttccagaagagatg

SEQ ID NO: 191 - DNA, 45 bp, Artificial sequence
  Synthetic sequence Primer including tag
aaccttcaactacacggctcacctgtcaattgcctatgccttgtg

SEQ ID NO: 192 - DNA, 45 bp, Artificial sequence
  Synthetic sequence Primer including tag
aaggagattatgtaccgaggaagaacggaggtgaagaacacaaca

SEQ ID NO: 193 - DNA, 45 bp, Artificial sequence
  Synthetic sequence Primer including tag
aaccttcaactacacggctcacctgtccagtttttggttttcagc

SEQ ID NO: 194 - DNA, 45 bp, Artificial sequence
  Synthetic sequence Primer including tag
aaggagattatgtaccgaggaagaactgagcacctctgtggatca

SEQ ID NO: 195 - DNA, 49 bp, Artificial sequence
  Synthetic sequence Primer including tag
aaggagattatgtaccgaggaagaacaaatatgtctttagcagataagc

SEQ ID NO: 196 - DNA, 43 bp, Artificial sequence
  Synthetic sequence Primer including tag
aaccttcaactacacggctcacctgggcctgtggagctgactg

SEQ ID NO: 197 - DNA, 43 bp, Artificial sequence
  Synthetic sequence Primer including tag
aaggagattatgtaccgaggaagaaacggccaatcaacgtcat

SEQ ID NO: 198 - DNA, 45 bp, Artificial sequence
  Synthetic sequence Primer including tag
aaccttcaactacacggctcacctgcagtttgttggtgcaagctc

SEQ ID NO: 199 - DNA, 45 bp, Artificial sequence
  Synthetic sequence Primer including tag
aaggagattatgtaccgaggaagaactcaggtgagggggatctct